(19) United States
(12) Patent Application Publication
(10) Pub. No.: US 2022/0356256 A1
(43) Pub. Date: Nov. 10, 2022

(54) Frizzled receptor antibodies and uses thereof

(71) Applicant: MODMAB THERAPEUTICS INC., Toronto (CA)

(72) Inventors: Sachdev S. SIDHU, Toronto (CA); Guohua PAN, Oakville (CA); Jason MOFFAT, Toronto (CA); Amandeep GAKHAL, San Jose, CA (US); Stephane ANGERS, Mississauga (CA); Zachary STEINHART, Thornhill (CA); Zvezdan PAVLOVIC, Guelph (CA); Jarrett ADAMS, Toronto (CA)

(21) Appl. No.: 17/634,891

(22) PCT Filed: Aug. 12, 2020
(86) PCT No.: PCT/CA2020/051103

§ 371 Date: Feb. 11, 2022

Related U.S. Application Data
(60) Provisional application No. 62/886,292, filed on Aug. 13, 2019.
(60) Provisional application No. 62/885,781, filed on Aug. 12, 2019.

(51) Int. Cl. C07K 16/28; A61K 47/68; G01N 33/574; A61P 35/00

(52) U.S. Cl. CPC C07K 16/2863; A61K 47/6849; G01N 33/57407; A61P 35/00; C07K 2317/92; C07K 2317/24; C07K 2317/622; C07K 2317/35; C07K 2317/31; C07K 2317/41; C07K 2317/76; C07K 2317/55

(57) ABSTRACT

Isolated antibodies and immunoconjugates that specifically bind Frizzled receptor (FZD) 4 cysteine rich domain (CRD) comprising a light chain variable region and a heavy chain variable region, the heavy chain variable region comprising complementarity determining regions CDR-H1, CDR-H2 and CDR-H3, the light chain variable region comprising complementarity determining region CDR-L1, CDR-L2 and CDR-L3, and with the amino acid sequences of said CDRs comprising or consisting of sequences selected from sequences in Table 1a or 3a. Methods of using the antibodies and immunoconjugates are also provided.

### REFERENCE TO RELATED APPLICATIONS

This application claims the benefit of the priority dates of U.S. provisional application 62/885,781, filed Aug. 12, 2019, and U.S. provisional application 62/886,292, filed Aug. 13, 2019, the contents of which are incorporated herein by reference in their entirety.

### FIELD

The disclosure pertains to antibodies that bind a frizzled receptor and particularly to antibodies that bind to the frizzled receptor 4 Cysteine Rich Domain and uses thereof.

### BACKGROUND

The frizzled receptors (FZDs), an important class of seven transmembrane receptors that are involved in many important biological processes such as development, cell proliferation, survival, migration and stem cell maintenance. The signaling pathways are activated when Wnt ligands interact with the Frizzled family of seven transmembrane receptors and control stem and progenitor cell renewal and cell differentiation during embryonic development and tissue homeostasis in adult animals. Abnormal expression and signaling of these receptors and their ligands (Wnts) have been associated with numerous cancers, including colon, lung, breast and ovarian cancers. Frequently, multiple Wnt ligands and/or frizzled receptors are up-regulated and lead to aberrant signaling that drives tumorigenesis. Therefore, inhibition of multiple frizzled receptors may be necessary to achieve better anti-cancer efficacy. In addition, frizzled receptors have also been implicated in cancer stem cells, a small population of cancer cells that are thought to be responsible for drug resistance, tumor relapse and metastasis. Thus, inhibition of FZD receptors (either one or multiple receptors) including FZD4 may be an effective way to target cancer stem cells and to treat various types of cancer.

Wnt signaling leads to the activation of the canonical and non-canonical signaling pathways. The non-canonical pathway activates signaling molecules that do not involve the nucleus or transcription but rather activate cytoplasmic signals that regulate the cytoskeleton and calcium levels. This pathway primarily plays a role in regulating cell polarity or migration.

The canonical pathway predominantly controls transcriptional activity by regulating the cytoplasmic levels of β-catenin. In unstimulated conditions, β-catenin is associated with a destruction complex, comprised of Axin, APC, CD1 and GSKβ, which results in the phosphorylation, ubiquitylation and proteasomal degradation of β-catenin. Wnt signaling is active when Wnt binds to frizzled (FDZ), a 7-pass transmembrane receptor, and to a co-receptor low density lipoprotein receptor-related protein (either LRP5 or LRP6). This signaling destabilizes the complex, in part by attracting disheveled (Dsh/Dvl) to the plasma membrane, resulting in the accumulation of β-catenin, which then travels to the nucleus and activates TCF/LEF-mediated transcription.

Several cancers in humans are caused by mutations within cytoplasmic components of the WNT pathway that result in ligand-independent activation of Wnt target genes. For example, inactivating APC mutations and activating β-catenin mutations are the major underlying cause of colorectal cancers in humans. Since the pathway is activated downstream of the cell surface receptors, developing targeted therapies against the Wnt pathway has proven challenging. Recently however, cancer causing mutations in negative regulators of Wnt signaling, RNF43 (colon, endometrial, pancreas, stomach, ovary, liver cancers and its homolog ZNRF3 (adrenocortical carcinoma and osteosarcoma), have been identified and implicate ligand-dependent tumor growth. Indeed, RNF43 and ZNRF3 are Wnt target genes coding for transmembrane E3 ubiquitin ligases targeting Frizzled receptors, whose loss-of-function mutations lead to high expression of FZDs and may sensitize tumor cells to the inhibition of Wnt-dependent signaling.

### SUMMARY

The following summary is intended to introduce the reader to various aspects of the disclosure, but not to define or delimit any invention.

In one aspect, the disclosure provides an antibody that specifically binds a cysteine rich domain (CRD) of each of one or a plurality of human Frizzled receptors selected from FZD 1, 2, 4, 5, 7, 8 and 9, comprising a light chain variable region and/or a heavy chain variable region, the heavy chain variable region comprising complementarity determining regions CDR-H1, CDR-H2 and CDR-H3, the light chain variable region comprising complementarity determining region CDR-L1, CDR-L2 and CDR-L3, and with the amino acid sequences of said CDRs comprising or consisting of sequences selected from sequences in Table 1a or 3a. In one embodiment the CDRs comprise (a) a full sequence set or (b) a light chain sequence set or (c) heavy chain sequence set selected from the antibodies identified in Table 1a or 3a.

In another embodiment, the CDRs comprise or consist of the sequences, wherein:

CDR-H1 is selected from the group consisting of ISYYYM, IYSYYM, LSYYYM, IYYYSI, LYSYYM, LSSYSM, ISYYYI, LSYSSM, IYYYYM, LYYYSI, ISSYYI, FSSSSI, LSYYSI, LYSYYI, LSSYYM, LSYYYI, ISSYYM, LSYYSM, LYSYSI, LYYYYI, IYSYYI, ISYYYI and ISYYSM;

CDR-H2 is selected from the group consisting of SIYSYYGYTY, SIYSSSSSTY, SIYPSSSYTY, SIYSSSSYTS, YISSYSGSTY, SIYSSYGYTY, YISSYYGYTY, SIYPSSSSTY, SIYSSSGYTY, YISSYSGSTS, SISSYYGSTY, SIYSYYGSTY, SIYPYSGYTY, YISPYYGYTS, SISSSSGYTY, SIYSYSSSTY, SISPSSSYTY, YISPYYGYTY, SISPYSSSTY, SIYSSYGSTY, SIYSSSSYTY, SIYPSSGYTY, SIYPYSGSTY, SIYPSYGSTY, YISSYSSYTY, SIYSYYSSTY, YISSSYGYTS, SISPYSSYTY, YISPYSGYTS, SIYPYYSYTY, SISPYYGYTS, SISPSYSSTY, SISSSYSSTY, SIYPYSGSTS, SISSYYSSTS and SIYSYSGYTY;

CDR-H3 is selected from the group consisting of SSFSWAM, SSFYWAL, SWFGWGI, YWFSYGYASYPAF, HPWYGM, SAFYWAL, PAPGHWGF, SSFFWAM, SAFYWAM, HFFAM, SWWAWAF, SAFGWAL, SSFFFAM, PYYWSGGF, HPSSSWFSFGAL, SAFYWAF, SSYAWAM, SSFYWAI, SPWGSGWAGF, PAVWVGL, SWVFWAL, SWVYWGM, SWVYWAL, SSYAWAI, SSFYWAM, HGASFGSGAPAF, SCFFWAM, WAFFGL, SSFYFAM, SAFSWAI, SGFYWAL, PSVGYAAF, SWVGWGL, SSVGYVAM, SWVYWAF, YYYSSSVYFWYAAL, SSFFWAI, SWVYWAI, SWVGWGI, SSVYWAL, WGGWGSGGYFYAAL, FWYPGM and SSFAWAF;

CDR-L1 is SVSSA;

CDR-L2 is SASSLYS; and

CDR-L3 is selected from the group consisting of HPWSGGYLI, PVGYWGVPI, VSGGAHALI, VSSAYPI, FWGVPI, SYYHYAALI, WYYAPI, SHSYSLI, SGYGPF, SWSSPI, HYSVYASLI, PHPPSLI, VAYSHVGLI, GYGAPI, SWYSLI, PGYLF, VWFGLI, VYYGSPLF, HAHSPLI, SSAYYPF, GHASPI, SSGGWSLI, VAWSSFLI, SVAAASLI, SGWWGVSLI, SYAAYLF, HGSLF, YAGVSNLF, GWPYSALF, SGYYPSLF, SYHSGSGLI, HGYSASLI, APGWALF, GHSSPI, GWPSLF, VPGYPVPI, HYYSHLI, GPASSLI, SVGSSYYLI, YYGPWVLI, AASWGYPF, HWSYPI and GGWGPF.

In yet another embodiment, the CDRs comprise or consist of the sequences, wherein:

CDR-H1 is selected from the group consisting of ISYYYM, IYSYYM, LSYYYM, IYYYSI, LYSYYM, LSSYSM, ISYYYI, LSYSSM, IYYYYM, LYYYSI, ISSYYI, FSSSSI, LSYYSI, LYSYYI, LSSYYM, LSYYYI, ISSYYM, LSYYSM, LYSYSI, LYYYYI, IYSYYI, ISYSYI and ISYYSM;

CDR-H2 is selected from the group consisting of SIYSYYGYTY, SIYSSSSSTY, SIYPSSSYTY, SIYSSSSYTS, YISSYSGSTY, SIYSSYGYTY, YISSYYGYTY, SIYPSSSSTY, SIYSSSGYTY, YISSYSGSTS, SISSYYGSTY, SIYSYYGSTY, SIYPYSGYTY, YISPYYGYTS, SISSSSGYTY, SIYSYSSSTY, SISPSSSYTY, YISPYYGYTY, SISPYSSSTY, SIYSSYGSTY, SIYSSSSYTY, SIYPSSGYTY, SIYPYSGSTY, SIYPSYGSTY, YISSYSSYTY, SIYSYYSSTY, YISSSYGYTS, SISPYSSYTY, YISPYSGYTS, SIYPYYSYTY, SISPYYGYTS, SISPSYSSTY, SISSSYSSTY, SIYPYSGSTS, SISSYYSSTS and SIYSYSGYTY;

CDR-H3 is selected from the group consisting of SSFSWAM, SSFYWAL, SWFGWGI, YWFSYGYASYPAF, HPWYGM, SAFYWAL, PAPGHWGF, SSFFWAM, SAFYWAM, HFFAM, SWWAWAF, SAFGWAL, SSFFFAM, PYYWSGGF, HPSSSWFSFGAL, SAFYWAF, SSYAWAM, SSFYWAI, SPWGSGWAGF, PAVWVGL, SWVFWAL, SWVYWGM, SWVYWAL, SSYAWAI, SSFYWAM, HGASFGSGAPAF, SCFFWAM, WAFFGL, SSFYFAM, SAFSWAI, SGFYWAL, PSVGYAAF, SWVGWGL, SSVGYVAM, SWVYWAF, YYYSSSVYFWYAAL, SSFFWAI, SWVYWAI, SWVGWGI, SSVYWAL, WGGWGSGGYFYAAL, FWYPGM and SSFAWAF;

CDR-L1 is SVSSA;

CDR-L2 is SASSLYS; and

CDR-L3 is selected from the group consisting of HPWSGGYLI, PVGYWGVPI, VSGGAHALI, VSSAYPI, FWGVPI, SYYHYAALI, WYYAPI, SHSYSLI, SGYGPF, SWSSPI, HYSVYASLI, PHPPSLI, VAYSHVGLI, GYGAPI, SWYSLI, PGYLF, VWFGLI, VYYGSPLF, HAHSPLI, SSAYYPF, GHASPI, SSGGWSLI, VAWSSFLI, SVAAASLI, SGWWGVSLI, SYAAYLF, HGSLF, YAGVSNLF, GWPYSALF, SGYYPSLF, SYHSGSGLI, HGYSASLI, APGWALF, GHSSPI, GWPSLF, VPGYPVPI, HYYSHLI, GPASSLI, SVGSSYYLI, YYGPWVLI, AASWGYPF, HWSYPI and GGWGPF.

In a further embodiment, the disclosure provides an antibody as previously described comprising a heavy chain variable region comprising:
i) a heavy chain amino acid sequence as set forth in Table 2;
ii) an amino acid sequence with at least 50%, at least 60%, at least 70%, at least 80%, or at least 90% sequence identity to the heavy chain amino acid sequence as set forth in Table 2, wherein the CDR sequences are a CDR sequence set as set forth in Table 1a or 3a, or
iii) a conservatively substituted amino acid sequence of i) wherein the CDR sequences are a CDR sequence set as set forth in Table 1a or 3a.

In a further embodiment, the disclosure provides an antibody further comprising a light chain variable region comprising:
i) a light chain amino acid sequence as set forth in Table 2,
ii) an amino acid sequence with at least 50%, at least 60%, at least 70%, at least 80%, or at least 90% sequence identity to the light chain amino acid sequence as set forth in Table 2, wherein the CDR sequences are a CDR sequence set as set forth in Table 1a or 3a, or
iii) a conservatively substituted amino acid sequence of i) wherein the CDR sequences are a CDR sequence set as set forth in Table 1a or 3a.

In a further embodiment, the disclosure provides an antibody that specifically binds FZD4. In a further embodiment the antibody that specifically binds FZD4 the CDR sequences are a CDR sequence set of an antibody selected from antibodies 5017, 5027, 5030, 6499, 5038, 5040-5044, 5046, 5047, 5049-5053, 5055, 5058-5064, 5066, 5068-5072, 5077-5080 or 5081.

In a further embodiment, the disclosure provides an antibody that specifically binds FZD4 and at least one other receptor selected from FZD1, 2, 5, 7, 8 and 9. In yet a further embodiment, the CDR sequences are a CDR sequence set of an antibody selected from antibodies 5014, 5016, 5018-5023, 5025, 5028, 5029, 5031, 5034, 5035, 5036, 5037, 6494, 6495, 6496, 6497, 6498, 6500, 5039, 5045, 5048, 5054, 5056, 5057, 5067, and 5073-5076. In yet a further embodiment, the antibody preferentially binds Frizzled 4 (FZD4) relative to another FZD receptor. In yet a further embodiment, the antibody comprises the CDR sequences are a CDR sequence set of an antibody selected from antibodies 5028, 5029, 5031, 5034, 5035, 6497, 6498, 5039, 5045, 5048, 5054, 5056, 5057, 5067, 5073, 5074, 5075. In yet a further embodiment, the antibody has a binding affinity measured by surface plasmon resonance of between about 0.2 nM and about 15.3 nM.

In yet a further embodiment, the CDR sequences are a CDR sequence set of an antibody selected from antibodies 5017, 5027, 5030, 6499, 5038, 5040-5044, 5046, 5047, 5049-5053, 5055, 5058-5064, 5066, 5068-5072, 5077-5080 or 5081.

In yet a further embodiment, the antibody is monoclonal, humanized, single chain, antibody fragment, polyvalent, bispecific, comprises a non-natural glycosylation pattern, comprises a cysteine substitution or addition or blocks the binding of Wnt to FZD. In yet a further embodiment, the antibody fragment is selected from the group consisting of fragment selected from Fab, Fab′, F(ab′)2, scFv, dsFv, ds-scFv, dimers, nanobodies, minibodies, diabodies, and multimers thereof. In yet a further embodiment, the polyvalent antibody is divalent, trivalent or tetravalent. In yet a further embodiment, the cysteine substitution is present is present in the constant region or the framework region. In yet a further embodiment, the bispecific antibody further binds LRP 5 and/or 6. In yet a further embodiment, the antibody comprises a non-natural glycosylation pattern. In yet a further embodiment, the cysteine substitution is in the constant or framework region. In yet a further embodiment the antibody as described herein blocks the binding of Wnt to FZD.

In another aspect, the disclosure provides an immunoconjugate comprising an antibody as described herein and a detectable label or cytotoxic agent. In one embodiment, the cytotoxic agent is selected from selected from maytansinoid, auristatin, dolastatin, tubulysin, cryptophycin, pyrrolobenzodiazepine (PBD) dimer, indolinobenzodiazepine dimer, alpha-amanitin, trichothene, SN-38, duocarmycin, CC1065, calicheamincin, an enediyne antibioatic, taxane, doxorubicin derivatives, anthracycline and stereoisomers, azanofide, isosteres, analogs or derivatives thereof.

In another aspect, the disclosure provides a nucleic acid encoding an antibody as described herein. In one embodiment, one or more of the CDR sequences encoded by the nucleic acid is/are described in Table 1b, 1c, 3b and 3c.

In another embodiment, the antibody encoded by the nucleic acid comprises a light chain variable region encoded by nucleic acid comprising:
i) a heavy chain nucleic acid sequence as set forth in Table 2,
ii) a nucleic acid sequence with at least 50%, at least 60%, at least 70%, at least 80%, or at least 90% sequence identity to the light chain nucleic acid sequence as set forth in Table 2, wherein the CDR sequences are a CDR sequence set as set forth in n Table 1a or 3a, or
iii) a codon-degenerate nucleic acid sequence of i) wherein the CDR sequences are a CDR sequence set as set forth in Table 1a or 3a.

In another embodiment, the antibody encoded by the nucleic acid comprises a light chain variable region encoded by nucleic acid comprising:
i) a light chain nucleic acid sequence as set forth in Table 2,
ii) a nucleic acid sequence with at least 50%, at least 60%, at least 70%, at least 80%, or at least 90% sequence identity to the light chain nucleic acid sequence as set forth in Table 2, wherein the CDR sequences are a CDR sequence set as set forth in n Table 1a or 3a, or
iii) a codon-degenerate nucleic acid sequence of i) wherein the CDR sequences are a CDR sequence set as set forth in Table 1a or 3a.

In yet another aspect, the disclosure provides a vector comprising an expression control sequence operative linked to the nucleic acid encoding an antibody described herein.

In yet a further aspect, the disclosure provides a host cell comprising recombinant nucleic acid molecule comprising an expression control sequence operatively linked to the nucleic acid encoding an antibody described herein. In one embodiment, the host cell is a Chinese Hamster Ovary (CHO) cell.

In yet a further aspect, the disclosure provides a method for making an anti-FZD antibody comprising culturing a host cell as described herein.

In yet a further aspect, the disclosure provides a composition comprising one or more antibodies, immunoconjugates, nucleic acids, vectors or host cells described herein optionally with a suitable diluent. In one embodiment, the composition comprises one or more antibodies or immunoconjugates, optionally wherein the composition is a pharmaceutical composition.

In yet a further aspect, the disclosure provides a kit comprising one or more antibodies, immunoconjugates, nucleic acids, vectors or host cells described herein.

In yet a further aspect, the disclosure provides a method of detecting FZD expression, the method comprising contacting a sample comprising one or more cells with one or more antibody or immunoconjugate described herein under conditions permissive for forming an antibody:cell complex and detecting the presence of any antibody complex. In one embodiment, the detection method is by immunofluorescence. In another embodiment, the detection method is by flow cytometry. In yet another embodiment, the method is for detecting FZD4 expression and the antibody or immunoconjugate comprises a CDR sequence set corresponding to an antibody selected from 5017, 5027, 5030, 6499, 5038, 5040-5044, 5046, 5047, 5049-5053, 5055, 5058-5064, 5066, 5068-5072, and 5077-5081.

In yet a further aspect, the disclosure provides a method of inhibiting Wnt ligand binding to a FZD receptor, disrupting a Wnt signalling pathway, inhibiting Wnt-induced transcriptional activity, inhibiting activation of disheveled, promoting preservation of the beta-catenin destruction complex of the beta-catenin destruction complex, promoting accumulation of beta-catenin or inhibiting growth of a cell, the method comprising contacting a cell expressing a FZD receptor with an antibody or immunoconjugate described herein. In another aspect, the disclosure provides an antibody or immunoconjugate described herein for use in inhibiting Wnt ligand binding to a FZD receptor, disrupting a Wnt signalling pathway, inhibiting Wnt-induced transcriptional activity, inhibiting activation of disheveled, promoting preservation of the beta-catenin destruction complex of the beta-catenin destruction complex, promoting accumulation of beta-catenin or inhibiting growth of a cell. In a further aspect, the present disclosure provides a use of an antibody or immunoconjugate described herein for inhibiting Wnt ligand binding to a FZD receptor, disrupting a Wnt signalling pathway, inhibiting Wnt-induced transcriptional activity, inhibiting activation of disheveled, promoting preservation of the beta-catenin destruction complex of the beta-catenin destruction complex, promoting accumulation of beta-catenin or inhibiting growth of a cell. In yet a further aspect, the present disclosure provides a use of an antibody or immunoconjugate described herein in the manufacture of a medicament for inhibiting Wnt ligand binding to a FZD receptor, disrupting a Wnt signalling pathway, inhibiting Wnt-induced transcriptional activity, inhibiting activation of disheveled, promoting preservation of the beta-catenin destruction complex of the beta-catenin destruction complex, promoting accumulation of beta-catenin or inhibiting growth of a cell. In one embodiment, the Wnt ligand is Wnt3a. In another embodiment, the antibody or immunoconjugate comprises a CDR sequence set corresponding to an antibody selected from a) 5017, 5027, 5030, 6499, 5038, 5040-5044, 5046, 5047, 5049-5053, 5055, 5058-5064, 5066, 5068-5072, and 5077-5081 or b) 5014, 5016, 5018-5023, 5025, 5028, 5029, 5031, 5034, 5035, 5036, 5037, 6494, 6495, 6496, 6497, 6498, 6500, 5039, 5045, 5048, 5054, 5056, 5057, 5067, and 5073-5076.

In yet a further aspect, the disclosure provides method of treating cancer in a subject in need thereof comprising administering to the subject an effective amount of a pharmaceutical composition comprising an antibody or immunoconjugate as described herein. In another aspect, the disclosure provides a use of an antibody or immunoconjugate as described herein for treating cancer. In yet another aspect, the present disclosure provides an antibody or immunoconjugate as described herein for use in treating cancer. In yet another aspect, the disclosure provides a use of an antibody or immunoconjugate as described herein in the manufacture of a medicament for treating cancer. In one embodiment, the cancer is selected from acute myeloid leukemia, neuroblastoma, liver cancer, lung cancer, endometrial cancer, salivary adenoid cystic carcinoma cancer, colorectal cancer, prostate cancer, glioblastoma, bladder cancer cervical cancer, pancreatic cancer, colon cancer, breast cancer, esophageal cancer, glioma, gastric cancer, astrocytoma, and osteosarcoma. In another embodiment, the method or use comprises an antibody or immunoconjugate that specifically binds FZDs 1, 2, 4, 5, 7, 8 and 9 in at least one assay, and inhibits Wnt3a-induced signalling in at least one assay, optionally wherein the antibody or immunoconjugate is the antibody or immunoconjugate is described herein. In yet another embodiment, antibody or immunoconjugate of the method or use comprises a CDR sequence set corresponding to an antibody selected from a) 5017, 5027, 5030, 6499, 5038, 5040-5044, 5046, 5047, 5049-5053, 5055, 5058-5064, 5066, 5068-5072, and 5077-5081 orb) 5014, 5016, 5018-5023, 5025, 5028, 5029, 5031, 5034, 5035, 5036, 5037, 6494, 6495, 6496, 6497, 6498, 6500, 5039, 5045, 5048, 5054, 5056, 5057, 5067, and 5073-5076. In yet a further embodiment, the antibody or immunoconjugate comprises a CDR sequence set corresponding to an antibody selected from 5019 and 5020. In yet a further embodiment, the cancer treated by the method or use comprises one or more cancer cells comprise a mutation in RNF43 gene and the antibody and the antibody or immunoconjugate comprises a CDR sequence set corresponding to antibody 5020.

Other features and advantages of the present disclosure will become apparent from the following detailed description. It should be understood, however, that the detailed description and the specific examples while indicating embodiments are given by way of illustration only, the scope of the claims should not be limited by the embodiments set forth in the examples, but should be given the broadest interpretation consistent with the description as a whole.

### BRIEF DESCRIPTION OF THE DRAWINGS

An embodiment of the present disclosure will now be described in relation to the drawings in which:

FIG. 1 is a graph showing the binding of phage clones to FZD4-CRD-Fc and Fc. Single colonies were used to inoculate 96-well culture plate, and overnight phage supernatants were diluted 1:2 in 0.05% Tween20/0.5% BSA/PBS (dilution buffer) to test for binding in an ELISA. Phage were detected with an anti-M13-HRP secondary antibody (1:5000 in dilution buffer) and plates were developed with TMB substrate and an acid stop. The absorbance at 450 nm was read for FZD4-Fc coated wells and control Fc coated wells, as indicated.

FIG. 2 is a graph showing the competitive ELISA binding of Anti-FZD4 Fabs. ELISAs were performed to estimate the affinity of the FZD4 Fab panel. 384-well ELISA plates were coated with FZD4 CRD-Fc (R&D systems) at 2 μg/ml in PBS overnight at 4 degrees C. Plates were blocked for one hour at room temperature with 0.5% BSA/PBS and then washed three times with 0.05% Tween20/PBS. Fabs at a final concentration of 0.5 μg/ml were pre-incubated for one hour at room temperature in a non-binding 96-well ELISA plate with indicated concentrations of FZD4 CRD-Fc in solution (in 0.05% Tween20/0.5% BSA/PBS) in solution. This Fab antigen mixture was transferred into the 384-well plates and incubated for 20 minutes at room temperature. Plates were washed six times and binding Fab was detected with an anti-FLAG-HRP secondary antibody (Sigma) at 1:5000 in 0.05% Tween20/0.5% BSA/PBS. Secondary antibody was incubated for 45 minutes at room temperature, plates were washed, and then developed with TMB substrate with an acid stop. The absorbance at 450 nm was read. The % binding was calculated as the absorbance of the Fab wells with competing soluble FZD4 CRD-Fc over the absorbance of Fab wells in the absence of competing soluble FZD4 CRD-Fc, multiplied by 100.

FIG. 3 is a series of immunofluorescent staining pictures showing the binding of Anti-FZD4 Fabs to the FZDs expressed on CHO cells. Fabs from FZD4 selections were tested for reactivity by immunofluorescent staining (IF) of a CHO over-expression line stably expressing the FZD4 CRD region as a GPI linked domain with a myc tag. Fabs were detected with an anti-F(ab′)₂-FITC secondary antibody (Jackson Immuno) and FITC staining is indicated by the white areas.

FIG. 4 is a series of immunofluorescent staining pictures showing the binding of Anti-FZD4 Fabs to the CHO cells. Fabs from FZD4 selections were tested for reactivity by immunofluorescent staining (IF) of a control CHO cell line stably transfected with the GPI linker and myc tag. Fabs were detected with an anti-F(ab′)₂-FITC secondary antibody (Jackson Immuno) and FITC staining is indicated by the white areas.

FIG. 5 is a table showing the binding of Fabs to FZDs determined by IF staining.

FIG. 6 is a series of immunofluorescent staining pictures showing the binding of Fabs 5019 and 5020 binding to FZDs in multiple human pancreatic cancer cell lines.

FIG. 7 is a series of graphs showing the binding of Fabs 5019 and 5020 to pancreatic cell lines by Flow Cytometry. Numbers indicate fold increase in MFI over the secondary Ab control.

FIG. 8A-D is a series of graphs showing the binding of anti-FZD4 Fabs to FZDs.

FIG. 9A-B is a table showing the binding of anti-FZD4 Fabs to FZDs determined by IF. CHO myc GPI cell lines. 200 nM Fabs. −=No binding; +=Really weak binder; ++=Weak binder; +++=Good Binder; ++++=Very Good Binder.

FIG. 10 is a table showing the binding affinity of anti-FZD4 Fabs to FZD4 determined by SPR.

FIG. 11 is a graph showing the binding of anti-FZD4 Fabs to pancreatic cancer cells determined by flow cytometry.

FIG. 12A-B is a series of graphs showing the inhibition of WNT5A binding by anti-FZD4 Fabs. (A) Wnt5a (R&D systems) was biotinylated using a commercial kit (Thermo 21329 EZ-link NHS-PEG4-Biotin) and excess biotin was removed by buffer exchange using a 3000MWCO Amicon filter. FZD4-CRD-Fc or control Fc protein (R&D systems) was diluted in 1% BSA/0.05% Tween20/PBS (dilution buffer) to the indicated concentrations and incubated with a constant amount of biotinylated Wnt5a for 1 hour at room temperature in a BSA blocked 96-well TC treated plate. Control wells in which buffer alone was added in lieu of biotinylated wnt5a were also included. Biotinylated Wnt5a was added at a final concentration of 150 ng/ul. Samples were transferred to pre-blocked streptavidin coated plates (R&D systems) and allowed to capture for 1 hour at room temperature. Wells were washed four times with 0.05% Tween20/PBS and then anti-Fc-HRP (1:5000 in dilution buffer, Jackson Immuno) was added to the wells for 45 min at room temperature. Wells were washed four times and developed with TMB reagent with an acid stop. The absorbance at 450 nm was read. (B) FZD4-CRD-Fc was diluted to a concentration previously determined in (A) to give an ELISA signal within a linear range and incubated with desired Fab or buffer controls for 1 hour at room temperature in 96-well TC plates pre-blocked with 1% BSA. As above, control wells with Fc protein were also included. Biotinylated wnt5a was added to the wells and plates were incubated for an additional hour. Control wells in which buffer alone was added in lieu of biotinylated wnt5a were also included. Biotinylated Wnt5a was added at a final concentration of 150 ng/ul. Fab proteins were at a final concentration of 400 nM with the following exceptions: Fab 6494 at 180 nM, Fab 6406 at 135 nM, and Fab 6500 at 159 nM. A negative control Fab specific for a different protein antigen was included, as well as a control for effects from the neutralized elution buffer (Fab buffer control) that Fab proteins were stored in. The percent binding was calculated.

FIG. 13 is a table showing the Effect of Fabs on beta-catenin driven transcription (TOPFLASH assay).

FIG. 14A-H is a series of graphs showing the effect of anti-FZD4 Fabs on proliferation of cancer cells.

FIG. 15 is a table showing the effect of anti-FZD4 Fabs on proliferation of pancreatic cancer cells as determined by SRB Sulforhodamine B) assay where “na” means not tested.

FIG. 16 is a summary table of anti-FZD4 Fabs where na means not tested. Anti-proliferative activity appears to be associated with binding to FZDs 1,2,4,5,7,8,9 and Inhibition of wnt3a activity. The most potent inhibitors as shown in this assay are 5014, 5019-5023 and 6495.

FIG. 17 is a graph showing that anti-FZD4 antibodies inhibit expression of Axin2, a Wnt pathway regulated gene in human pancreatic adenocarcinoma cell line (HPAF II). Gene expression (RT-qPCR) upon treatment with 200 nM Fab/IgG was normalized to beta actin.

FIG. 18A-B is a series of graphs showing IgG 5019 and 5020 proliferation inhibition. A) shows that IgG 5019 and 5020 inhibit proliferation of pancreatic cancer cells using alamar blue proliferation assays, 200 nM Fab/IgG and B) shows that IgG 5020 inhibits cell proliferation in a dose dependent manner.

FIG. 19 is a series of colony photos showing IgG 5020 inhibits colony formation.

FIG. 20 is a series of immunofluorescence staining pictures showing the binding of Fabs 5019 and 5020 to FZDs in PDAC patient derived xenografts (PDX) cell lines (GP2A and GP14A).

FIG. 21 is a graph showing the effect of Fab 5019, 5020 and IgG 5020 on proliferation of PDAC PDX cell lines in alamar blue proliferation assays, 200 nM Fab/IgG.

FIG. 22 shows a diagram of the Wnt canonical signaling pathway.

### DETAILED DESCRIPTION OF THE DISCLOSURE

### I. Definitions

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. For example, the term “a cell” includes a single cell as well as a plurality or population of cells. Generally, nomenclatures utilized in connection with, and techniques of, cell and tissue culture, molecular biology, and protein and oligonucleotide or polynucleotide chemistry and hybridization described herein are those well-known and commonly used in the art (see, e.g., Green and Sambrook, 2012).

As used herein, the term “polypeptide” refers to a molecule having a sequence of natural and/or unnatural amino acids connected through peptide bonds. The term “peptide” refers to a short polypeptide, typically no more than 30 amino acids long. The amino acid sequence of a polypeptide is referred to as its “primary structure.” The term “protein” refers to a polypeptide having a secondary, tertiary and/or quaternary structure, e.g., structures stabilized by hydrogen bonds, relationships between secondary structures and structures formed of more than one protein. Proteins can be further modified by other attached moieties such as carbohydrate (glycoproteins), lipids (lipoproteins) phosphate groups (phosphoproteins) and the like.

As used herein, an amino acid sequence “consists of” only the amino acids in that sequence.

As used herein, a first amino acid sequence “consists essentially of” a second amino acid sequence if the first amino acid sequence (1) comprises the second amino sequence and (2) is no more than 1, no more than 2 or no more than 3 amino acids longer than the second amino acid sequence.

As used herein, a first amino acid sequence is a “fragment” of a second amino acid sequence if the second amino acid sequence comprises the first amino acid sequence. In certain embodiments, a first amino acid sequence that is a fragment of a second amino acid sequence may have no more than any of 1, 2, 3, 4, 5, 6, 7, 8, 9 or 10 fewer amino acids than the second amino acid sequence.

As used herein, a “functional equivalent” of a reference amino acid sequence is a sequence that is not identical to the reference sequence, but that contains minor alterations such as, for example, insertion, deletion or substitution of one or a few amino acids. A functionally equivalent sequence retains the function (e.g., immunogenicity) of the reference sequence to which it is equivalent. If a functionally equivalent amino acid sequence contains substitution of one or more amino acids with respect to the reference sequence, these will generally be conservative amino acid substitutions.

As used herein, a “conservative amino acid substitution” is one in which one amino acid residue is replaced with another amino acid residue without abolishing the protein's desired properties. Suitable conservative amino acid substitutions can be made by substituting amino acids with similar hydrophobicity, polarity, and R-chain length for one another. See, e.g., Watson, et al., “Molecular Biology of the Gene,” 4th Edition, 1987, The Benjamin/Cummings Pub. Co., Menlo Park, Calif., p. 224. Examples of conservative amino acid substitution include the following (Note, some categories are not mutually exclusive):

Conservative Substitutions

| Type of Amino Acid | Substitutable Amino Acids |
|---|---|
| Hydrophilic | Ala, Pro, Gly, Glu, Asp, Gln, Asn, Ser, Thr |
| Sulphydryl | Cys |
| Aliphatic (non-polar, | Ala, Val, Ile, Leu, Met, Gly, Pro |
| hydrophobic) | |
| Basic | Lys, Arg, His |
| Aromatic | Phe, Tyr, Trp |

As used herein, the term “substantially identical” refers to identity between a first amino acid sequence that contains a sufficient or minimum number of amino acid residues that are i) identical to, or ii) conservative substitutions of aligned amino acid residues in a second amino acid sequence such that the first and second amino acid sequences have a common structural domain and/or common functional activity and/or common immunogenicity. For example, amino acid sequences that contain a common structural or antigenic domain having at least about 90%, 91%, 92%, 93%, 94%, 95%, 96%, 97%, 98% or 99% identity are termed sufficiently or substantially identical. In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity, or encode polypeptides having the same immunogenic properties.

As used herein, the terms “antigen,” “immunogen,” and “antibody target,” refer to a molecule, compound, or complex that is recognized by an antibody, i.e., can be bound by the antibody. The term can refer to any molecule that can be recognized by an antibody, e.g., a polypeptide, polynucleotide, carbohydrate, lipid, chemical moiety, or combinations thereof (e.g., phosphorylated or glycosylated polypeptides, etc.). One of skill will understand that the term does not indicate that the molecule is immunogenic in every context, but simply indicates that it can be targeted by an antibody.

As used herein, the term “epitope” refers to the localized site on an antigen that is recognized and bound by an antibody. Epitopes can include a few amino acids or portions of a few amino acids, e.g., 5 or 6, or more, e.g., 20 or more amino acids, or portions of those amino acids. In some cases, the epitope includes non-protein components, e.g., from a carbohydrate, nucleic acid, or lipid. In some cases, the epitope is a three-dimensional moiety. Thus, for example, where the target is a protein, the epitope can be comprised of consecutive amino acids, or amino acids from different parts of the protein that are brought into proximity by protein folding (e.g., a discontinuous epitope).

As used herein, the term “antibody” refers to an immunoglobulin that recognizes and specifically binds to a one or more target antigen(s), such as a protein, polypeptide, peptide, carbohydrate, polynucleotide, lipid or combinations thereof. This binding occurs through at least one antigen recognition site within the variable region of the immunoglobulin at one or more epitopes on the antigen. The variable region is most critical in binding specificity and affinity. As used herein, the term “antibody” encompasses intact polyclonal antibodies, intact monoclonal antibodies, antibody fragments, single chain Fv (scFv) mutants, multispecific antibodies, chimeric antibodies, humanized antibodies, human antibodies, hybrid antibodies, fusion proteins and any other immunoglobulin molecule comprising an antigen recognition site so long as the antibody exhibit the desired biological activity. Antibodies can be of (i) any of the five major classes of immunoglobulins, based on the identity of their heavy-chain constant domains—alpha (IgA), delta (IgD), epsilon (IgE), gamma (IgG) and mu (IgM), or (ii) subclasses (isotypes) thereof (E.g., IgG1, IgG2, IgG3, IgG4, IgA1 and IgA2). The light chains can be either lambda or kappa. Antibodies can be naked or conjugated to other molecules such as toxins, drugs, radioisotopes, chemotherapeutic agents, etc.

In one embodiment, an “intact antibody” comprises a tetramer composed of two identical pairs of polypeptide chains, each pair having one “light” (about 25 kD) and one “heavy” chain (about 50-70 kD). The heavy chain and light chains are connected through covalent and non-covalent bonds (e.g., disulfide linkage) that vary in number and amount between the various immunoglobulin classes. In one embodiment, each chain comprises a variable region and a constant region. The antigen recognition site of the variable region is composed of hypervariable regions or complementarity determining regions (CDRs) and frameworks regions. The framework regions typically do not come into contact with the antigen but provide structural support for the CDRs. The constant region interacts with other immune cells of the body. Between the constant and variable region (IgG, IgD, IgA only but not IgM or IgE) is the hinge region in the center between the two heavy chains that provides flexibility to articulate antigen binding.

The following are a non-exhaustive list of different antibody forms, all retaining antigen binding activity:

(1) whole immunoglobulins (also referred to as “intact” antibodies) (two light chains and two heavy chains, e.g., a tetramer).

(2) an immunoglobulin polypeptide (a light chain or a heavy chain).

(3) an antibody fragment, such as Fv (a monovalent or bi-valent variable region fragment, and can encompass only the variable regions (e.g., V_{L} and/or V_{H}), Fab (V_{L}C_{L} V_{H}C_{H}), F(ab′)2, Fv (V_{L}V_{H}), scFv (single chain Fv) (a polypeptide comprising a V_{L} and V_{H} joined by a linker, e.g., a peptide linker), (scFv)2, sc(Fv)2, bispecific sc(Fv)2, bispecific (scFv)2, minibody (sc(FV)2 fused to C_{H}3 domain), triabody is trivalent sc(Fv)3 or trispecific sc(Fv)3.

(4) a multivalent antibody (an antibody comprising binding regions that bind two different epitopes or proteins, e.g., “scorpion” antibody.

(5) a fusion protein comprising a binding portion of an immunoglobulin fused to another amino acid sequence (such as a fluorescent protein).

As used herein, the term “antibody fragment” refers to a part or portion of an antibody or antibody chain comprising fewer amino acid residues than an intact or complete antibody or antibody chain and which binds the antigen or competes with intact antibody. Fragments can be obtained via chemical or enzymatic treatment of an intact or complete antibody or antibody chain. Fragments can also be obtained by recombinant means. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be constructed by recombinant expression techniques.

While various antibody fragments are defined in terms of products of the digestion of an intact antibody, one of skill will appreciate that such fragments may also be synthesized de novo chemically or constructed and expressed using recombinant DNA methodology.

A single chain Fv (scFv) refers to a polypeptide comprising a V_{L} and V_{H} joined by a linker, e.g., a peptide linker. ScFvs can also be used to form tandem (or di-valent) scFvs or diabodies. Production and properties of tandem scFvs and diabodies are described, e.g., in Asano et al. (2011) J Biol. Chem. 286:1812; Kenanova et al. (2010) Prot Eng Design Sel 23:789; Asano et al. (2008) Prot Eng Design Sel 21:597.

Antibody fragments further include Fd (the portion of the heavy chain included in the Fab fragment) and single domain antibodies. A single domain antibody (sdAb) is a variable domain of either a heavy chain or a light chain, produced by recombinant methods.

The phrase “CDR sequence set” as used herein refers to the 3 heavy chain and/or 3 light chain CDRs of a particular antibody described herein. A “light chain” CDR sequence set refers to the light chain CDR sequences. A “heavy chain” CDR sequence set refers to the heavy chain CDR sequences. A “full” CDR sequence set refers to both heavy chain and light chain CDR sequences. For example, for antibody 5017, as shown in Table 1a, the full CDR sequence set comprises or consists of SVSSA (CDR L1), SASSLYS (CDR L2) AAYHWPPLF (CDR L3), LYYTDM (CDR H1), SISLFFGYVS (CDR H2) AND YLAM (CDR H3). The CDR sequence for each CDR can, for example, comprise, consist essentially of, or consist of the CDR in Table 1a or 3a. CDRs are predicted based on IMGT sequence alignment.

As used herein, the term “monoclonal antibody” refers to a clonal preparation or composition of antibodies with a single binding specificity and affinity for a given epitope on an antigen (“monoclonal antibody composition”). A “polyclonal antibody” refers to a preparation or composition of antibodies that are raised against a single antigen, but with different binding specificities and affinities (“polyclonal antibody composition”).

As used herein, the term “chimeric antibody” refers to an antibody having amino acid sequences derived from two or more species. In one embodiment, the variable region of both light and heavy chains correspond to the variable region of antibodies derived from one species of mammal (e.g., mouse, rat, rabbit, etc.) with the desired specificity, affinity and capability, while the constant region are homologous the sequence derived from another species (typically in the subject receiving the therapy, e.g., human) to avoid eliciting an immune response.

As used herein, the term “humanized antibody” refers to a chimeric antibody in which the CDRs, obtained from the VH and VL regions of a non-human antibody having the desired specificity, affinity and capability are grafted to a human framework sequence. In one embodiment, the framework residues of the humanized antibody are modified to refine and optimize the antibody specificity, affinity and capability. Humanization, i.e., substitution of non-human CDR sequences for the corresponding sequences of a human antibody, can be performed following the methods described in, e.g., U.S. Pat. Nos. 5,545,806; 5,569,825; 5,633,425; 5,661,016; Riechmann et al., Nature 332:323-327 (1988); Marks et al., Bio/Technology 10:779-783 (1992); Morrison, Nature 368:812-13 (1994); Fishwild et al., Nature Biotechnology 14:845-51 (1996).

As used herein, the term “human antibody” refers to an antibody produced by a human or an antibody having an amino acid sequence corresponding thereto made by any technique known in the art.

As used herein, the term “hybrid antibody” refers to antibody in which pairs of heavy and light chains form antibodies with different antigenic determinant regions are assembled together so that two different epitopes or two different antigens can be recognized and bound by the resulting tetramer. Hybrid antibodies can be bispecific (binding 2 distinct antigens or epitopes) or multispecific (>1 distinct antigen or epitope).

As used herein, an antibody is “monospecific” if all of its antigen binding sites bind to the same epitope.

As used herein, an antibody is “bispecific” if it has at least two different antigen binding sites which each bind to a different epitope or antigen.

As used herein, an antibody is “polyvalent” if it has more than one antigen binding site. For example, an antibody that is tetravalent has four antigen binding sites.

The specificity of the binding can be defined in terms of the comparative dissociation constants (K_{d}) of the antibody (or other targeting moiety) for target, as compared to the dissociation constant with respect to the antibody and other materials in the environment or unrelated molecules in general. A larger (higher) K_{d} is a K_{d} that describes a lower affinity interaction. Conversely a smaller (lower) K_{d} is a K_{d} that describes a higher affinity interaction or tighter binding. By way of example only, the K_{d} for an antibody specifically binding to a target may be femtomolar, picomolar, nanomolar, or micromolar and the K_{d} for the antibody binding to unrelated material may be millimolar or higher. Binding affinity can be in the micromolar range (kD=10⁻⁴ to 10^{-l}, nanomole range (kD=10⁻⁷ M to 10⁻⁹ M), picomole range (kD=10⁻¹⁹ M to 10⁻¹² M), or femtomole range (kD=10⁻¹³ M to 10⁻¹⁵ M).

As used herein, an antibody “binds” or “recognizes” an antigen or epitope if it binds the antigen or epitope with a Kd of less than 10⁻⁴M (i.e., in the micromolar range). The term “binds” with respect to a cell type (e.g., an antibody that binds cancer cells), typically indicates that an agent binds a majority of the cells in a pure population of those cells. For example, an antibody that binds a given cell type typically binds to at least ⅔ of the cells in a population of the indicated cells (e.g., 67, 75, 80, 85, 90, 91, 92, 93, 94, 95, 96, 97, 98, 99, or 100%). In some cases, binding to a polypeptide can be assayed by comparing binding of the antibody to a cell that presents the polypeptide to binding (or lack thereof) of the antibody to a cell that does not express the polypeptide. One of skill will recognize that some variability will arise depending on the method and/or threshold of determining binding. Affinity of an antibody for a target can be determined according to methods known in the art, e.g., as reviewed in Ernst et al. Determination of Equilibrium Dissociation Constants, Therapeutic Monoclonal Antibodies Miley & Sons ed. 2009).

As used herein, the term “greater affinity” as used herein refers to a relative degree of antibody binding where an antibody X binds to target Y more strongly (Kₒₙ) and/or with a smaller dissociation constant (K_{off}) than to target Z, and in this context antibody X has a greater affinity for target Y than for Z. Likewise, the term “lesser affinity” herein refers to a degree of antibody binding where an antibody X binds to target Y less strongly and/or with a larger dissociation constant than to target Z, and in this context antibody X has a lesser affinity for target Y than for Z. The affinity of binding between an antibody and its target antigen, can be expressed as K_{A} equal to 1/K_{D} where K_{D} is equal to kₒₙ/k_{off}. The kₒₙ and k_{off} values can be measured using surface plasmon resonance technology, for example, using a Molecular Affinity Screening System (MASS-1) (Sierra Sensors GmbH, Hamburg, Germany). An antagonist or blocking antibody is an antibody that partially or fully blocks inhibits or neutralizes a biological activity related to the target antigen relative to the activity under similar physiological conditions when the antibody is not present. Antagonists can be competitive, non-competitive or irreversible. A competitive antagonist is a substance that binds to a natural ligand or receptor at the same site as the natural ligand-receptor interaction or binds allosterically in a manner that induces a change to prevent normal binding. A non-competitive antagonist binds at a different site than the natural ligand-receptor interaction, but lowers the KD or signal resulting from the interaction. An irreversible inhibitor causes covalent modifications to the receptor preventing any subsequent binding.

As used herein, the term “avidity” refers to the overall stability of the binding complex between the antibody and the target antigen. It is governed by three factors, (i) the intrinsic affinity of the antibody for the antigen, (2) the valency of the antibody, and (3) the geometric arrangement of the interacting components. Affinity is the strength of the interaction between the antibody and a single target, whereas avidity is an accumulated strength of multiple affinities. In one embodiment, the antibodies disclosed herein are divalent.

As used herein, an antibody “preferentially binds” binds a first antigen relative to a second antigen if it binds the first antigen with greater affinity than it does the second antigen. Preferential binding can be at least any of 2-fold, 5-fold, 9-fold, 10-fold, 20-fold, 30-fold, 40-fold, 50-fold, 100-fold, 500-fold or 1000-fold greater affinity. So, for example, an antibody preferentially binds a first FZD protein relative to a second FZD protein if it binds the first FZD protein with greater affinity than it binds the second FZD protein.

As used herein, an antibody “specifically binds” or is “specific for” a target antigen or target group of antigens if it binds the target antigen or each member of the target group of antigens with an affinity of at least any of 1×10⁻⁶ M, 1×10⁻⁷M, 1×10⁻⁸M, 1×10⁻⁹ M, 1×10⁻¹⁰ M, 1×10⁻¹¹ M, 1×10⁻¹² M, and, for example, binds to the target antigen or each member of the target group of antigens with an affinity that is at least two-fold greater than its affinity for non-target antigens to which it is being compared. Typically, specific binding is characterized by binding the antigen with sufficient affinity that the antibody is useful as a diagnostic to detect the antigen or epitope and/or as a therapeutic agent in targeting the antigen or epitope.

If an antibody specifically binds a target group of proteins (e.g., some or all members of the Frizzled protein family), then the binding affinity of the antibody for the member of the target group to which it binds most weakly is greater than the binding affinity of the antibody for non-target antigens. In one embodiment, an antibody that specifically binds a cysteine rich domain (CRD) of each of one or a plurality of human Frizzled (FZD) receptors selected from FZD 1, 2, 4, 5, 7, 8, 9 means an antibody specifically binds the selected members of this group as compared with non-selected group members or other antigens, more generally. So, for example, an antibody that specifically binds a cysteine rich domain of the target group consisting of FZD1, FZD2, FZD4, FZD5, and FZD7, specifically binds these proteins and not FZD3, FZD8, FZD9, and FZD10.

As used herein, and antibody “blocks” or “antagonizes” the binding of a ligand to a receptor when it competitively reduces or prevents interaction all of the ligand with the receptor. In an embodiment, the measured level of reduction can be at least any of 5%, 10%, 25%, 50%, 80%, 90%, 95%, 97.5%, 99%, 99.5%, 99.9% of a control (e.g., untreated) cell. For example, an antibody that antagonizes or blocks the binding of a Wnt ligand to a FZD receptor competitively reduces or prevents the interaction of a Wnt protein with FZD receptor. This results in attenuation or blocking of a downstream signaling event associated with Wnt signaling. This includes, for example, activation of disheveled, dissolution of the β-catenin destructive complex, lower cytosolic levels of β-catenin, and/or lower activity of TCF/LEF-mediated transcription.

The term “captures” with respect to an antibody target (e.g., antigen, analyte, immune complex), typically indicates that an antibody binds a majority of the antibody targets in a pure population (assuming appropriate molar ratios). For example, an antibody that binds a given antibody target typically binds to at least ⅔ of the antibody targets in a solution (e.g., at least any of 67, 75, 80, 85, 90, 91, 92, 93, 94, 95, 96, 97, 98, 99, or 100%). One of skill will recognize that some variability will arise depending on the method and/or threshold of determining binding.

The term “conjugate” refers to a first molecule, e.g., an antibody (an “immunoconjugate”), chemically coupled with a moiety, such as a detectable label or a biologically active moiety, such as a drug, toxin or chemotherapeutic or cytotoxic agent. Accordingly, this disclosure contemplates antibodies conjugated with one or more moieties. Furthermore, an antibody can be “conjugated antibody” or a “non-conjugated antibody” (that is, not conjugated with a moiety.

As used herein, the term “antibody-drug conjugate” or (“ADC”) refers to an antibody conjugated with a drug. Typically, conjugation involves covalent binding through a linker.

As used herein, the term “labeled” molecule (e.g., nucleic acid, protein, or antibody) refers to a molecule that is bound to a detectable label, either covalently, through a linker or a chemical bond, or noncovalently, through ionic, van der Waals, electrostatic, or hydrogen bonds, such that the presence of the molecule may be detected by detecting the presence of the detectable label bound to the molecule.

As used herein, the term “detectable label” refers to a composition detectable by spectroscopic, photochemical, biochemical, immunochemical, chemical, or other physical means. Examples of detectable labels are described herein and include, without limitation, colorimetric, fluorescent, chemiluminescent, enzymatic, and radioactive labels. For the purposes of the present disclosure, a detectable label can also be a moiety that does not itself produce a signal (e.g., biotin), but that binds to a second moiety that is able to produce a signal (e.g., labeled avidin).

The term “cross-linked” with respect to an antibody refers to attachment of the antibody to a solid or semisolid matrix (e.g., sepharose, beads, microtiter plate), or to another protein or antibody. For example, an antibody can be multimerized to create an antibody complex with multiple (more than 2) antigen-binding sites. The antibody can be multimerized by expressing the antibody as a high-valency isotype (e.g., IgA or IgM, which typically form complexes of 2 or 5 antibodies, respectively). Antibody multimerization can also be carried out by using a cross-linker comprising a reactive group capable of linking proteins (e.g., carbodiimide, NHS esters, etc.). Methods and compositions for cross-linking an antibody to a matrix are described, e.g., in the Abcam and New England Biolab catalogs and websites (available at abcam.com and neb.com). Cross-linker compounds with various reactive groups are described, e.g., in Thermo Fisher Scientific catalog and website (available at piercenet.com).

As used herein, the term “immunoassay” refers to a method for detecting an analyte by detecting binding between an antibody that recognizes the analyte and the analyte.

As used herein, the term “expression construct” refers to a polynucleotide comprising an expression control sequence operatively linked with a heterologous nucleotide sequence (i.e., a sequence to which the expression control sequence is not normally connected to in nature) that is to be the subject of expression. As used herein, the term “expression vector” refers to a polynucleotide comprising an expression construct and sequences sufficient for replication in a host cell or insertion into a host chromosome. Plasmids and viruses are examples of expression vectors. As used herein, the term “expression control sequence” refers to a nucleotide sequence that regulates transcription and/or translation of a nucleotide sequence operatively linked thereto. Expression control sequences include promoters, enhancers, repressors (transcription regulatory sequences) and ribosome binding sites (translation regulatory sequences).

The term “vector” as used herein comprises any intermediary vehicle for a nucleic acid molecule which enables said nucleic acid molecule, for example, to be introduced into prokaryotic and/or eukaryotic cells and/or integrated into a genome, and include plasmids, phagemids, bacteriophages or viral vectors such as retroviral based vectors, Adeno Associated viral vectors and the like. The term “plasmid” as used herein generally refers to a construct of extrachromosomal genetic material, usually a circular DNA duplex, which can replicate independently of chromosomal DNA.

As used herein, a nucleotide sequence is “operatively linked” with an expression control sequence when the expression control sequence functions in a cell to regulate transcription of the nucleotide sequence. This includes promoting transcription of the nucleotide sequence through an interaction between a polymerase and a promoter.

As used herein, a “host cell” refers to a recombinant cell comprising an expression construct.

As used herein, the term “biological sample” refers to a sample containing cells (e.g., tumor cells) or biological molecules derived from cells. A biological sample can be obtained from a subject, e.g., a patient, from an animal, such as an animal model, or from cultured cells, e.g., a cell line or cells removed from a patient and grown in culture for observation. A biological sample can comprise tissue and/or liquid. It can be obtained from any biological source including without limitation blood, a blood fraction (e.g., serum or plasma), cerebrospinal fluid (CSF), lymph, tears, saliva, sputum, buccal swab, milk, urine or feces. A biological sample can be a biopsy, such as a tissue biopsy, such as needle biopsy, fine needle biopsy, surgical biopsy, etc. The sample can comprise a tissue sample harboring a lesion or suspected lesion, although the biological sample may also be derived from another site, e.g., a site of suspected metastasis, a lymph node, or from the blood. A biological sample can be a fraction of a sample taken from a subject. An example of a tissue sample includes a brain tissue sample or a nerve tissue sample. Methods of obtaining such biological samples are known in the art including but not limited to standard blood retrieval procedures.

As used herein, the term “diagnosis” refers to a relative probability that a subject has a disorder such as cancer. Similarly, the term “prognosis” refers to a relative probability that a certain future outcome may occur in the subject. For example, in the context of the present disclosure, prognosis can refer to the likelihood that an individual will develop cancer, have recurrence, that the cancer will metastasize, that the cancer will be cured, or the likely severity of the disease (e.g., severity of symptoms, rate of functional decline, survival, etc.). The terms are not intended to be absolute, as will be appreciated by any one of skill in the field of medical diagnostics.

As used herein, the term terms “therapy,” “treatment,” “therapeutic intervention” and “amelioration” refer to any activity resulting in a reduction in the severity of symptoms. In the case of cancer, treatment can refer to, e.g., reducing tumor size, number of cancer cells, growth rate, metastatic activity, reducing cell death of non-cancer cells, reduced nausea and other chemotherapy or radiotherapy side effects, etc. The terms “treat” and “prevent” are not intended to be absolute terms. Treatment and prevention can refer to any delay in onset, amelioration of symptoms, improvement in patient survival, increase in survival time or rate, etc. Treatment and prevention can be complete (undetectable levels of neoplastic cells) or partial, such that fewer neoplastic cells are found in a patient than would have occurred without the present intervention. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects, the severity of disease is reduced by at least 25%, 50%, 75%, 80%, or 90%, or in some cases, no longer detectable using standard diagnostic techniques.

As used herein, the terms “effective amount,” “effective dose,” and “therapeutically effective amount,” refer to an amount of an agent, such as an antibody or immunoconjugate, that is sufficient to generate a desired response, such as reduce or eliminate a sign or symptom of a condition or ameliorate a disorder. In some examples, an “effective amount” is one that treats (including prophylaxis) one or more symptoms and/or underlying causes of any of a disorder or disease and/or prevents progression of a disease. For example, for the given parameter, a therapeutically effective amount will show an increase or decrease of therapeutic effect at least any of 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least any of a 1.2-fold, 1.5-fold, 2-fold, 5-fold, or more effect over a control.

As used herein, the term “pharmaceutical composition” refers to a composition comprising a pharmaceutical compound (e.g., a drug) and a pharmaceutically acceptable carrier.

As used herein, the term “pharmaceutically acceptable” refers to a carrier that is compatible with the other ingredients of a pharmaceutical composition and can be safely administered to a subject. The term is used synonymously with “physiologically acceptable” and “pharmacologically acceptable”. Pharmaceutical compositions and techniques for their preparation and use are known to those of skill in the art in light of the present disclosure. For a detailed listing of suitable pharmacological compositions and techniques for their administration one may refer to texts such as Remington's Pharmaceutical Sciences, 17th ed. 1985; Brunton et al., “Goodman and Gilman's The Pharmacological Basis of Therapeutics,” McGraw-Hill, 2005; University of the Sciences in Philadelphia (eds.), “Remington: The Science and Practice of Pharmacy,” Lippincott Williams & Wilkins, 2005; and University of the Sciences in Philadelphia (eds.), “Remington: The Principles of Pharmacy Practice,” Lippincott Williams & Wilkins, 2008.

Pharmaceutically acceptable carriers will generally be sterile, at least for human use. A pharmaceutical composition will generally comprise agents for buffering and preservation in storage, and can include buffers and carriers for appropriate delivery, depending on the route of administration. Examples of pharmaceutically acceptable carriers include, without limitation, normal (0.9%) saline, phosphate-buffered saline (PBS) Hank's balanced salt solution (HBSS) and multiple electrolyte solutions such as PlasmaLyte ATM (Baxter).

Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Pluronics, Triton-X, or polyethylene glycol (PEG).

The terms “dose” and “dosage” are used interchangeably herein. A dose refers to the amount of active ingredient given to an individual at each administration. For the present invention, the dose can refer to the concentration of the antibody or associated components, e.g., the amount of therapeutic agent or dosage of radiolabel. The dose will vary depending on a number of factors, including frequency of administration; size and tolerance of the individual; severity of the condition; risk of side effects; the route of administration; and the imaging modality of the detectable label (if present). One of skill in the art will recognize that the dose can be modified depending on the above factors or based on therapeutic progress. The term “dosage form” refers to the particular format of the pharmaceutical, and depends on the route of administration. For example, a dosage form can be in a liquid, e.g., a saline solution for injection.

As used herein, the term “subject” refers to an individual animal. The term “patient” as used herein refers to a subject under the care or supervision of a health care provider such as a doctor or nurse. Subjects include mammals, such as humans and non-human primates, such as monkeys, as well as dogs, cats, horses, bovines, rabbits, rats, mice, goats, pigs, and other mammalian species. Subjects can also include avians. A patient can be an individual that is seeking treatment, monitoring, adjustment or modification of an existing therapeutic regimen, etc. The term “cancer subject” refers to an individual that has been diagnosed with cancer. Cancer patients can include individuals that have not received treatment, are currently receiving treatment, have had surgery, and those that have discontinued treatment.

In the context of treating cancer, a subject in need of treatment can refer to an individual that has cancer or a pre-cancerous condition, has had cancer and is at risk of recurrence, is suspected of having cancer, is undergoing standard treatment for cancer, such as radiotherapy or chemotherapy, etc.

“Cancer”, “tumor,” “transformed” and like terms include precancerous, neoplastic, transformed, and cancerous cells, and can refer to a solid tumor, or a non-solid cancer (see, e.g., Edge et al. AJCC Cancer Staging Manual (7th ed. 2009); Cibas and Ducatman Cytology: Diagnostic principles and clinical correlates (3rd ed. 2009)). Cancer includes both benign and malignant neoplasms (abnormal growth). “Transformation” refers to spontaneous or induced phenotypic changes, e.g., immortalization of cells, morphological changes, aberrant cell growth, reduced contact inhibition and anchorage, and/or malignancy (see, Freshney, Culture of Animal Cells a Manual of Basic Technique (3rd ed. 1994)). Although transformation can arise from infection with a transforming virus and incorporation of new genomic DNA, or uptake of exogenous DNA, it can also arise spontaneously or following exposure to a carcinogen.

The term “cancer” can refer to any cancer, including without limitation, leukemias, carcinomas, sarcomas, adenocarcinomas, lymphomas, solid and lymphoid cancers, etc. Examples of different types of cancer include, but are not limited to, lung cancer (e.g., non-small cell lung cancer or NSCLC), breast cancer, prostate cancer, colorectal cancer, bladder cancer, ovarian cancer, leukemia, liver cancer (i.e., hepatocarcinoma), renal cancer (i.e., renal cell carcinoma), thyroid cancer, pancreatic cancer, uterine cancer, cervical cancer, testicular cancer, esophageal cancer, stomach (gastric) cancer, kidney cancer, cancer of the central nervous system, skin cancer, glioblastoma and melanoma.

As used herein, a chemical entity, such as a polypeptide, is “substantially pure” if it is the predominant chemical entity of its kind (e.g., of polypeptides) in a composition. This includes the chemical entity representing more than 50%, more than 80%, more than 90%, more than 95%, more than 98%, more than 99%, more than 99.5%, more than 99.9%, or more than 99.99% of the chemical entities of its kind in the composition.

The phrase “isolated antibody” refers to antibody produced in vivo or in vitro that has been removed from the source that produced the antibody, for example, an animal, hybridoma or other cell line (such as recombinant insect, yeast or bacterial cells that produce antibody).

“Substantially pure” or “isolated” means an object species is the predominant species present (i.e., on a molar basis, more abundant than any other individual macromolecular species in the composition), and a substantially purified fraction is a composition wherein the object species comprises at least about 50% (on a molar basis) of all macromolecular species present. Generally, a substantially pure composition means that about 80% to 90% or more of the macromolecular species present in the composition is the purified species of interest. The object species is purified to essential homogeneity (contaminant species cannot be detected in the composition by conventional detection methods) if the composition consists essentially of a single macromolecular species. Solvent species, small molecules (<500 Daltons), stabilizers (e.g., BSA), and elemental ion species are not considered macromolecular species for purposes of this definition.

The term “sequence identity” as used herein refers to the percentage of sequence identity between two polypeptide sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present application. BLAST protein searches can be performed with the XBLAST program parameters set, e.g., to score-50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

For antibodies, percentage sequence identities can be determined when antibody sequences maximally aligned by IMGT. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, multiplied by 100 to convert to percentage.

Percent amino acid sequence identity may also be determined using the sequence comparison program NCBI-BLAST2 (Altschul et al., Nucleic Acids Res. 25:3389-3402 (1997)). The NCBI-BLAST2 sequence comparison program may be obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the amino acid sequence identity of A to B will not equal the % amino acid sequence identity of B to A. The term “nucleic acid sequence” as used herein refers to a sequence of nucleoside or nucleotide monomers consisting of naturally occurring bases, sugars and intersugar (backbone) linkages and includes cDNA. The term also includes modified or substituted sequences comprising non-naturally occurring monomers or portions thereof. The nucleic acid sequences of the present application may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. It is understood that polynucleotides comprising non-transcribable nucleotide bases may be useful as probes in, for example, hybridization assays. The nucleic acid can be either double stranded or single stranded, and represents the sense or antisense strand. Further, the term “nucleic acid” includes the complementary nucleic acid sequences as well as codon optimized or synonymous codon equivalents.

The term “isolated nucleic acid” as used herein refers to a nucleic acid substantially free of cellular material or culture medium when produced by recombinant DNA techniques, or chemical precursors, or other chemicals when chemically synthesized. An isolated nucleic acid is also substantially free of sequences that naturally flank the nucleic acid (i.e. sequences located at the 5′ and 3′ ends of the nucleic acid) from which the nucleic acid is derived.

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g., 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.−16.6 (Log 10 [Na+])+0.41 (%(G+C)−600/1), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example, if nucleic acid molecules are sought that have a >95% identity, the final wash temperature will be reduced by about 5° C. Based on these considerations those skilled in the art will be able to readily select appropriate hybridization conditions. In preferred embodiments, stringent hybridization conditions are selected. By way of example the following conditions may be employed to achieve stringent hybridization: hybridization at 5× sodium chloride/sodium citrate (SSC)/5×Denhardt's solution/1.0% SDS at Tm—5° C. based on the above equation, followed by a wash of 0.2×SSC/0.1% SDS at 60° C. Moderately stringent hybridization conditions include a washing step in 3×SSC at 42° C. It is understood, however, that equivalent stringencies may be achieved using alternative buffers, salts and temperatures. Additional guidance regarding hybridization conditions may be found in: Current Protocols in Molecular Biology, John Wiley & Sons, N.Y., 2002, and in: Sambrook et al., Molecular Cloning: a Laboratory Manual, Cold Spring Harbor Laboratory Press, 2001.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. For example, a subject with cancer can be treated to prevent progression can be treated with an antibody, immunoconjugate, nucleic acid or composition described herein to prevent progression.

As used herein, the term “administration” means to provide or give a subject an agent, such as a composition comprising an effective amount of an antibody by an effective route such as an intratumor or an intravenous administration route.

As used herein, the term “diluent” refers to a pharmaceutically acceptable carrier which does not inhibit a physiological activity or property of an active compound, such as an antibody, or immunoconjugate, to be administered and does not irritate the subject and does not abrogate the biological activity and properties of the administered compound. Diluents include any and all solvents, dispersion media, coatings, surfactants, antioxidants, preservative salts, preservatives, binders, excipients, disintegration agents, lubricants, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

Compositions or methods “comprising” or “including” one or more recited elements may include other elements not specifically recited. For example, a composition that “comprises” or “includes” an antibody may contain the antibody alone or in combination with other ingredients.

In understanding the scope of the present disclosure, the term “consisting” and its derivatives, as used herein, are intended to be close ended terms that specify the presence of stated features, elements, components, groups, integers, and/or steps, and also exclude the presence of other unstated features, elements, components, groups, integers and/or steps.

The recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.” Further, it is to be understood that the singular forms of the articles “a,” “an,” and “the” include plural references unless the context clearly dictates otherwise. For example, the term “an antibody” or “at least one antibody” can include a plurality of antibodies, including mixtures thereof.

The terms “Frizzled” and “FZD” refer, depending on context, to any gene or protein member of the Frizzled family. Frizzled proteins are involved in the activation of Disheveled protein in the cytosol. Frizzled refers to any of Frizzled-1, Frizzled-2, Frizzled-3, Frizzled-4, Frizzled-5, Frizzled-6, Frizzled-7, Frizzled-8, Frizzled-9 and Frizzled-10. Frizzled 4 (“FZD4”) (also referred to as CD344, EVR1, FEVR, FZD4S, Fz-4, Fz4, FzE4, GPCR, hFz4, and frizzled class receptor 4), is a member of the frizzled gene family of proteins. The gene has ENTREZ Gene ID: 8322. The protein has NCBI Reference Sequence: NP_036325.2.

“Lipoprotein receptor-related proteins”, “low density lipoprotein receptor-related proteins” (HGNC) or “prolow-density lipoprotein receptor-related protein” (UniProt), abbreviated “LRP”, are a group of genes and proteins. They include: LRP1, LRP1B, LRP2 (megalin), LRP3, LRP4, LRP5, LRP6, LRP8 (apolipoprotein e receptor), LRP10, LRP11, and LRP12. LRP5 and LRP6 are part of the LRP5/LRP6/Frizzled co-receptor group that is involved in canonical Wnt pathway. LRP5 is also known as LRP5, BMND1, EVR1, EVR4, HBM, LR3, LRP-5, LRP7, OPPG, OPS, OPTA1, VBCH2, and LDL receptor related protein 5. The LRP5 gene has ENTREZ Gene ID: 4041 and the protein has NCBI Reference Sequence: NP_002326. The LRP6 gene has ENTREZ Gene ID: 4040 and the protein has NCBI Reference Sequence: NP_002327. LRP6 is also known as ADCAD2, STHAG7.

### II. Disorders Associated with FRZ Signaling Dysregulation

Wnt binding to FZD destabilizes a β-catenin binding complex causing β-catenin degradation. The effect is increased levels of intracellular β-catenin. Accordingly, provided herein are methods of blocking Wnt binding to frizzled proteins, in particular to FZD4, but also to other members of the frizzled family, such as FZD1, FZD2, FZD4, FZD5, FZD7, FZD8 and FZD9.

A “FZD-associated disorder” (e.g., a “FZD4-associated disorder” or a “FZD5-associated disorder”) refers to a conditions or disease correlated with dysregulation of the particular FZD receptor referred to. Dysregulation refers to abnormal signaling that increases normal β-catenin mediated transcriptional changes or any other intracellular signaling pathways governed by these receptors.

Various Frizzled receptors have been associated with various cancers. More specifically, FZD1 has been associated with neuroblastoma. FZD2 has been associated with liver cancer, lung cancer, endometrial cancer, and salivary adenoid cystic carcinoma cancer. FZD3 has been associated with colorectal cancer. FZD4 has been associated with acute myeloid leukemia, prostate cancer, glioblastoma, bladder cancer and cervical cancer. FZD5 has been associated with pancreatic cancer, colon cancer and prostate cancer. FZD6 has been associated with colorectal cancer and breast cancer. FZD7 has been associated with esophageal cancer, glioma, breast cancer, gastric cancer, and colorectal cancer. FZD8 has been associated with prostate cancer, breast cancer, and lung cancer. FZD9 has been associated with astrocytoma, and osteosarcoma. FZD10 has been associated with colorectal cancer and synovial sarcoma.

### III. Anti FZD4 Antibodies

A. Antibodies

Antibodies against Frizzled receptors (FZD) are described herein, including antibodies that bind more than one FZD and others that preferentially bind FZD4. These antibodies bind to the Frizzled receptors, block ligand WNT binding and modulate frizzled receptor signaling. These antibodies also show anti-proliferative effects have therapeutic potential for treating cancer and other diseases where the frizzled receptors are dysregulated.

Accordingly, an aspect of the disclosure includes an isolated antibody that specifically binds a Frizzled receptor (FZD) cysteine rich domain (CRD) comprising a light chain variable region and a heavy chain variable region, the heavy chain variable region comprising complementarity determining regions CDR-H1, CDR-H2 and CDR-H3, the light chain variable region comprising complementarity determining region CDR-L1, CDR-L2 and CDR-L3, and with the amino acid sequences of said CDRs comprising, consisting essentially of, or consisting of sequences selected from sequences in Table 1a or 3a.

In an embodiment, the antibody comprises a CDR sequence set selected from the CDR sequence sets in Table 1a, that is, for clones 5016 to 5037 and 6498 to 6500.

TABLE 1a
CDR Amino Acid sequences for FZD4 antibodies

| Clone | Well | | | | | | |
|---|---|---|---|---|---|---|---|
| ID | ID | CDR L1 | CDR L2 | CDR L3 | CDR H1 | CDR H2 | CDR H3 |
| 5016 | | SVSSA | SASSLYS | GVYLF | IHSSSI | ATYSSFGSIT | YHHPFGYAL |
| 5017 | 4A1 | SVSSA | SASSLYS | AAYHWPPLF | LYYTDM | SISLFFGYVS | YLAM |
| 5018 | 4A2 | SVSSA | SASSLYS | GVYLF | IYFSSI | SNYPSFGSNS | YHFPFAYSL |
| 5019 | 4A3 | SVSSA | SASSLYS | GVYLF | IGSSSI | SIYSAFASTS | YHFPFGFAL |
| 5020 | 4A4 | SVSSA | SASSLYS | GVYLF | VNSSSI | AFYSSFGATS | YHFPFGHAL |
| 5021 | 4A5 | SVSSA | SASSLYS | GVYLF | IGSSSI | AYYSAFASSS | YHYPFGHAL |
| 5022 | 4A6 | SVSSA | SASSLYS | GVYLF | IHFSSI | CSYPSFGSTS | YHYPFGHAL |
| 5014 | 4A7 | SVSSA | SASSLYS | GVYLF | IYSASI | SRYPSFGSTS | YHYPFGTAL |
| 5023 | 4A8 | SVSSA | SASSLYS | GVYLF | IHSSSI | AIYSSFSANS | YHYPFGYAL |
| 6494 | 4A9 | SVSSA | SASSLYS | GVYLF | IYFSSI | SNYPAFGSTS | YHYPFGYAM |
| 5025 | 4A10 | SVSSA | SASSLYS | GVYLF | IYSSSI | SIYSAFLSTT | YHYPHGHAL |
| 6495 | 4A11 | SVSSA | SASSLYS | SSYSLI | LSYSFF | SIYPSSGYTY | PAPFSYHVL |
| 6496 | 4A12 | SVSSA | SASSLYS | SSYSLI | LSFYFL | SIYPYSGYTY | AAPGSYHPM |
| 6497 | 4B1 | SVSSA | SASSLYS | SSYSLI | LSSYYM | SIYPFHASTY | AAPYFYGVM |
| 5027 | 4B2 | SVSSA | SASSLYS | SSYSLI | SSFYFM | TVYPYLDYTY | AFPGSYHPM |
| 5028 | 4B3 | SVSSA | SASSLYS | SSYSLI | LSYYYM | SIYPYSRNTF | AYPFSYHFM |
| 5029 | 4B4 | SVSSA | SASSLYS | SSYSLI | LSYYYM | SIYPFSGYST | PSAFSYHPM |
| 5030 | 4B5 | SVSSA | SASSLYS | SSYSLI | LSFYYM | SIYPYYAYTY | PVAGAYHPM |
| 5031 | 4B6 | SVSSA | SASSLYS | SSYSLI | IASYFT | SIYLSFGYGY | SSLGFYNGM |
| 6498 | 4B7 | SVSSA | SASSLYS | SSYSLI | FSSSSI | CCNSAYRYGP | TVRGSKKPYFSGWAM |
| 6499 | 4B8 | SVSSA | SASSLYS | SSYSLI | LSYYFM | SIYPYAGNTY | TYPGYYYIL |
| 5034 | 4B9 | SVSSA | SASSLYS | WAYGPF | IYYYPM | SFYSYYSFTY | SGVGGDHAL |
| 5035 | 4B10 | SVSSA | SASSLYS | YYHPI | FSAYNI | SLYTSYGYTY | VWYVVQ |
| 5036 | 4B11 | SVSSA | SASSLYS | YYSLF | FSSSSI | YIYPFNGYSY | GYFYTWGGM |
| 5037 | 4B12 | SVSSA | SASSLYS | YYSLF | FSSSSI | YIYPSYDYTY | GYFYTWGGM |
| 6500 | 4C1 | SVSSA | SASSLYS | YYSLF | IYYFGM | YISPPYGFTY | GYYYSWGGM |

TABLE 1b
CDR Nucleic Acid sequences for FZD4 antibodies

| Clone | Well | | | |
|---|---|---|---|---|
| ID | ID | CDR L1 | CDR L2 | CDR L3 |
| 5016 | | TCCGTGTCCAGCGCT | TCGGCATCCAGCCTCTACTCT | GGTGTTTACCTGTTC |
| 5017 | 4A1 | TCCGTGTCCAGCGCT | TCGGCATCCAGCCTCTACTCT | GCTGCTTACCATTGGCCGCCG |
| | | | | CTGTTCACG |
| 5018 | 4A2 | TCCGTGTCCAGCGCT | TCGGCATCCAGCCTCTACTCT | GGTGTTTACCTGTTCACG |
| 5019 | 4A3 | TCCGTGTCCAGCGCT | TCGGCATCCAGCCTCTACTCT | GGTGTTTACCTGTTCACG |
| 5020 | 4A4 | TCCGTGTCCAGCGCT | TCGGCATCCAGCCTCTACTCT | GGTGTTTACCTGTTCACG |
| 5021 | 4A5 | TCCGTGTCCAGCGCT | TCGGCATCCAGCCTCTACTCT | GGTGTTTACCTGTTCACG |
| 5022 | 4A6 | TCCGTGTCCAGCGCT | TCGGCATCCAGCCTCTACTCT | GGTGTTTACCTGTTCACG |
| 5014 | 4A7 | TCCGTGTCCAGCGCT | TCGGCATCCAGCCTCTACTCT | GGTGTTTACCTGTTCACG |
| 5023 | 4A8 | TCCGTGTCCAGCGCT | TCGGCATCCAGCCTCTACTCT | GGTGTTTACCTGTTCACG |
| 6494 | 4A9 | TCCGTGTCCAGCGCT | TCGGCATCCAGCCTCTACTCT | GGTGTTTACCTGTTCACG |
| 5025 | 4A10 | TCCGTGTCCAGCGCT | TCGGCATCCAGCCTCTACTCT | GGTGTTTACCTGTTCACG |
| 6495 | 4A11 | TCCGTGTCCAGCGCT | TCGGCATCCAGCCTCTACTCT | TCTTCTTATTCTCTGATCACG |
| 6496 | 4A12 | TCCGTGTCCAGCGCT | TCGGCATCCAGCCTCTACTCT | TCTTCTTATTCTCTGATCACG |
| 6497 | 4B1 | TCCGTGTCCAGCGCT | TCGGCATCCAGCCTCTACTCT | TCTTCTTATTCTCTGATCACG |
| 5027 | 4B2 | TCCGTGTCCAGCGCT | TCGGCATCCAGCCTCTACTCT | TCTTCTTATTCTCTGATCACG |
| 5028 | 4B3 | TCCGTGTCCAGCGCT | TCGGCATCCAGCCTCTACTCT | TCTTCTTATTCTCTGATCACG |
| 5029 | 4B4 | TCCGTGTCCAGCGCT | TCGGCATCCAGCCTCTACTCT | TCTTCTTATTCTCTGATCACG |
| 5030 | 4B5 | TCCGTGTCCAGCGCT | TCGGCATCCAGCCTCTACTCT | TCTTCTTATTCTCTGATCACG |
| 5031 | 4B6 | TCCGTGTCCAGCGCT | TCGGCATCCAGCCTCTACTCT | TCTTCTTATTCTCTGATCACG |
| 6498 | 4B7 | TCCGTGTCCAGCGCT | TCGGCATCCAGCCTCTACTCT | TCTTCTTATTCTCTGATCACG |
| 6499 | 4B8 | TCCGTGTCCAGCGCT | TCGGCATCCAGCCTCTACTCT | TCTTCTTATTCTCTGATCACG |
| 5034 | 4B9 | TCCGTGTCCAGCGCT | TCGGCATCCAGCCTCTACTCT | TGGGCTTACGGTCCGTTCACG |
| 5035 | 4B10 | TCCGTGTCCAGCGCT | TCGGCATCCAGCCTCTACTCT | TACTACCATCCGATCACG |
| 5036 | 4B11 | TCCGTGTCCAGCGCT | TCGGCATCCAGCCTCTACTCT | TACTACTCTCTGTTCACG |
| 5037 | 4B12 | TCCGTGTCCAGCGCT | TCGGCATCCAGCCTCTACTCT | TACTACTCTCTGTTCACG |
| 6500 | 4C1 | TCCGTGTCCAGCGCT | TCGGCATCCAGCCTCTACTCT | TACTACTCTCTGTTCACG |

TABLE 1c
CDR Nucleic Acid sequences for FZD4 antibodies

| Clone | | | | |
|---|---|---|---|---|
| ID | Well ID | CDR H1 | CDR H2 | CDR H3 |
| 5016 | | ATCCATTCTTCTTCTATA | GCTACTTATTCTTCTTTTGGCTC | TACCATCACCCGTTCGGTT |
| | | | TATTACT | ATGCTTTG |
| 5017 | 4A1 | CTCTATTATACTGATATG | TCTATTTCTCTTTTTTTTGGCTAT | TACTTGGCTATG |
| | | | GTTTCT | |
| 5018 | 4A2 | ATCTATTTTTCTTCTATC | TCTAATTATCCTTCTTTTGGCTC | TACCATTTCCCGTTCGCTT |
| | | | TAATTCT | ATTCTTTG |
| 5019 | 4A3 | ATCGGTTCTTCTTCTATC | TCTATTTATTCTGCTTTTGCCTC | TACCATTTCCCGTTCGGTT |
| | | | TACTTCT | TTGCTTTG |
| 5020 | 4A4 | GTCAATTCTTCTTCTATC | GCTTTTTATTCTTCTTTTGGCGC | TACCATTTCCCGTTCGGTC |
| | | | TACTTCT | ATGCTCTG |
| 5021 | 4A5 | ATCGGTTCTTCTTCTATC | GCTTATTATTCTGCTTTTGCCTC | TACCATTACCCGTTCGGTC |
| | | | TAGTTCT | ATGCTTTG |
| 5022 | 4A6 | ATCCATTTTTCTTCTATC | TGTAGTTATCCTTCTTTTGGCTC | TACCATTACCCGTTCGGTC |
| | | | TACTTCT | ATGCTCTG |
| 5014 | 4A7 | ATCTATTCTGCTTCTATC | TCTCGTTATCCTTCTTTTGGCTC | TACCATTACCCGTTCGGTA |
| | | | TACTTCT | CTGCTTTG |
| 5023 | 4A8 | ATCCATTCTTCTTCTATC | GCTATTTATTCTTCTTTTAGCGC | TACCATTACCCGTTCGGTT |
| | | | TAATTCT | ATGCTTTG |
| 6494 | 4A9 | ATCTATTTTTCTTCTATC | TCTAATTATCCTGCTTTTGGCTC | TACCATTACCCGTTCGGTT |
| | | | TACTTCT | ATGCTATG |
| 5025 | 4A10 | ATCTATTCTTCTTCTATC | TCTATTTATTCTGCTTTTCTCTCT | TACCATTACCCGCACGGT |
| | | | ACTACT | CATGCTTTG |
| 6495 | 4A11 | CTCTCTTATTCTTTTTTC | TCTATTTATCCTTCTTCTGGCTA | CCGGCTCCNTTTTCTTACC |
| | | | TACTTAT | ATGTTCTG |
| 6496 | 4A12 | CTCTCTTTTTATTTTTTG | TCTATTTATCCTTATTCTGGCTA | GCGGCTCCGGGTTCTTAC |
| | | | TACTTAT | CATCCTATG |
| 6497 | 4B1 | CTTTCTTCTTATTATATG | TCTATTTATCCTTTTCATGCCTC | GCGGCTCCCTATTTTTACG |
| | | | TACTTAT | GTGTTATG |
| 5027 | 4B2 | TCCTCTTTTTATTTTATG | ACTGTTTATCCTTATCTTGACTA | GCGTTTCCGGGTTCTTAC |
| | | | TACTTAT | CATCCTATG |
| 5028 | 4B3 | CTCTCTTATTATTATATG | TCTATTTATCCTTATTCTCGCAA | CGCGTATCCGTTTTCTTAC |
| | | | TACTTTT | CATTTTATG |
| 5029 | 4B4 | CTCTCTTATTATTATATG | TCTATTTATCCTTTTTCTGGCTAT | CCGTCTGCGTTTTCTTACC |
| | | | TCTACT | ATCCTATG |
| 5030 | 4B5 | CTCAGTTTTTATTATATG | TCTATTTATCCTTATTATGCCTAT | CCGGTTGCGGGTGCTTAC |
| | | | ACTTAT | CATCCTATG |
| 5031 | 4B6 | ATCGCTTCTTATTTTACG | TCTATTTATCTTTCTTTTGGCTAT | TCGTCTCTGGGTTTTTACA |
| | | | GGTTAT | ATGGTATG |
| 6498 | 4B7 | TTTTCTTCTTCTTCTATA | TGTTGTAATTCTGCTTATCGCTA | ACTGTTCGTGGATCCAAAA |
| | | | TGGTCCT | AACCGTACTTCTCTGGTTG |
| | | | | GGCTATG |
| 6499 | 4B8 | CTCTCTTATTATTTTATG | TCTATTTATCCTTATGCTGGCAA | ACGTATCCGGGTTATTACT |
| | | | TACTTAT | ATATTCTG |
| 5034 | 4B9 | ATCTATTATTATCCTATG | TCTTTTTATTCTTATTATAGCTTT | TCTGGTGTGGGTGGTGAT |
| | | | ACTTAT | CACGCTTTG |
| 5035 | 4B10 | TTCTCTGCTTATAATATC | TCTCTTTATACTTCTTATGGCTA | GTTTGGTACGTTGTTCAG |
| | | | TACTTAT | |
| 5036 | 4B11 | TTTTCTTCTTCTTCTATA | TATATTTATCCTTTTAATGGCTAT | GGTTACTTCTACACTTGGG |
| | | | AGTTAT | GTGGTATG |
| 5037 | 4B12 | TTTTCTTCTTCTTCTATA | TATATTTATCCTTCTTATGACTAT | GGTTACTTCTACACTTGGG |
| | | | ACTTAT | GTGGTATG |
| 6500 | 4C1 | ATCTATTATTTTGGTATG | TATATTTCTCCTCCTTATGGCTT | GGTTACTACTACTCTTGGG |
| | | | TACTTAT | GTGGTATG |

Also described herein are heavy chain and light chain variable regions. Table 2 provides exemplary variable domain sequences for the Fab heavy and light chains, from clone 5017. Antibodies comprising the sequences in Table 2 or sequences substantially identical thereto, wherein the CDRs are a CDR sequence set identified in Tables 1a or 3a are also contemplated. In another embodiment, the antibody comprises a heavy chain variable region comprising: i) a heavy chain amino acid sequence as set forth in Table 2; ii) an amino acid sequence with at least 50%, at least 60%, at least 70%, at least 80%, at least 90%, at least 95%, at least 98% or at least 99% sequence identity to the heavy chain amino acid sequence as set forth in Table 2, wherein the CDR sequences are a CDR sequence set as set forth in Table 1a or 3a, or iii) a conservatively substituted amino acid sequence of i) wherein the CDR sequences are a CDR sequence set as set forth in Table 1a or 3a.

In another embodiment, the antibody comprises a light chain variable region comprising i) a light chain amino acid sequence as set forth in Table 2, ii) an amino acid sequence with at least 50%, at least 60%, at least 70%, at least 80%, at least 90%, at least 95%, at least 98% or at least 99% sequence identity to the light chain amino acid sequence as set forth in Table 2, wherein the CDR sequences are a CDR sequence set as set forth in Table 1a or 3a, or iii) a conservatively substituted amino acid sequence of i) wherein the CDR sequences are a CDR sequence set as set forth in Table 1a or 3a.

TABLE 2
Eample of full-length sequences
for FZD4 antibody 5017

Light chain (hK) amino acid sequence:
DIQMTQSPSSLSASVGDRVTITCRASQSVSSAVAWYQQKPGKAPKLLIY
SASSLYSGVPSRFSGSRSGTDFTLTISSLQPEDFATYYCQQAAYHWPPL
FTFGQGTKVEIKRTVAAPSVFIFPPSDEQLKSGTASVVCLLNNFYPREA
KVQWKVDNALQSGNSQESVTEQDSKDSTYSLSSTLTLSKADYEKHKVYA
CEVTHQGLSSPVTKSFNRGEC
Light chain (hK) nucleic acid sequence:
GATATCCAGATGACCCAGTCCCCGAGCTCCCTGTCCGCCTCTGTGGGCG
ATAGGGTCACCATCACCTGCCGTGCCAGTCAGTCCGTGTCCAGCGCTGT
AGCCTGGTATCAACAGAAACCAGGAAAAGCTCCGAAGCTTCTGATTTAC
TCGGCATCCAGCCTCTACTCTACTCTGGAGTCCCTTCTCGCTTCTCTGG
TAGCCGTTCCGGGACGGATTTCACTCTGACCATCAGCAGTCTGCAGCCG
GAAGACTTCGCAACTTATTACTGTCAGCAAGCTGCTTACCATTGGCCGC
CGCTGTTCACGACGTTCGGACAGGGTACCAAGGTGGAGATCAAACGTAC
GGTGGCTGCACCATCTGTCTTCATCTTCCCGCCATCTGATGAGCAGTTG
AAATCTGGAACTGCCTCTGTTGTGTGCCTGCTGAATAACTTCTATCCCA
GAGAGGCCAAAGTACAGTGGAAGGTGGATAACGCCCTCCAATCGGGTAA
CTCCCAGGAGAGTGTCACAGAGCAGGACAGCAAGGACAGCACCTACAGC
CTCAGCAGCACCCTGACGCTGAGCAAAGCAGACTACGAGAAACACAAAG
TCTACGCCTGCGAAGTCACCCATCAGGGCCTGAGCTCGCCCGTCACAAA
GAGCTTCAACAGGGGAGAGTGT
Heavy chain (hG1) amino acid sequence:
EVQLVESGGGLVQPGGSLRLSCAASGFNLYYTDMHWVRQAPGKGLEWVA
SISLFFGYVSYADSVKGRFTISADTSKNTAYLQMNSLRAEDTAVYYCAR
YLAMDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSL
SPGK
Heavy chain (hG1) nucleic acid sequence:
GAGGTTCAGCTGGTGGAGTCTGGCGGTGGCCTGGTGCAGCCAGGGGGCT
CACTCCGTTTGTCCTGTGCAGCTTCTGGCTTCAACCTCTATTATACTGA
TATGCACTGGGTGCGTCAGGCCCCGGGTAAGGGCCTGGAATGGGTTGCA
TCTATTTCTCTTTTTTTTGGCTATGTTTCTTATGCCGATAGCGTCAAGG
GCCGTTTCACTATAAGCGCAGACACATCCAAAAACACAGCCTACCTACA
AATGAACAGCTTAAGAGCTGAGGACACTGCCGTCTATTATTGTGCTCGC
TACTTGGCTATGGACTACTGGGGTCAAGGAACCCTGGTCACCGTCTCCT
CGGCTAGCACCAAGGGCCCATCGGTCTTCCCCCTGGCACCCTCCTCCAA
GAGCACCTCTGGGGGCACAGCGGCCCTGGGCTGCCTGGTCAAGGACTAC
TTCCCCGAACCGGTGACGGTGTCGTGGAACTCAGGCGCCCTGACCAGCG
GCGTGCACACCTTCCCGGCTGTCCTACAGTCCTCAGGACTCTACTCCCT
CAGCAGCGTGGTGACCGTGCCTCCAGCAGCTTGGGCACCCAGACCTACC
ATCTGCAACGTGAATCACAAGCCCAGCAACACCAAGGTGGACAAGAAAG
TTGAGCCCAAATCTTGTGACAAAACTCACACATGCCCACCGTGCCCAGC
ACCTGAACTCCTGGGGGGACCGTCAGTCTTCCTCTTCCCCCCAAAACCC
AAGGACACCCTCATGATCTCCCGGACCCCTGAGGTCACATGCGTGGTGG
TGGACGTGAGCCACGAAGACCCTGAGGTCAAGTTCAACTGGTACGTGGA
CGGCGTGGAGGTGCATAATGCCAAGACAAAGCCGCGGGAGGAGCAGTAC
AACAGCACGTACCGTGTGGTCAGCGTCCTCACCGTCCTGCACCAGGACT
GGCTGAATGGCAAGGAGTACAAGTGCAAGGTCTCCAACAAAGCCCTCCC
AGCCCCCATCGAGAAAACCATCTCCAAAGCCAAAGGGCAGCCCCGAGAA
CCACAGGTGTACACCCTGCCCCCATCCCGGGAGGAGATGACCAAGAACC
AGGTCAGCCTGACCTGCCTGGTCAAAGGCTTCTATCCCAGCGACATCGC
CGTGGAGTGGGAGAGCAATGGGCAGCCGGAGAACAACTACAAGACCACG
CCTCCCGTGCTGGACTCCGACGGCTCCTTCTTCCTCTACAGCAAGCTCA
CCGTGGACAAGAGCAGGTGGCAGCAGGGGAACGTCTTCTCATGCTCCGT
GATGCATGAGGCTCTGCACAACCACTACACGCAGAAGAGCCTCTCCCTG
TCTCCGGGTAAA
Underlined identifes CDR sequence
Bold identifies CDR variation from the library (Antibody
clone ID 5017 shown as example here)
Italics represent constant domain sequence

In another embodiment, the antibody comprises a CDR sequence set selected from the CDR sequence sets in Table 3a, that is, for clones 5038 to 5081.

Table 3a—CDR Amino Acid sequences for FZD4 antibodies

TABLE 3a
CDR Amino Acid sequences for FZD4 antibodies

| Clone | Well | CDR | CDR | CDR | CDR | CDR | CDR |
|---|---|---|---|---|---|---|---|
| ID | ID | L1 | L2 | L3 | H1 | H2 | H3 |
| 5038 | A01 | SVSSA | SASS | HPWS | ISYY | SIYS | SSFS |
| | | | LYS | GGYL | YM | YYGY | WAM |
| | | | | I | | TY | |
| 5039 | A02 | SVSSA | SASS | PVGY | IYSY | SIYS | SSFY |
| | | | LYS | WGVP | YM | SSSS | WAL |
| | | | | I | | TY | |
| 5040 | A03 | SVSSA | SASS | VSGG | LSYY | SIYP | SWFG |
| | | | LYS | AHAL | YM | SSSY | WGI |
| | | | | I | | TY | |
| 5041 | A04 | SVSSA | SASS | VSSA | ISYY | SIYS | YWFS |
| | | | LYS | YPI | YM | SSSY | YGYA |
| | | | | | | TS | SYPA |
| | | | | | | | F |
| 5042 | A05 | SVSSA | SASS | FWGV | IYYY | YISS | HPWY |
| | | | LYS | PI | SI | YSGS | GM |
| | | | | | | TY | |
| 5043 | A06 | SVSSA | SASS | SYYH | LYSY | SIYS | SAFY |
| | | | LYS | YAAL | YM | SYGY | WAL |
| | | | | I | | TY | |
| 5044 | A07 | SVSSA | SASS | WYYA | LSSY | YISS | PAPG |
| | | | LYS | PI | SM | YYGY | HWGF |
| | | | | | | TY | |
| 5045 | A08 | SVSSA | SASS | SHSY | ISYY | SIYP | SSFF |
| | | | LYS | SLI | YM | SSSS | WAM |
| | | | | | | TY | |
| 5046 | A09 | SVSSA | SASS | SGYG | ISYY | SIYS | SAFY |
| | | | LYS | PF | YI | SSGY | WAM |
| | | | | | | TY | |
| 5047 | A10 | SVSSA | SASS | SWSS | LSYS | YISS | HFFA |
| | | | LYS | PI | SM | YSGS | M |
| | | | | | | TS | |
| 5048 | A11 | SVSSA | SASS | HYSV | ISYY | SISS | SWWA |
| | | | LYS | YASL | YM | YYGS | WAF |
| | | | | I | | TY | |
| 5049 | A12 | SVSSA | SASS | PHPP | IYYY | SIYS | safg |
| | | | LYS | SL! | YM | YYGS | wal |
| | | | | | | TY | |
| 5050 | B01 | SVSSA | SASS | VAYS | LSYY | SIYP | SSFF |
| | | | LYS | HVGL | YM | YSGY | FAM |
| | | | | I | | TY | |
| 5051 | 802 | SVSSA | SASS | GYGA | LYYY | YISS | PYYW |
| | | | LYS | PI | SI | YSGS | SGGF |
| | | | | | | TY | |
| 5052 | 803 | SVSSA | SASS | SWYS | ISSY | YISP | HPSS |
| | | | LYS | LI | YI | YYGY | SWFS |
| | | | | | | TS | FGAL |
| 5053 | 804 | SVSSA | SASS | PGYL | LYSY | SIYS | SAFY |
| | | | LYS | F | YM | SSGY | WAM |
| | | | | | | TY | |
| 5054 | B05 | SVSSA | SASS | VWFG | LSYY | SISS | SAFY |
| | | | LYS | LI | YM | SSGY | WAF |
| | | | | | | TY | |
| 5055 | B06 | SVSSA | SASS | VYYG | LYSY | SIYS | SSYA |
| | | | LYS | SPLF | YM | YSSS | WAM |
| | | | | | | TY | |
| 5056 | B07 | SVSSA | SASS | HAHS | LSYY | SISP | SSFY |
| | | | LYS | PLI | YM | SSSY | WAI |
| | | | | | | TY | |
| 5057 | B0S | SVSSA | SASS | SSAY | FSSS | SIYS | SPWG |
| | | | LYS | YPF | SI | YYGY | SGWA |
| | | | | | | TY | GF |
| 5058 | B09 | SVSSA | SASS | GHAS | LSYY | YISP | PAVW |
| | | | LYS | PI | SI | YYGY | VGL |
| | | | | | | TY | |
| 5059 | B10 | SVSSA | SASS | SSGG | LYSY | SISP | SWVF |
| | | | LYS | WSLI | YI | YSSS | WAL |
| | | | | | | TY | |
| 5060 | B11 | SVSSA | SASS | VAWS | LSYY | SIYS | SWYW |
| | | | LYS | SFLI | YM | SYGS | GM |
| | | | | | | TY | |
| 5061 | B12 | SVSSA | SASS | SVAA | LSSY | SIYS | SWVY |
| | | | LYS | ASLI | YM | SYGS | WAL |
| | | | | | | TY | |
| 5062 | C01 | SVSSA | SASS | SGWW | ISYY | SIYS | SSYA |
| | | | LYS | GVSL | YI | SSSY | WAI |
| | | | | I | | TY | |
| 5063 | C02 | SVSSA | SASS | SYAA | ISYY | SIYP | SSFY |
| | | | LYS | YLF | YI | SSGY | WAM |
| | | | | | | TY | |
| 5064 | C03 | SVSSA | SASS | HGSL | LSYY | SIYP | HGAS |
| | | | LYS | F | YI | YSGS | FGSG |
| | | | | | | TY | APAF |
| 5065 | C04 | SVSSA | SASS | YAGV | IYSY | SIYS | SCFF |
| | | | LYS | SNLF | YM | YYGS | WAM |
| | | | | | | TY | |
| 5066 | C05 | SVSSA | SASS | GWPY | ISSY | SIYP | WAFF |
| | | | LYS | SALF | YM | SYGS | GL |
| | | | | | | TY | |
| 5067 | C06 | SVSSA | SASS | SGYY | LSYY | YISS | SSFY |
| | | | LYS | PSLF | SM | YSSY | FAM |
| | | | | | | TY | |
| 5068 | C07 | SVSSA | SASS | SYHS | ISSY | SIYS | SAFS |
| | | | LYS | GSGL | YM | YYSS | WAI |
| | | | | I | | TY | |
| 5069 | C08 | SVSSA | SASS | HGYS | ISYY | SISP | SGFY |
| | | | LYS | ASLI | YM | YSSS | WAL |
| | | | | | | TY | |
| 5070 | C09 | SVSSA | SASS | SGYY | LYSY | YISS | PSVG |
| | | | LYS | PSLF | SI | SYGY | YAAF |
| | | | | | | TS | |
| 5071 | C10 | SVSSA | SASS | APGW | LSYY | SISP | SWVG |
| | | | LYS | ALF | YM | YSSY | WGL |
| | | | | | | TY | |
| 5072 | C11 | SVSSA | SASS | GHSS | LYSY | YISP | SSVG |
| | | | LYS | PI | SI | YSGY | YVAM |
| | | | | | | TS | |
| 5073 | C12 | SVSSA | SASS | GWPS | ISYY | SIYP | SWYW |
| | | | LYS | LF | YI | YYSY | AF |
| | | | | | | TY | |
| 5074 | D01 | SVSSA | SASS | VPGY | ISSY | SISP | YYYS |
| | | | LYS | PVPI | YM | YYGY | SSVY |
| | | | | | | TS | FWYA |
| | | | | | | | AL |
| 5075 | D02 | SVSSA | SASS | HYYS | LYYY | SISP | SSFF |
| | | | LYS | HLI | YI | SYSS | WAL |
| | | | | | | TY | |
| 5076 | D03 | SVSSA | SASS | GPAS | IYSY | SISS | SWVY |
| | | | LYS | SLI | YI | SYSS | WAI |
| | | | | | | TY | |
| 5077 | D04 | SVSSA | SASS | SVGS | ISYY | SISP | SWVG |
| | | | LYS | SYYL | YM | YSSY | WGI |
| | | | | I | | TY | |
| 5078 | D05 | SVSSA | SASS | YYGP | ISYY | SISP | SSVY |
| | | | LYS | WVLI | YM | YSSS | WAL |
| | | | | | | TY | |
| 5079 | D06 | SVSSA | SASS | AASW | ISYS | SIYP | WGGW |
| | | | LYS | GYPF | YI | YSGS | GSGG |
| | | | | | | TS | YFYA |
| | | | | | | | AL |
| 5080 | D07 | SVSSA | SASS | HWSY | ISYY | SISS | FWYP |
| | | | LYS | P1 | SM | YYSS | GM |
| | | | | | | TS | |
| 5081 | D08 | SVSSA | SASS | GGWG | LSYY | SIYS | SSFA |
| | | | LYS | PF | YM | YSGY | WAF |
| | | | | | | TY | |

Table 3b—CDR Light Chain Nucleic Acid sequences for FZD4 antibodies

TABLE 3b
CDR Nucleic Acid sequences for
FZD4 antibodies

| Clone | Wed | CDR | CDR | CDR |
|---|---|---|---|---|
| ID | ID | L1 | L2 | L3 |
| 5038 | A01 | TCCGT | TCGGC | CATCC |
| | | GTCCA | ATCCA | GTGGT |
| | | GCGCT | GCCTC | CTGGT |
| | | | TACTC | GGTTA |
| | | | T | CCTGA |
| | | | | TC |
| 5039 | A02 | TCCGT | TCGGC | CCGGT |
| | | GTCCA | ATCCA | TGGTT |
| | | GCGCT | GCCTC | ACTGG |
| | | | TACTC | GGTGT |
| | | | T | TCCGA |
| | | | | TC |
| 5040 | A03 | TCCGT | TCGGC | GTTTC |
| | | GTCCA | ATCCA | TGGTG |
| | | GCGCT | GCCTC | GTGCT |
| | | | TACTC | CATGC |
| | | | T | TCTGA |
| | | | | TC |
| 5041 | A04 | TCCGT | TCGGC | GTTTC |
| | | GTCCA | ATCCA | TTCTG |
| | | GCGCT | GCCTC | CTTAC |
| | | | TACTC | CCGAT |
| | | | T | C |
| 5042 | A05 | TCCGT | TCGGC | TTCTG |
| | | GTCCA | ATCCA | GGGTG |
| | | GCGCT | GCCTC | TTCCG |
| | | | TACTC | ATC |
| | | | T | |
| 5043 | A06 | TCCGT | TCGGC | TCTTA |
| | | GTCCA | ATCCA | CTACC |
| | | GCGCT | GCCTC | ATTAC |
| | | | TACTC | GCTGC |
| | | | T | TCTGA |
| | | | | TC |
| 5044 | A07 | TCCGT | TCGGC | TGGTA |
| | | GTCCA | ATCCA | CTACG |
| | | GCGCT | GCCTC | CTCCG |
| | | | TACTC | ATC |
| | | | T | |
| 5045 | A08 | TCCGT | TCGGC | TCTCA |
| | | GTCCA | ATCCA | TTCTT |
| | | GCGCT | GCCTC | ACTCT |
| | | | TACTC | CTGAT |
| | | | T | C |
| 5046 | A09 | TCCGT | TCGGC | TCTGG |
| | | GTCCA | ATCCA | TTACG |
| | | GCGCT | GCCTC | GTCCG |
| | | | TACTC | TTC |
| | | | T | |
| 5047 | A10 | TCCGT | TCGGC | TCTTG |
| | | GTCCA | ATCCA | GTCTT |
| | | GCGCT | GCCTC | CTCCG |
| | | | TACTC | ATC |
| | | | T | |
| 5048 | A11 | TCCGT | TCGGC | CATTA |
| | | GTCCA | ATCCA | CTCTG |
| | | GCGCT | GCCTC | TTTAC |
| | | | TACTC | GCTTC |
| | | | T | TCT |
| 5049 | A12 | TCCGT | TCGGC | GATCC |
| | | GTCCA | ATCCA | CGCAT |
| | | GCGCT | GCCTC | CCGCC |
| | | | TACTC | GTCTC |
| | | | T | TGATC |
| 5050 | B01 | TCCGT | TCGGC | GTTGC |
| | | GTCCA | ATCCA | CTACT |
| | | GCGCT | GCCTC | CTCAT |
| | | | TACTC | GTTGG |
| | | | T | TC |
| 5051 | B02 | TCCGT | TCGGC | TGATC |
| | | GTCCA | ATCCA | GGTTA |
| | | GCGCT | GCCTC | CGGTG |
| | | | TACTC | CTCCG |
| | | | T | ATC |
| 5052 | B03 | TCCGT | TCGGC | TCTTG |
| | | GTCCA | ATCCA | GTACT |
| | | GCGCT | GCCTC | CTCTG |
| | | | TACTC | ATC |
| | | | T | |
| 5053 | B04 | TCCGT | TCGGC | CCGGG |
| | | GTCCA | ATCCA | TTACC |
| | | GCGCT | GCCTC | TGTTC |
| | | | TACTC | |
| | | | T | |
| 5054 | B05 | TCCGT | TCGGC | GTTTG |
| | | GTCCA | ATCCA | GTTCG |
| | | GCGCT | GCCTC | GTCTG |
| | | | TACTC | ATC |
| | | | T | |
| 5055 | B05 | TCCGT | TCGGC | GTTTA |
| | | GTCCA | ATCCA | CTACG |
| | | GCGCT | GCCTC | GTTCT |
| | | | TACTC | CCGCT |
| | | | T | GTTC |
| 5056 | B07 | TCCGT | TCGGC | CATGC |
| | | GTCCA | ATCCA | TCATT |
| | | GCGCT | GCCTC | CTCCG |
| | | | TACTC | CTGAT |
| | | | T | C |
| 5057 | B08 | TCCGT | TCGGC | TCTTC |
| | | GTCCA | ATCCA | TGCTT |
| | | GCGCT | GCCTC | ACTAC |
| | | | TACTC | CCGTT |
| | | | T | C |
| 5058 | B09 | TCCGT | TCGGC | GGTCA |
| | | GTCCA | ATCCA | TGCTT |
| | | GCGCT | GCCTC | CTCCG |
| | | | TACTC | ATC |
| | | | T | |
| 5059 | B10 | TCCGT | TCGGC | TCTTC |
| | | GTCCA | ATCCA | TGGTG |
| | | GCGCT | GCCTC | GTTGG |
| | | | TACTC | TCTCT |
| | | | T | GATC |
| 5060 | B11 | TCCGT | TCGGC | GTTGC |
| | | GTCCA | ATCCA | TTGGT |
| | | GCGCT | GCCTC | CTTCT |
| | | | TACTC | TTCCT |
| | | | T | GATC |
| 5061 | B12 | TCCGT | TCGGC | TCTGT |
| | | GTCCA | ATCCA | TGCTG |
| | | GCGCT | GCCTC | CTGCT |
| | | | TACTC | TCTCT |
| | | | T | GATC |
| 5062 | C01 | TCCGT | TCGGC | TCTGG |
| | | GTCCA | ATCCA | TTGGT |
| | | GCGCT | GCCTC | GGGGT |
| | | | TACTC | GTTTC |
| | | | T | TCTGA |
| | | | | TC |
| 5063 | C02 | TCCGT | TCGGC | TCTTA |
| | | GTCCA | ATCCA | CGCTG |
| | | GCGCT | GCCTC | CTTAC |
| | | | TACTC | CTGTT |
| | | | T | C |
| 5064 | C03 | TCCGT | TCGGC | CATGG |
| | | GTCCA | ATCCA | TTCTC |
| | | GCGCT | GCCTC | TGTTC |
| | | | TACTC | |
| | | | T | |
| 5065 | C04 | TCCGT | TCGGC | *** |
| | | GTCCA | ATCCA | |
| | | GCGCT | GCCTC | |
| | | | TACTC | |
| | | | T | |
| 5066 | C05 | TCCGT | TCGGC | GGTTG |
| | | GTCCA | ATCCA | GCCGT |
| | | GCGCT | GCCTC | ACTCT |
| | | | TACTC | GCTCT |
| | | | T | GTTC |
| 5067 | C06 | TCCGT | TCGGC | TCTGG |
| | | GTCCA | ATCCA | TTACT |
| | | GCGCT | GCCTC | ACCCG |
| | | | TACTC | TCTCT |
| | | | T | GTTC |
| 5068 | C07 | TCCGT | TCGGC | TCTTA |
| | | GTCCA | ATCCA | CCATT |
| | | GCGCT | GCCTC | CTGGT |
| | | | TACTC | TCTGG |
| | | | T | TCTGA |
| | | | | TC |
| 5069 | C08 | TCCGT | TCGGC | CATGG |
| | | GTCCA | ATCCA | TTACT |
| | | GCGCT | GCCTC | CTGCT |
| | | | TACTC | TCTCT |
| | | | T | GATC |
| 5070 | C09 | TCCGT | TCGGC | TCTGG |
| | | GTCCA | ATCCA | TTACT |
| | | GCGCT | GCCTC | ACCCG |
| | | | TACTC | TCTCT |
| | | | T | GTTC |
| 5071 | C10 | TCCGT | TCGGC | GCTCC |
| | | GTCCA | ATCCA | GGGTT |
| | | GCGCT | GCCTC | GGGCT |
| | | | TACTC | CTGTT |
| | | | T | C |
| 5072 | C11 | TCCGT | TCGGC | GGTCA |
| | | GTCCA | ATCCA | TTCTT |
| | | GCGCT | GCCTC | CTCCG |
| | | | TACTC | ATC |
| | | | T | |
| 5073 | C12 | TCCGT | TCGGC | GGTTG |
| | | GTCCA | ATCCA | GCCGT |
| | | GCGCT | GCCTC | CTCTG |
| | | | TACTC | TTC |
| | | | T | |
| 5074 | D01 | TCCGT | TCGGC | GTTCC |
| | | GTCCA | ATCCA | GGGTT |
| | | GCGCT | GCCTC | ACCCG |
| | | | TACTC | GTTCC |
| | | | T | GATC |
| 5075 | D02 | TCCGT | TCGGC | CATTA |
| | | GTCCA | ATCCA | CTACT |
| | | GCGCT | GCCTC | CTCAT |
| | | | TACTC | CTGAT |
| | | | T | C |
| 5076 | D03 | TCCGT | TCGGC | GGTCC |
| | | GTCCA | ATCCA | GGCTT |
| | | GCGCT | GCCTC | CTTCT |
| | | | TACTC | CTGAT |
| | | | T | C |
| 5077 | D04 | TCCGT | TCGGC | TCTGT |
| | | GTCCA | ATCCA | TGGTT |
| | | GCGCT | GCCTC | CTTCT |
| | | | TACTC | TACTA |
| | | | T | CCTGA |
| | | | | TC |
| 5078 | D05 | TCCGT | TCGGC | TACTA |
| | | GTCCA | ATCCA | CGGTC |
| | | GCGCT | GCCTC | CGTGG |
| | | | TACTC | GTTCT |
| | | | T | GATC |
| 5079 | D06 | TCCGT | TCGGC | GCTGC |
| | | GTCCA | ATCCA | TTCTT |
| | | GCGCT | GCCTC | GGGGT |
| | | | TACTC | TACCC |
| | | | T | GTTC |
| 5080 | D07 | TCCGT | TCGGC | CATTG |
| | | GTCCA | ATCCA | GTCTT |
| | | GCGCT | GCCTC | ACCCG |
| | | | TACTC | ATC |
| | | | T | |
| 5081 | D08 | TCCGT | TCGGC | GGTGG |
| | | GTCCA | ATCCA | TTGGG |
| | | GCGCT | GCCTC | GTCCG |
| | | | TACTC | TTC |
| | | | T | |

In some embodiments, the variable domain sequences are at least 95%, 96%, 97%, 98%, or 99% similar outside of the CDR regions and the CDR sequence set is 100% identical to the amino acid sequences provided in Table 1a or 3a.

Table 3c—CDR Heavy Chain Nucleic Acid sequences for FZD4 antibodies

TABLE 3c
CDR Nucleic Acid sequences for FZD4 antibodies

| Clone | Well | | | |
|---|---|---|---|---|
| ID | ID | CDR H1 | CDR H2 | CDRH3 |
| 5038 | A01 | TCTATTTATTC | TCTATTTATTCTTAT | TCTTCTTTCTC |
| | | TTATTATG | TATGGCTATACTTAT | TTGGGCTA |
| | | GCTATACTTAT | | TG |
| 5039 | A02 | TCTATTTATTC | TCTATTTATTCTTCT | TCTTCTTTCTA |
| | | TTCTTCTA | TCTAGCTCTACTTAT | CTGGGCTT |
| | | GCTCTACTTAT | | TG |
| 5040 | A03 | TCTATTTATCC | TCTATTTATCCTTCT | TCTTGGTTCGG |
| | | TTCTTCTA | TCTAGCTATACTTAT | TTGGGGT |
| | | GCTATACTTAT | | ATT |
| 5041 | A04 | TCTATTTATTC | TCTATTTATTCTTCT | TACTGGTTCTC |
| | | TTCTTCTA | TCTAGCTATACTTCT | TTACGGTT |
| | | GCTATACTTCT | | ACGCTTCTTAC |
| | | | | CCGGCTTT |
| 5042 | A05 | TATATTTCTTC | TATATTTCTTCTTAT | CATCCGTGGTA |
| | | TTATTCTG | TCTGGCTCTACTTAT | CGGTATG |
| | | GCTCTACTTAT | | |
| 5043 | A06 | CTCTATTCTTA | TCTATTTATTCTTCT | TCTGCTTTCTA |
| | | TTATATG | TATGGCTATACTTAT | CTGGGCTT |
| 5044 | A07 | TATATTTCTTC | TATATTTCTTCTTAT | ToCCGGCTCCG |
| | | TTATTATG | TATGGCTATACTTAT | GGTCATTGG |
| | | GCTATACTTAT | | GGTTTT |
| 5045 | A08 | TCTATTTATCC | TCTATTTATCCTTCT | TCTTCTTTCTT |
| | | TTCTTCTA | TCTAGCTCTACTTAT | CTGGGCTA |
| | | GCTCTACTTAT | | TG |
| 5046 | A09 | TCTATTTATTC | TCTATTTATTCTTCT | TCTGCTTTCTA |
| | | TTCTTCTG | TCTGGCTATACTTAT | CTGGGCTA |
| | | GCTATACTTAT | | TG |
| 5047 | A10 | CTCTCTTATTC | TATATTTCTTCTTAT | CATTTCTTCGC |
| | | TTCTATG | TCTGGCTCTACTTCT | TATG |
| 5048 | A11 | TCTATTTCTTC | TCTATTTCTTCTTAT | TCTTGGTGGGC |
| | | TTATTATG | TATGGCTCTACTTAT | TTGGGCT |
| | | GCTCTACTTAT | | TTT |
| 5049 | A12 | ATCTATTATTA | TCTATTTATTCTTAT | TCTGCTTTCGG |
| | | TTATATG | TATGGCTCTACTTAT | TTGGGCTT |
| 5050 | B01 | TCTATTTATCC | TCTATTTATCCTTAT | TGTCTTCTTTC |
| | | TTATTCTG | TCTGGCTATACTTAT | TTCTTCGCTAT |
| | | GCTATACTTAT | | G |
| 5051 | B02 | TATATTTCTTC | TATATTTCTTCTTAT | CCGTACTACTG |
| | | TTATTCTG | TCTGGCTCTACTTAT | GTCTGGT |
| | | GCTCTACTTAT | | GGTTTT |
| 5052 | B03 | TATATTTCTCC | TATATTTCTCCTTAT | CATCCGTCTTC |
| | | TTATTATG | TATGGCTATACTTCT | TTCTTGGT |
| | | GCTATACTTCT | | TCTCTTTCGGT |
| | | | | GCTTTG |
| 5053 | B04 | TCTATTTATTC | TCTATTTATTCTTCT | TCTGCTTTCTA |
| | | TTCTTCTG | TCTGGCTATACTTAT | CTGGGCTA |
| | | GCTATACTTAT | | TG |
| 5054 | B05 | TCTATTTCTTG | TCTATTTCTTCTTCT | TCTGCTTTCTA |
| | | TTCTTCTG | TCTGGCTATACTTAT | CTGGGCTT |
| | | GCTATACTTAT | | TT |
| 5055 | BOS | TCTATTTATTC | TCTATTTATTCTTAT | TCTTCTTACGC |
| | | TTATTCTA | TCTAGCTCTACTTAT | TTGGGCTA |
| | | GCTCTACTTAT | | TG |
| 5056 | B07 | TCTATTTCTCC | TCTATTTCTCCTTCT | TCTTCTTTCTN |
| | | TTCTTCTA | TCTAGCTATACTTAT | CTGGGCTA |
| | | GCTATACTTAT | | TT |
| 5057 | B08 | TCTATTTATTC | TCTATTTATTCTTAT | TCTCCGTGGGG |
| | | TTATTATG | TATGGCTATACTTAT | TTCTGGT |
| | | GCTATACTTAT | | TGGGCTGGTTT |
| | | | | T |
| 5058 | B09 | TATATTTCTCC | TATATTTCTCCTTAT | CCAGCTGTTTG |
| | | TTATTATG | TATGGCTATACTTAT | GGTTGGT |
| | | GCTATACTTAT | | TTG |
| 5059 | B10 | TCTATTTCTCC | TCTATTTCTCCTTAT | TCTTGGGTTTT |
| | | TTATTCTA | TCTAGCTCTACTTAT | CTGGGCTT |
| | | GCTCTACTTAT | | TG |
| 5060 | B11 | TCTATTTATTC | TCTATTTATTCTTCT | TCTTGGGTTTA |
| | | TTCTTATG | TATGGCTCTACTTAT | CTGGNTAT |
| | | GCTCTACTTAT | | G |
| 5061 | B12 | TCTATTTATTC | TCTATTTATTCTTCT | TCTTGGGTTTA |
| | | TTCTTATG | TATGGCTCTACTTAT | CTGGGCTT |
| | | GCTCTACTTAT | | TG |
| 5062 | C01 | TCTATTTATTC | TCTATTTATTCTTCT | TCTTCTTACGC |
| | | TTCTTCTA | TCTAGCTATACTTAT | TTGGGCTA |
| | | GCTATACTTAT | | TT |
| 5063 | C02 | TCTATTTATCC | TCTATTTATCCTTCT | TCTTCTTTCTA |
| | | TTCTTCTG | TCTGGCTATACTTAT | CTGGGCTA |
| | | GCTATACTTAT | | TG |
| 5064 | C03 | TCTATTTATCC | TCTATTTATCCTTAT | CATGGTGCTTC |
| | | TTATTCTG | TCTGGCTCTACTTAT | TTTCGGTT |
| | | GCTCTACTTAT | | CTGGTGCTCCG |
| | | | | GCTTTT |
| 5065 | C04 | TCTATTTATTC | TCTATTTATTCTTAT | TCTTTTTTTTC |
| | | TTATTATG | TATGGCTCTACTTAT | TGGGCTA |
| | | GCTCTACTTAT | | TG |
| 5066 | C05 | TCTATTTATCC | TCTATTTATCCTTCT | TGGGCTTTCTT |
| | | TTCTTATG | TATGGCTCTACTTAT | CGGTTTG |
| | | GCTCTACTTAT | | |
| 5067 | C06 | TATATTTCTTC | TATATTTCTTCTTAT | TCTTCTTTCTA |
| | | TTATTCTA | TCTAGCTATACTTAT | CTTCGCTA |
| | | GCTATACTTAT | | TG |
| 5068 | C07 | TCTATTTATTC | TCTATTTATTCTTAT | TCTGCTTTCTC |
| | | TTATTATA | TATAGCTCTACTTAT | TTGGGCTA |
| | | GCTCTACTTAT | | TT |
| 5069 | C08 | ATCTCTTATTA | TCTATTTCTCCTTAT | TCTGGTTTCTA |
| | | TTATATG | TCTAGCTCTACTTAT | CTGGGCTTTT |
| | | | | TG |
| 5070 | C09 | TATATTTCTTC | TATATTTCTTCTTCT | CCGTCTGTTGG |
| | | TTCTTATG | TATGGCTATACTTCT | TTACGCT |
| | | GCTATACTTCT | | GCTTTT |
| 5071 | C10 | TCTATTTCTCC | TCTATTTCTCCTTAT | TCTTGGGTTGG |
| | | TTATTCTA | TCTAGCTATACTTAT | TTGGGGT |
| | | GCTATACTTAT | | TTG |
| 5072 | C11 | CTCTATTCTTA | TATATTTCTCCTTAT | TCTTCTGTTGG |
| | | TTCTATC | TCTGGCTATACTTCT | TTACGTTG |
| | | | | CTATG |
| 5073 | C12 | ATCTCTTATTA | — | TCTTGGGTTTA |
| | | TTATATC | | CTGGGCTTTT |
| 5074 | D01 | TCTATTTCTCC | TCTATTTCTCCTTAT | TACTACTACTC |
| | | TTATTATG | TATGGCTATACTTCT | TTCTTCTG |
| | | GCTATACTTCT | | TTTACTTCTGG |
| | | | | TACGCTGC |
| | | | | TTTG |
| 5075 | D02 | TNTATTTCTCC | TNTATTTCTCCTTCT | TCTTCTTTCTT |
| | | TTCTTATA | TATAGCTCTACTTAT | CTGGGCTA |
| | | GCTCTACTTAT | | TT |
| 5076 | D03 | TCTATTTCTTC | TCTATTTCTTCTTCT | TCTTGGGTTTA |
| | | TTCTTATA | TATAGCTCTACTTAT | CTGGGCTA |
| | | GCTCTACTTAT | | TT |
| 5077 | D04 | TCTATTTCTCC | TCTATTTCTCCTTAT | TCTTGGGTTGG |
| | | TTATTCTA | TCTAGCTATACTTAT | TTGGGGT |
| | | GCTATACTTAT | | ATT |
| 5078 | D05 | TCTATTTCTCC | TCTATTTCTCCTTAT | TCTTCTGTTTA |
| | | TTATTCTA | TCTAGCTCTACTTAT | CTGGGCTT |
| | | GCTCTACTTAT | | TG |
| 5079 | D06 | TCTATTTATCC | TCTATTTATCCTTAT | TGGGGTGGTTG |
| | | TTATTCTG | TCTGGCTCTACTTCT | GGGTTCT |
| | | GCTCTACTTCT | | GGTGGTTACTT |
| | | | | CTACGCT |
| | | | | GCTTTG |
| 5080 | D07 | TCTATTTCTTC | TCTATTTCTTCTTAT | TTCTGGTACCC |
| | | TTATTATA | TATAGCTCTACTTCT | GGGTATG |
| | | GCTCTACTTCT | | |
| 5081 | D08 | TCTATTTATTC | TCTATTTATTCTTAT | TCTTCTTTCGC |
| | | TTATTCTG | TCTGGCTATACTTAT | TTGGGCTT |
| | | GCTATACTTAT | | TT |

Also provided in another embodiment, is a competing antibody that competes for binding with an antibody comprising a CDR sequence set described herein. For example, the competing antibody in one embodiment reduces binding of the antibody comprising the CDR sequence set to FZD4 CDR by at least 50%, at least 60%, at least 70%, at least 80% at least 90%, at least 95%, at least 98% or at least 99%.

A number of the antibodies described, were able to bind more than one FZD. Accordingly, in some embodiments, the antibody is one that specifically binds FZD4 and additionally specifically binds one or more of FZD 1, 2, 5, 7, 8 and 9. For example, the antibody can be an antibody wherein the CDR sequences are a CDR sequence set of an antibody selected from antibodies 5016, 5018-5023, 5025, 6495, 6496, 5039, 5045, 5048, 5054, 5056, 5057, 5067, and 5073-5076.

A number of the antibodies described preferentially bound FZD4. In an embodiment, the antibody is one that preferentially binds Frizzled 4 (FZD4) compared to any of FZD1, 2, 5, 7, 8, 9 or 10. In an embodiment, the antibody preferentially binds FZD 4 compared to FZD1, FZDS, FZD7 and FZD9. In an aspect, the antibody comprises the CDR sequence set of antibody 6497. In another embodiment, the antibody preferentially binds FZD4 compared to FZD1 and FZD7. In an aspect, the antibody comprises the CDR sequence set of an antibody selected from 5028, 5035, 5039, 5073. In yet another embodiment, the antibody preferentially binds FZD4 compared to FZD9. In an aspect, the antibody comprises the CDR sequence set of antibody 5029. In yet another embodiment, the antibody preferentially binds FZD4 compared to FZD1, FZD2 and FZD7. In an aspect the antibody comprises the CDR sequence set of antibody selected from 5031, 6498, 5054 or 5075. In yet another embodiment, the antibody preferentially binds FZD4 compared to FZD1, FZD2, FZD5 and FZD7. In an aspect, the antibody comprises the CDR sequence set of antibody 5034. In yet another embodiment, the antibody preferentially binds FZD4 compared to FZD1. In an aspect, the antibody comprises the CDR sequence set of antibody 5045 or 5048. In yet another embodiment, the antibody preferentially binds FZD4 compared to FZD1, FZD7 and FZD9. In an aspect, the antibody comprises the CDR sequence set of antibody 5056. In yet another embodiment, the antibody preferentially binds FZD4 compared to FZD9 and FZD10. In an aspect, the antibody comprises the CDR sequence set of antibody 5057. In yet another embodiment, the antibody preferentially binds FZD4 compared to FZD1 and FZD2. In an aspect, the antibody comprises the CDR sequence set of antibody 5067.

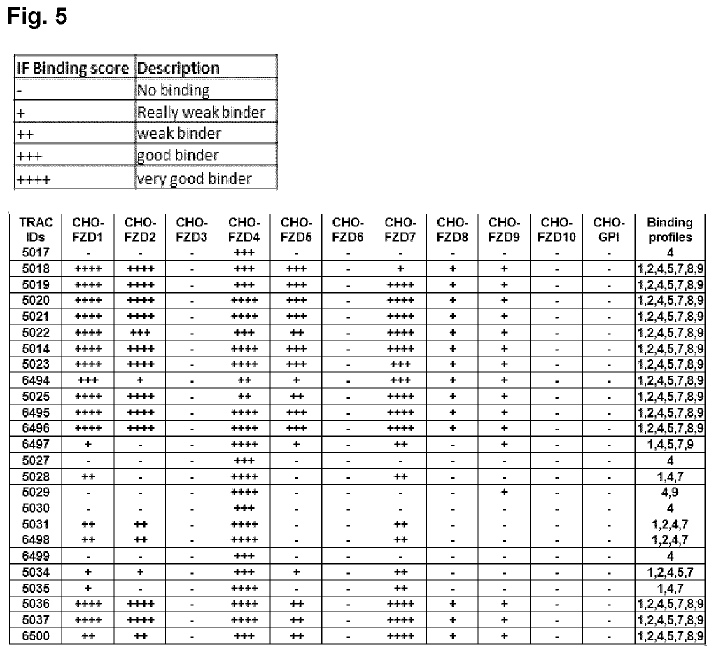

Certain antibodies, such as those with a CDR set from antibodies 5018, 5019, 5022, 6494, and 5025, preferentially bind other FZD proteins compared with FZD4. (See, for example, FIG. 5).

In another embodiment, the antibody comprises CDR sequences that are a CDR sequence set of an antibody selected from antibodies 5022, 5031, 6497, 6498 and 6500.

As demonstrated herein, the antibodies described herein have high affinity for FZD4. For example, the antibodies in one embodiment, have a binding affinity measured by surface plasmon resonance of between about 0.2 nM and about 15.3 nM.

The antibody can be a humanized antibody as described herein or a chimeric antibody.

In some embodiments, the antibody is a single chain antibody which can be obtained for example, by fusing the heavy chain and light chain or parts thereof together.

In some embodiments, the antibody is an antibody binding fragment selected from Fab, Fab′, F(ab′)2, scFv, dsFv, ds-scFv, dimers, nanobodies, minibodies, diabodies, and multimers thereof.

In some other embodiments the antibody is the binding fragment Fab. For some embodiments, the binding fragment is preferable.

It may be preferable in other embodiments to have a multivalent antibody or an antibody comprising an Ig portion.

As demonstrated in the Examples, an Fab fragment of the disclosure can be combined with an immunoglobulin (Ig) constant region such as an IgG. In an embodiment, the IgG is IgG1, IgG2, IgG3 or IgG4.

B. Detectably Labeled Antibodies

Detectable labels can include peptide sequences (such a myc tag, HA-tag, V5-tag or NE-tag), fluorescent or luminescent proteins (e.g., green fluorescent protein or luciferase) that can be appended to or introduced into an antibody described herein and which is capable of producing, either directly or indirectly, a detectable signal. For example, the label may be radio-opaque, positron-emitting radionuclide (for example, for use in PET imaging), or a radioisotope, such as ³H, ¹³N, ¹⁴C, ¹⁸F, ³²P, ³⁵S, ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion.

C. Antibody-Drug Conjugates

A further aspect includes an immunoconjugate comprising an antibody described herein and a detectable label or cytotoxic agent.

A chemotherapeutic (anti-cancer) agent can be any agent capable of reducing cancer growth, interfering with cancer cell replication, directly or indirectly killing cancer cells, reducing metastasis, reducing tumor blood supply, etc. Chemotherapeutic agents thus include cytotoxic agents. Cytotoxic agents include but are not limited to saporin, taxanes, vinca alkaloids, anthracycline, and platinum-based agents. Classes of chemotherapeutic agents include but are not limited to alkylating agents, antimetabolites (e.g., methotrexate), plant alkaloids (e.g., vincristine), and antitumor antibiotics such as anthracyclines (e.g., doxorubicin) as well as miscellaneous drugs that do not fall in to a particular class such as hydroxyurea. Platinum-based drugs, exemplified by cisplatin and oxaliplatin, represent a major class of chemotherapeutics. These drugs bind to DNA and interfere with replication. Taxanes, exemplified by taxol, represent another major class of chemotherapeutics. These compounds act by interfering with cytoskeletal and spindle formation to inhibit cell division, and thereby prevent growth of rapidly dividing cancer cells. Other chemotherapeutic drugs include hormonal therapy. Chemotherapeutics also include agents that inhibit tubulin assembly or polymerization such as maytansine, mertansine, and auristatin. Chemotherapeutic agents also include DNA damage agents such as calicheamicin.

Chemotherapeutic agents can include maytansinoid, auristatin, dolastatin, tubulysin, cryptophycin, pyrrolobenzodiazepine (PBD) dimer, indolinobenzodiazepine dimer, alpha-amanitin, trichothene, SN-38, duocarmycin, CC1065, calicheamincin, an enediyne antibioatic, taxane, doxorubicin derivatives, anthracycline and stereoisomers, azanofide, isosteres, analogs or derivatives thereof.

### IV. Nucleic Acids

Further aspects include nucleic acid molecules or polynucleotides, recombinant nucleic acid molecules, expression constructs, and vectors as described herein.

A. Nucleic Acid Molecules

A further aspect includes a nucleic acid molecule as set forth in Tables 1b, 1c, 3b and 3c as well as a polynucleotide that hybridizes to one of said sequences, for example, under stringent hybridization conditions. The CDR and variable domain nucleic sequences can be used for example, to prepare expression constructs.

B. Expression Constructs and Vectors

The nucleic acid molecules may be incorporated in a known manner into an appropriate expression construct or expression vector which ensures expression of the protein. Expression constructs can comprise an expression control sequence, e.g., a promoter, operatively linked with a polynucleotide comprising a nucleotide sequence encoding an antibody of this disclosure. Possible expression vectors include but are not limited to cosmids, plasmids, or modified viruses (e.g., replication defective retroviruses, adenoviruses and adeno-associated viruses). The vector should be compatible with the host cell used. The expression vectors are “suitable for transformation of a host cell”, which means that the expression vectors contain a nucleic acid molecule encoding the peptides corresponding to epitopes or antibodies described herein.

In an embodiment, the vector is suitable for expressing for example, single chain antibodies by gene therapy. In an embodiment, the vector comprises an IRES and allows for expression of a light chain variable region and a heavy chain variable region. Such vectors can be used to deliver antibody in vivo.

Suitable regulatory sequences may be derived from a variety of sources, including bacterial, fungal, viral, mammalian, or insect genes.

Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector.

In an embodiment, the regulatory sequences direct or increase expression in neural tissue and/or cells.

The vector can be any vector, including vectors suitable for producing an antibody described herein.

In an embodiment, the vector is a viral vector.

The recombinant expression vectors may also contain a marker gene which facilitates the selection of host cells transformed, infected or transfected with a vector for expressing an antibody or epitope peptide described herein.

The recombinant expression vectors may also contain expression cassettes which encode a fusion moiety (i.e. a “fusion protein”) which provides increased expression or stability of the recombinant peptide; increased solubility of the recombinant peptide; and aid in the purification of the target recombinant peptide by acting as a ligand in affinity purification, including for example, tags and labels described herein. Further, a proteolytic cleavage site may be added to the target recombinant protein to allow separation of the recombinant protein from the fusion moiety subsequent to purification of the fusion protein. Typical fusion expression vectors include pGEX (Amrad Corp., Melbourne, Australia), pMAL (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

Systems for the transfer of genes both in vitro and in vivo include vectors based on viruses, most notably Herpes Simplex Virus, Adenovirus, Adeno-associated virus (AAV) and retroviruses including lentiviruses. Alternative approaches for gene delivery include the use of naked, plasmid DNA as well as liposome—DNA complexes.

In an aspect the disclosure includes a method for making an antibody described herein, the method comprising synthesizing a nucleic acid molecule that comprises an antibody framework and a CDR sequence set described herein.

### V. Recombinant Cells

A further aspect is a recombinant host cell expressing an antibody described herein.

Antibodies as described herein can be made by recombinant expression of nucleic acids encoding the antibody sequences.

Antibodies as disclosed herein can be made by culturing cells engineered to express nucleic acid constructs encoding immunoglobulin polypeptides.

The recombinant host cell can be generated using any cell suitable for producing a polypeptide, for example, suitable for producing an antibody. For example, to introduce a nucleic acid (e.g., a vector) into a cell, the cell may be transfected, transformed or infected, depending upon the vector employed.

Suitable host cells include a wide variety of prokaryotic and eukaryotic host cells. For example, the proteins described herein may be expressed in bacterial cells such as E. coli, insect cells (using baculovirus), yeast cells or mammalian cells.

In an embodiment, the cell is a eukaryotic cell selected from a yeast, plant, worm, insect, avian, fish, reptile and mammalian cell.

In another embodiment, the mammalian cell is a CHO cell, a myeloma cell, a spleen cell, or a hybridoma cell.

Yeast and fungi host cells suitable for expressing an antibody include, but are not limited to Saccharomyces cerevisiae, Schizosaccharomyces pombe, the genera Pichia or Kluyveromyces and various species of the genus Aspergillus. Examples of vectors for expression in yeast S. cerivisiae include pYepSec1, pMFa, pJRY88, and pYES2 (Invitrogen Corporation, San Diego, Calif.). Protocols for the transformation of yeast and fungi are well known to those of ordinary skill in the art.

Mammalian cells that may be suitable include, among others: COS (e.g., ATCC No. CRL 1650 or 1651), BHK (e.g., ATCC No. CRL 6281), CHO (ATCC No. CCL 61), HeLa (e.g., ATCC No. CCL 2), 293 (ATCC No. 1573) and NS-1 cells. Suitable expression vectors for directing expression in mammalian cells generally include a promoter (e.g., derived from viral material such as polyoma, Adenovirus 2, cytomegalovirus and Simian Virus 40), as well as other transcriptional and translational control sequences. Examples of mammalian expression vectors include pCDM8 and pMT2PC.

### VI. Pharmaceutical Compositions

A further aspect is a composition comprising an antibody, immunoconjugate, nucleic acid molecule, vector or recombinant cell described herein, optionally with a suitable diluent, e.g., a pharmaceutically acceptable carrier.

The composition can for example, comprise one or more antibodies or immunoconjugates.

Suitable diluents for polypeptides, including antibodies and/or cells include but are not limited to saline solutions, pH buffered solutions and glycerol solutions or other solutions suitable for freezing polypeptides and/or cells.

Suitable diluents for nucleic acids include but are not limited to water, saline solutions and ethanol.

In an embodiment, the composition is a pharmaceutical composition comprising any of the antibodies, nucleic acids or vectors disclosed herein, and optionally comprising a pharmaceutically acceptable vehicle such as a diluent or carrier.

The compositions described herein can be prepared by per se known methods for the preparation of pharmaceutically acceptable compositions that can be administered to subjects, such that an effective quantity of the active substance is combined in a mixture with a pharmaceutically acceptable vehicle.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween), alcohols, polyols, glycerin and vegetable oils, for example. Extemporaneous injection solutions and suspensions may be prepared from sterile powders, granules, tablets, or concentrated solutions or suspensions. The composition may be supplied, for example, but not by way of limitation, as a lyophilized powder which is reconstituted with sterile water or saline prior to administration to the patient.

Pharmaceutical compositions may comprise a pharmaceutically acceptable carrier. Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy)propyl)N,N,N-trimethylammonium chloride (DOTMA), diolesylphosphotidyl-ethanolamine (DOPE), and liposomes. Such compositions should contain a therapeutically effective amount of the compound, together with a suitable amount of carrier so as to provide the form for direct administration to the patient.

The composition may be in the form of a pharmaceutically acceptable salt which includes, without limitation, those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylarnino ethanol.

In an embodiment, the composition comprises an antibody described herein. In another embodiment, the composition comprises an antibody described herein and a diluent. In an embodiment, the composition is a sterile composition.

A further aspect includes an antibody complex comprising an antibody described herein bound to an FZD protein, e.g., FZD4. The complex may be in solution or comprised in a tissue, optionally in vitro.

Also provided are methods for making and using the regents described herein.

### VII. Methods of Administration and Use

The anti-FZD antibodies of the invention can efficiently deliver a therapeutic composition to cells undergoing Wnt signaling in vivo. In some embodiments, the method of treatment or use comprises administering to an individual or use of an effective amount of a therapeutic anti-FZD conjugate, e.g., an anti-FZD antibody attached to a therapeutic agent. In some embodiments, the individual has been diagnosed with cancer. In some embodiments, the individual is receiving or has received cancer therapy, e.g., surgery, radiotherapy, or chemotherapy. In some embodiments, the individual has been diagnosed, but the cancer is in remission.

In some embodiments, the anti-FZD conjugate includes a liposome. In some embodiments, the method further comprises monitoring the individual for progression of the cancer. In some embodiments, the dose of the anti-LRP conjugate for each administration is determined based on the therapeutic progress of the individual, e.g., where a higher dose of chemotherapeutic is administered if the individual is not responding sufficiently to therapy.

In some embodiments, the invention can include an antibody or antibody-targeted composition and a physiologically (i.e., pharmaceutically) acceptable carrier. The term “carrier” refers to a typically inert substance used as a diluent or vehicle for a diagnostic or therapeutic agent. The term also encompasses a typically inert substance that imparts cohesive qualities to the composition. Physiologically acceptable carriers can be liquid, e.g., physiological saline, phosphate buffer, normal buffered saline (135-150 mM NaCl), water, buffered water, 0.4% saline, 0.3% glycine, glycoproteins to provide enhanced stability (e.g., albumin, lipoprotein, globulin, etc.), and the like. Since physiologically acceptable carriers are determined in part by the particular composition being administered as well as by the particular method used to administer the composition, there are a wide variety of suitable formulations of pharmaceutical compositions of the present invention (See, e.g., Remington's Pharmaceutical Sciences, 17th ed., 1989).

The compositions of the present invention may be sterilized by conventional, well-known sterilization techniques or may be produced under sterile conditions. Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents, wetting agents, and the like, e.g., sodium acetate, sodium lactate, sodium chloride, potassium chloride, calcium chloride, sorbitan monolaurate, and triethanolamine oleate. Sugars can also be included for stabilizing the compositions, such as a stabilizer for lyophilized antibody compositions.

Dosage forms can be prepared for mucosal (e.g., nasal, sublingual, vaginal, buccal, or rectal), parenteral (e.g., subcutaneous, intravenous, intramuscular, or intraarterial injection, either bolus or infusion), oral, or transdermal administration to a patient. Examples of dosage forms include, but are not limited to: dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

Injectable (e.g., intravenous) compositions can comprise a solution of the antibody or antibody-targeted composition suspended in an acceptable carrier, such as an aqueous carrier. Any of a variety of aqueous carriers can be used, e.g., water, buffered water, 0.4% saline, 0.9% isotonic saline, 0.3% glycine, 5% dextrose, and the like, and may include glycoproteins for enhanced stability, such as albumin, lipoprotein, globulin, etc. Often, normal buffered saline (135-150 mM NaCl) will be used. The compositions can contain pharmaceutically acceptable auxiliary substances to approximate physiological conditions, such as pH adjusting and buffering agents, tonicity adjusting agents, wetting agents, e.g., sodium acetate, sodium lactate, sodium chloride, potassium chloride, calcium chloride, sorbitan monolaurate, triethanolamine oleate, etc. In some embodiments, the antibody-targeted composition can be formulated in a kit for intravenous administration.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Injection solutions and suspensions can also be prepared from sterile powders, granules, and tablets. In the practice of the present invention, compositions can be administered, for example, by intravenous infusion, topically, intraperitoneally, intravesically, or intrathecally. Parenteral administration and intravenous administration are the preferred methods of administration. The formulations of targeted compositions can be presented in unit-dose or multi-dose sealed containers, such as ampoules and vials.

The targeted delivery composition of choice, alone or in combination with other suitable components, can be made into aerosol formulations (“nebulized”) to be administered via inhalation. Aerosol formulations can be placed into pressurized acceptable propellants, such as dichlorodifluoromethane, propane, and nitrogen.

The pharmaceutical preparation can be packaged or prepared in unit dosage form. In such form, the preparation is subdivided into unit doses containing appropriate quantities of the active component, e.g., according to the dose of the therapeutic agent or concentration of antibody. The unit dosage form can be a packaged preparation, the package containing discrete quantities of preparation. The composition can, if desired, also contain other compatible therapeutic agents.

The antibody (or antibody-targeted composition) can be administered or for use by injection or infusion through any suitable route including but not limited to intravenous, subcutaneous, intramuscular or intraperitoneal routes. An example of administration of a pharmaceutical composition includes storing the antibody at 10 mg/ml in sterile isotonic aqueous saline solution for injection at 4° C., and diluting it in either 100 ml or 200 ml 0.9% sodium chloride for injection prior to administration to the patient. The antibody is administered by intravenous infusion over the course of 1 hour at a dose of between 0.2 and 10 mg/kg. In other embodiments, the antibody is administered by intravenous infusion over a period of between 15 minutes and 2 hours. In still other embodiments, the administration procedure is via sub-cutaneous bolus injection.

The dose of antibody is chosen in order to provide effective therapy for the patient and is in the range of less than 0.1 mg/kg body weight to about 25 mg/kg body weight or in the range 1 mg-2 g per patient. In some cases, the dose is in the range 1-100 mg/kg, or approximately 50 mg-8000 mg/patient. The dose may be repeated at an appropriate frequency which may be in the range once per day to once every three months, depending on the pharmacokinetics of the antibody (e.g., half-life of the antibody in the circulation) and the pharmacodynamic response (e.g., the duration of the therapeutic effect of the antibody). In some embodiments, the in vivo half-life of between about 7 and about 25 days and antibody dosing is repeated between once per week and once every 3 months.

Administration or use can be periodic. Depending on the route of administration, the dose can be administered, e.g., once every 1, 3, 5, 7, 10, 14, 21, or 28 days or longer (e.g., once every 2, 3, 4, or 6 months). In some cases, administration is more frequent, e.g., 2 or 3 times per day. The patient can be monitored to adjust the dosage and frequency of administration depending on therapeutic progress and any adverse side effects, as will be recognized by one of skill in the art.

Thus, in some embodiments, additional administration is dependent on patient progress, e.g., the patient is monitored between administrations. For example, after the first administration or round of administrations, the patient can be monitored for rate of tumor growth, recurrence (e.g., in the case of a post-surgical patient), or general disease-related symptoms such as weakness, pain, nausea, etc.

In therapeutic use for the treatment of cancer, an antibody-targeted composition (e.g., including a therapeutic and/or diagnostic agent) can be administered at the initial dosage of about 0.001 mg/kg to about 1000 mg/kg daily and adjusted over time. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosage is varied depending upon the requirements of the patient, the severity of the condition being treated, and the targeted composition being employed. For example, dosages can be empirically determined considering the type and stage of cancer diagnosed in a particular patient. The dose administered to a patient, in the context of the present invention, should be sufficient to affect a beneficial therapeutic response in the patient over time. The size of the dose will also be determined by the existence, nature, and extent of any adverse side-effects that accompany the administration of a particular targeted composition in a particular patient, as will be recognized by the skilled practitioner.

### VIII. Kits

Another aspect is a kit or package comprising any of the antibodies, immunoconjugates, nucleic acid molecules, vectors, recombinant cells and/or compositions comprised herein. The antibodies, immunoconjugates, nucleic acid molecules, vectors, recombinant cells and/or compositions can be comprised in a vial such as a sterile vial or other housing. As used herein, the term “kit” refers to a collection of items intended for use together. The kit can optionally include a reference agent and/or instructions for use thereof. A kit can further include a shipping container adapted to hold a container, such as a vial, that contains a composition as disclosed herein.

### IX. Methods of Using Antibodies

Antibodies described herein can be used in a number of in vitro and in vivo methods.

A. Methods of Detecting Expression of FZD

As demonstrated herein, the antibodies can be used to detect FZD expression.

Accordingly, the disclosure provides in one aspect, a method of detecting FZD expression, the method comprising contacting a sample comprising one or more cells with one or more antibody or immunoconjugates described herein under conditions permissive for forming an antibody:FZD complex and detecting the presence of any antibody complex. Typically, the antibody is part of an immunoconjugate comprising an antibody coupled to a detectable label.

The sample can comprise viable cells or a cell extract. The antibody: FZD complex can be detected immunoassays such as immunofluorescence, flow cytometry, Western blots, ELISA, SPR and immunoprecipitation followed by SDS-PAGE immunocytochemistry. In some embodiments, the detection is by immunofluorescence. In some embodiments, the detection is by flow cytometry.

As demonstrated herein, a number of the antibodies identified preferentially recognize FZD4. Accordingly, in embodiments wherein the method is for detecting FZD4 expression, the antibody or immunoconjugate comprises a CDR sequence set corresponding to an antibody selected from 5017, 5027, 5030, 6499, 5038, 5040-5044, 5046, 5047, 5049-5053, 5055, 5058-5064, 5066, 5068-5072, and 5077-5081.

B. Methods of Inhibiting WNT Binding to FZD

Antibodies disclosed herein inhibit binding of Wnt to Frizzled receptors, in particular, to FZD4. Without wishing to be limited by theory, inhibition of Wnt binding to FZD proteins impacts signal transduction of which FZD plays a role in initiating. For example, antibody binding to FZD receptors inhibits FZD promotion of beta-catenin phosphorylation. Beta-catenin that is not phosphorylated will escape destruction in a cell and accumulate. Accumulation of beta-catenin is associated with malignancy.

It can be desirable to reduce or inhibit Wnt ligand signaling through FZD. Accordingly, another aspect is a method of inhibiting Wnt ligand binding to a FZD or Wnt induced transcriptional activity comprising contacting one or more cells expressing one or more FZD polypeptides with an effective amount of an antibody or immunoconjugate described herein.

In an embodiment, the antibody or immunoconjugate comprises a CDR sequence set (full, light chain or heavy chain) corresponding to an antibody selected from a clone as described herein, e.g., 5014, 5017-5023, 5027-5031, 5034, 5036, 5037, 6496, 6498, 6499 and 6500, 5035, 6495 and 5025.

In an embodiment, the antibody or immunoconjugate comprises a CDR sequence set (full, light chain or heavy chain) corresponding to an antibody selected from a clone as described herein, e.g., 5014, 5018-5023, 5025, 5036, 5037, 6495, 5027-5031 and 6497-6499.

The contacting can for example, be done in vivo by administering an antibody or immunoconjugate to the subject. Such inhibition may be desirable particularly where wnt signaling is dysregulated as in cancer cells.

C. Methods of Treating Cancer

Methods of treating cancer comprise administering to a subject in need thereof a pharmaceutical composition comprising an antibody of this disclosure that binds to FZD. The subject in thereof can be a subject, e.g., a person, suffering from cancer, or at risk of cancer, such as recurrence of cancer.

Without wishing to be limited by theory, such therapy may function by inhibiting activation of the canonical Wnt pathway, for example, by inhibiting Wnt binding to FZD, by inhibiting Wnt-induced transcriptional activity, by inhibiting activation of disheveled, by inhibiting inhibition of the beta-catenin destruction complex and by promoting accumulation of beta-catenin.

The disclosure in another aspect includes a method for treating cancer, the method comprising administering an effective amount of an antibody or immunoconjugate that specifically binds FZDs 1, 2, 4, 5, 7, 8 and 9 in at least one assay, and inhibits Wnt-induced signalling in at least one assay to a subject in need thereof. The disclosure also includes an effective amount of an antibody or immunoconjugate that specifically binds FZDs 1, 2, 4, 5, 7, 8 and 9 in at least one assay, and inhibits Wnt-induced signalling in at least one assay for use in treating cancer. The disclosure also provides a use of an effective amount of an antibody or immunoconjugate that specifically binds FZDs 1, 2, 4, 5, 7, 8 and 9 in at least one assay, and inhibits Wnt-induced signalling in at least one assay for treating cancer. The disclosure yet also provides a use of an effective amount of an antibody or immunoconjugate that specifically binds FZDs 1, 2, 4, 5, 7, 8 and 9 in at least one assay, and inhibits Wnt-induced signalling in at least one assay in the manufacture of a medicament for treating cancer.

In an embodiment, antibody or immunoconjugate, e.g., an antibody-drug conjugate, is comprised in a pharmaceutical composition.

In an embodiment, the cancer is selected from colon, lung, breast ovarian, endometrial, pancreas, stomach, liver, adrenocortical carcinoma and osteoblastoma cancer, optionally the cancer is pancreatic cancer. In an embodiment, the antibody or immunoconjugate comprises a CDR sequence set (full, light chain or heavy chain) corresponding to an antibody selected from 5014, 5017-5023, 5025, 5035-5037, 6495 and 6500.

As demonstrated herein, the antibodies are also able to inhibit cancer cell proliferation. Accordingly, also provided is a method for inhibiting cancer cell proliferation comprising contacting one or more cancer cells expressing an FZD with an effective amount of an antibody or immunoconjugate that specifically binds FZDs 1, 2, 4, 5, 7, 8 and 9 in at least one assay, and inhibits Wnt3a-induced signalling in at least one assay.

In an embodiment, the antibody or immunoconjugate is the antibody or immunoconjugate described herein, for example, an antibody or immunoconjugate that comprises a CDR sequence set (full, light chain or heavy chain) corresponding to an antibody selected from 5014, 5017-5023, 5025, 5035-5037, 6495 and 6500.

In an embodiment, the antibody comprises a variable region sequence as described herein a CDR sequence set (full, light chain or heavy chain) corresponding to an antibody selected from 5014, 5017-5023, 5025, 5035-5037, 6495 and 6500.

In one embodiment, the cancer is selected from acute myeloid leukemia, prostate cancer, glioblastoma, bladder cancer and cervical cancer.

In another embodiment, the cancer cells are selected from colon, lung, breast ovarian, endometrial, pancreas, stomach, liver, adrenocortical carcinoma and osteoblastoma cancer cells.

In another embodiment, the cancer cells are pancreatic cancer cells. In an embodiment, the antibody or immunoconjugate comprises a CDR sequence set (full, light chain or heavy chain) corresponding to an antibody selected from 5019 and 5020.

It is also demonstrated that antibody 5020 is efficacious for treating RNF43 mutated cancers. Accordingly, in one embodiment, cancer cells known or determined to comprise a mutation in RNF43 gene, and the antibody or immunoconjugate used comprises a set of CDRs corresponding to antibody 5020.

In an embodiment, the method includes determining that a cancer in a subject is associated with Wnt signalling dysregulation; optionally determining the specific Wnt protein that is dysregulated; optionally determining the member of the FZD protein family to be targeted; and administering to the subject and anti-FZD antibody to block selected one or more Wnt binding to selected one or more FZD receptors.

The above disclosure generally describes the present disclosure. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

### X. Examples

### Example 1

### Antibody Selection and Functional Testing of the FZD4 Fabs

Antibody selection: Two selection approaches were undertaken to identify FZD4 binders.

1. Selection was done using libraries designed based on previous FZD7 derived binders. Antibodies have been previously identified from selection using FZD7 CRD-Fc as antigen. These antibodies bind to FZD1, 2, 5, 7, 8 and 9 and showed antagonistic activities against Wnt pathway and inhibit proliferation and tumor growth of pancreatic cancer cells. The library was used to identify antibodies that would bind to FZD4 and have wnt-antagonistic and anti-tumor activity.

a). Fab phage display library design and preparation.

Fab phage display library design and preparation was done with IPTG inducible display vector encoding Fab that recognizes MBP. Fab template is identical to library F and includes FLAG tagged light chain and dimerization domain L1, L2, L3 mutated to the parental Fab sequence H1, H2, and H3 were soft randomized to allow for a 50% bias towards the wild-type amino acid and 50% any other amino acid (using a 70:10:10:10 nucleotide mix). All six CDR regions were mutated in a single kunkel mutagenesis reaction. Second generation libraries were constructed based on the Fab antagonist panel. An IPTG inducible display vector encoding a Fab specific for maltose binding protein was used as the library template. Site-specific kunkel mutagenesis reactions were carried out using light chain oligos to mutate CDRs L1, L2 and L3 to the parental Fab sequence and soft randomize (50% wildtype and 50% any other amino acid) CDRs H1, H2, and H3. Purified mutagenesis reactions were electroporated into SR320 cells pre-infected with M13 K07. Libraries were rescued in 500 ml cultures overnight, double precipitated with PEG/NaCl, and resuspended in PBS with 50% glycerol for storage at −20 degrees C.

b). Selection and generation of Fabs against FZD4

Second generation libraries were pooled (equal cfu) and screened for four rounds against the recombinant FZD4 cysteine rich domain (CRD) fused to an Fc tag (R&D systems). Input and output phage titers were calculated for carbenecillin (carb) resistant library phage and kanamycin (kan) resistant helper phage. Maxisorb plates were coated overnight at 4 degrees with 5 μg/ml of FZD4-CRD-Fc or Fc protein in PBS and indicated number of wells, and blocked with 0.5% BSA. Coated wells were washed four times with PBS/0.5% Tween 20 (wash buffer) and library phage (PEG precipitated and resuspended in 0.5% BSA/0.05%Tween20/PBS) was incubated for 1 hr, room temperature in the Fc protein wells first. Unbound phage were transferred to the blocked FZD4-CRD-Fc plates and incubated for 1 hr, RT. Wells were washed as indicated and then eluated with 100 mM HCl. Eluted phage were amplified using standard protocols in the lab for subsequent rounds of selection. Input and output titers of library phage (carb resistant) and helper phage (kan resistant) are indicated. DNA from a site-specific kunkel mutagenesis reaction, designed to add a His6-amber stop between the phage gene III and Fab CH1 region, of the output phage pools from FZD4 selections were transformed into Omnimax cells and plated for single colonies. Single colonies were used to inoculated 96-well culture boxes, and overnight phage supernatants were diluted 1:2 in 0.05% Tween20/0.5% BSA/PBS (dilution buffer) to test for ELISA binding. Phage were detected with an anti-M13-HRP secondary antibody (1:5000 in dilution buffer) and plates were developed with TMB substrate and an acid stop. The absorbance at 450 nm was read for FZD4-Fc coated wells and control Fc coated wells, as indicated. Heavy and light chains were sequenced to determine CDR sequences of individual Fabs. The CH1-geneIII junction was also sequenced to determine successful incorporation of the His tag and amber stop codon for Fab expression. Phage binders were cloned into a bacterial expression vector and purified as Fab proteins for characterization.

c). Characterization of anti-FZD4 binders.

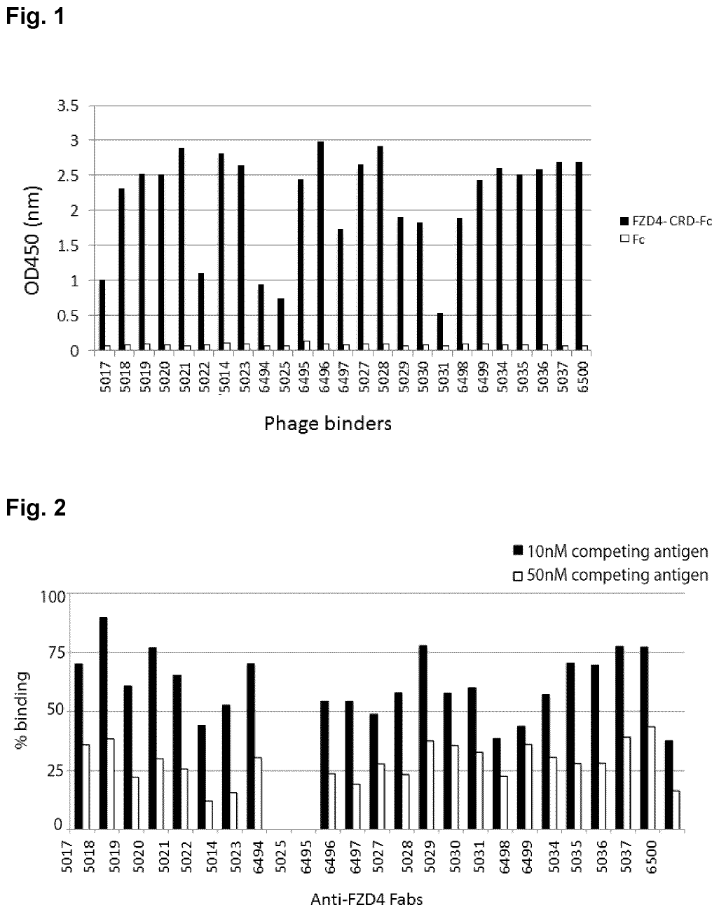

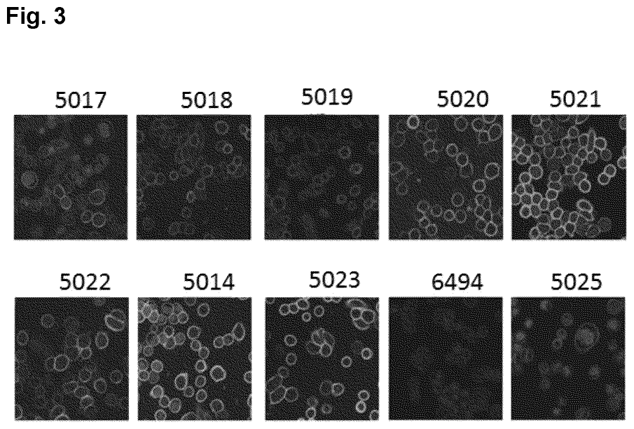

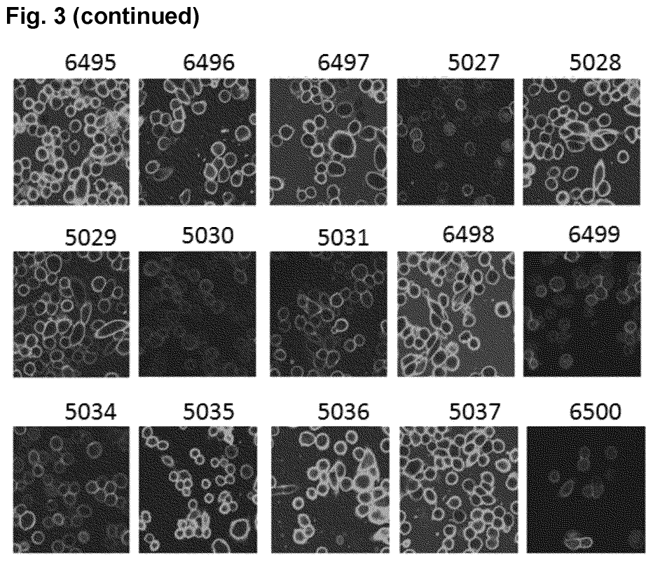

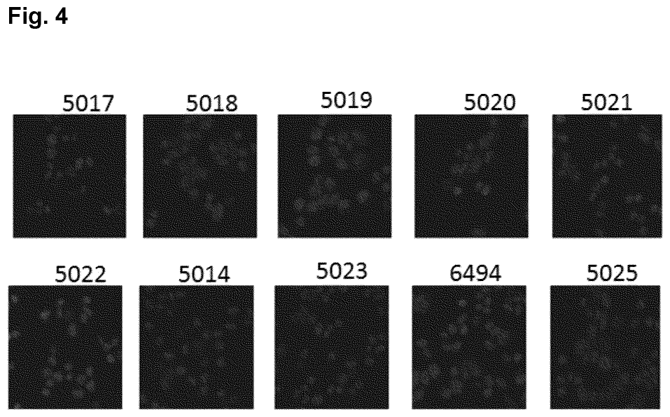

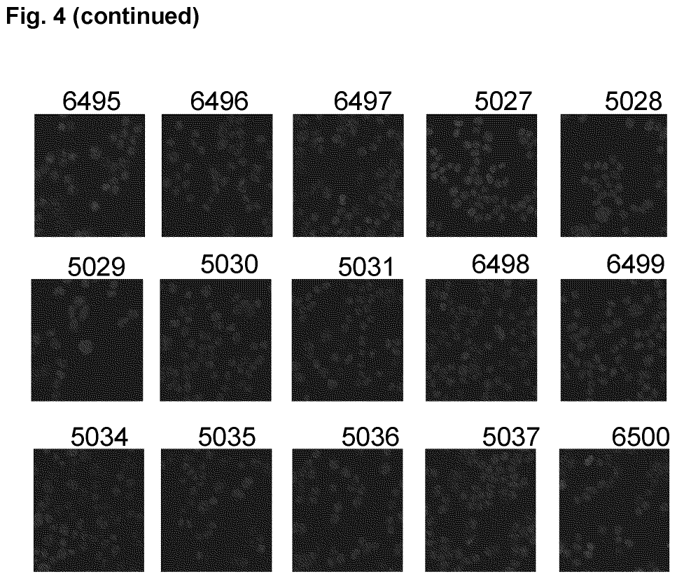

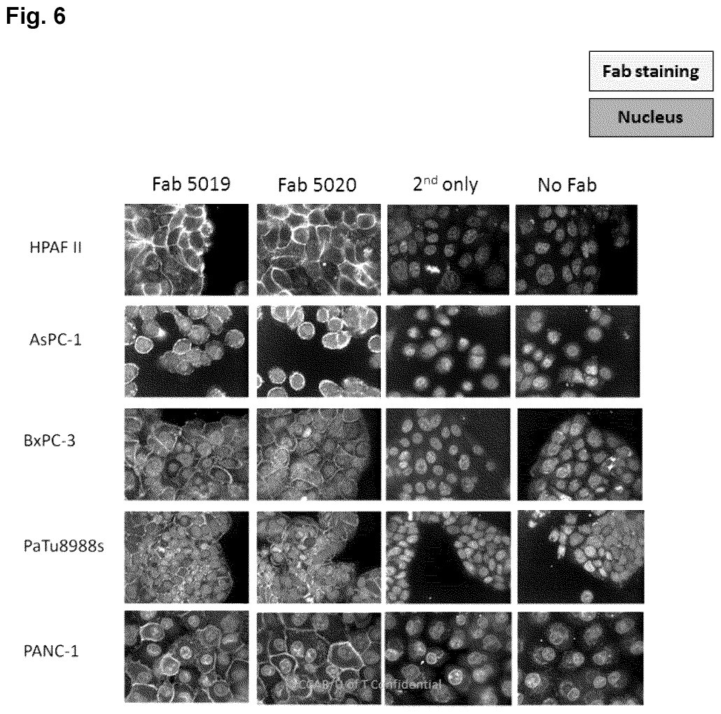

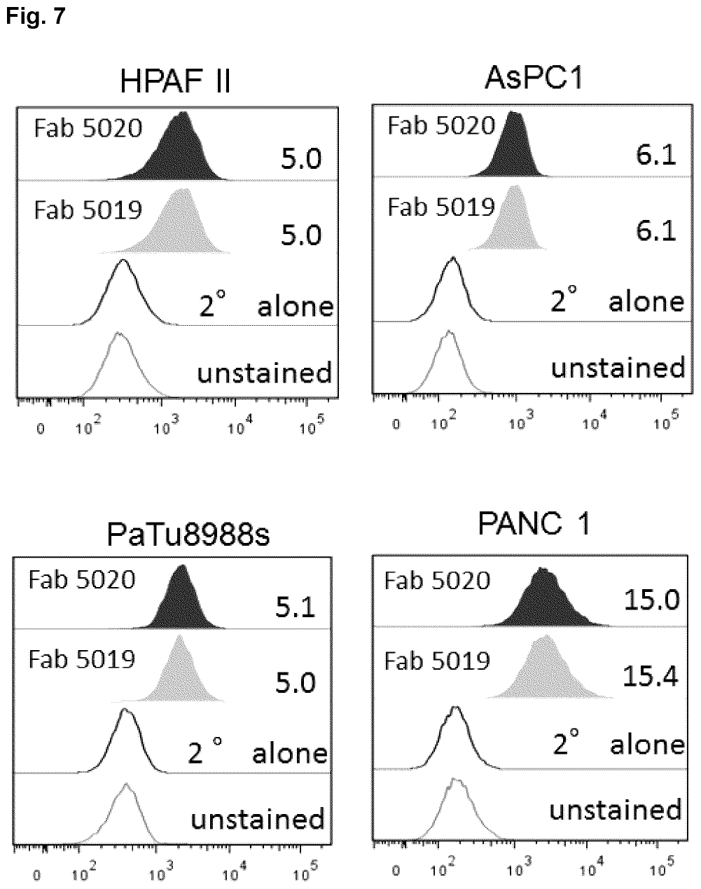

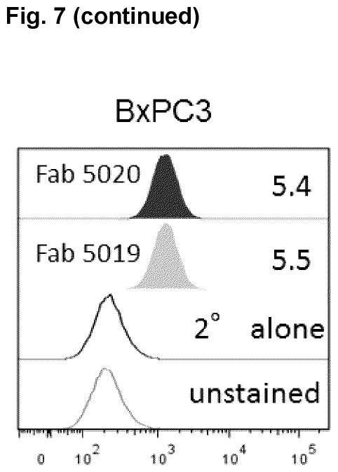

CDR Sequences of antibodies described here are shown in Table 1. First, the phage binders were tested in an ELISA assay to confirm their binding to the antigen. As shown in FIG. 1, all the phage clones bind to the FZD4 CRD-Fc, but not to Fc protein alone, suggesting these phage binders bind to FZD4 CRD not to Fc. Second, the purified Fabs were tested in a competitive ELISA assay in the presence of increasing amounts of non-immobilized antigen to estimate their binding affinity (FIG. 2). Binding of all Fabs was reduced by more than 50% in the presence of 50 nM of the competing free antigen (FZD4 CRD). Binding of five Fabs (5022, 5031, 6497, 6498 and 6500) was reduced by more than 50% in the presence of 10 nM of the competing antigen (FZD4 CRD) suggesting these 5 Fabs may have higher binding affinity than the rest. It should be noted that no binding was observed for Fabs 5025 and 6494 in this assay. The reason for this is unknown, but likely due to physical interference. Next the Fabs were tested for binding to FZD4 expressed on cell surface by immunofluorescent staining. Membrane staining pattern was observed for all the FZD4 Fabs on the CHO cells that stably express FZD4 (FIG. 3) but not on the CHO cells (FIG. 4). IF staining was also used to determine if these Fabs bind to other FZDs. The binding was tested on 10 CHO cell lines that stably express individual FZDs on the surface. As shown in FIG. 5, the Fabs showed various binding profiles. Fabs 5017, 5027, 5030 and 6499 bind to only FZD4-expressing CHO cells while the other Fabs (5014, 5018-5023, 5025, 6495, 6496) bind to multiple FZDs including FZDs 1,2,4,5,7,8,9. However no Fabs were shown to bind to FZD 3, 6 or 10 in this assay. Next, Fabs 5019 and 5020 were used as examples to test their binding to cancer cells by both immunofluorescent staining and flow cytometry. As shown in FIG. 6, both Fabs showed clear membrane-focused staining on 5 pancreatic cell lines tested. Binding of these Fabs to these cancer cell lines were confirmed by flow cytometry as shown in FIG. 7.

2. Selection using naïve Fab library (Library F).

a). Recombinant FZD4-CRD-Fc was used for selection of Fabs that bind to FZD4. Specifically, Fab phage library (Library F) was precleared from non-specific binders with a non-relevant protein. Several rounds of selection were performed against the precleared Fab library to enrich binders that bind to the FZD4-CRD-Fc. Clonal phages from the selection were screened by ELISA for binders specifically binding to FZD4-CRD-Fc but not to Fc. Forty-four Fabs with unique CDR sequences were identified (See table 3 for sequences).

b). Characterization of the anti-FZD4 Fabs

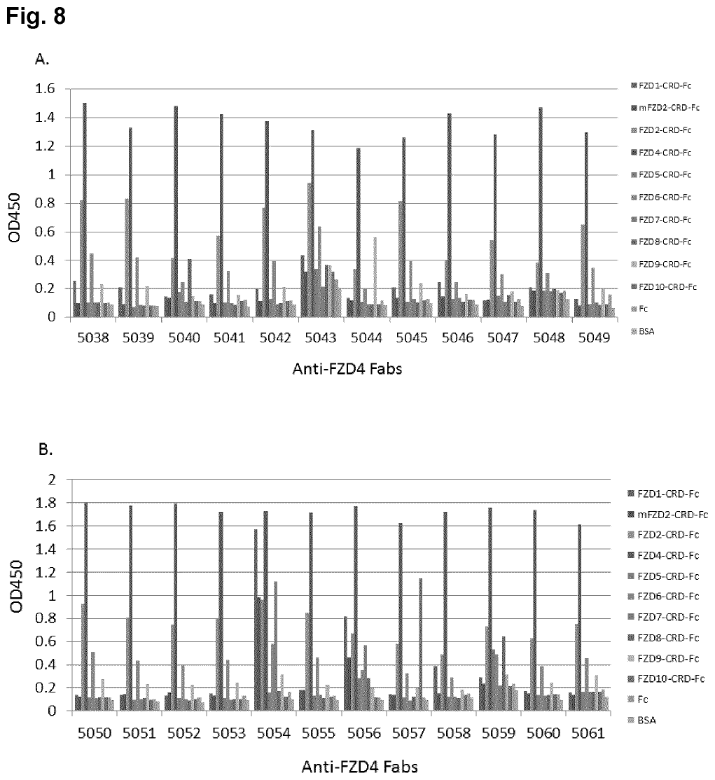

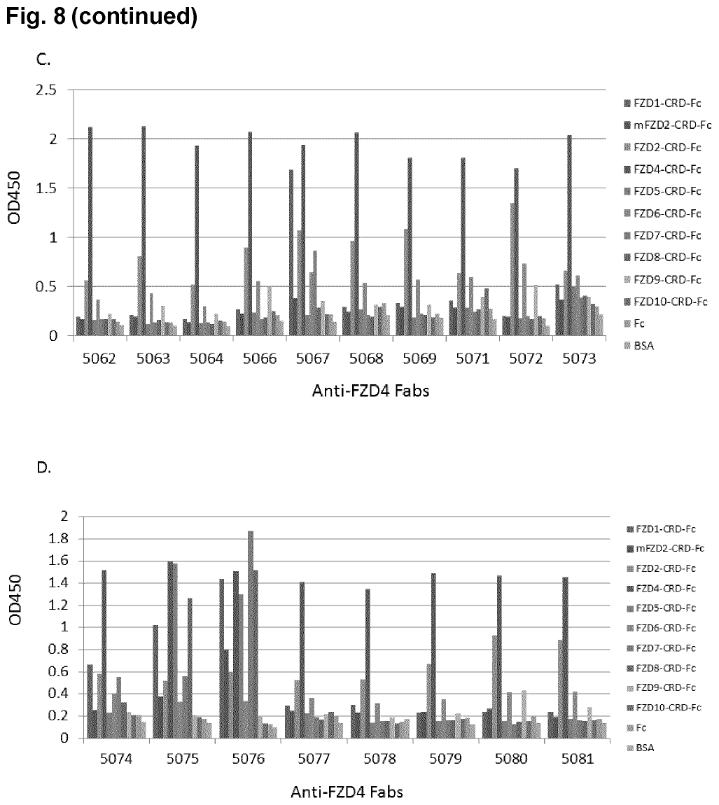

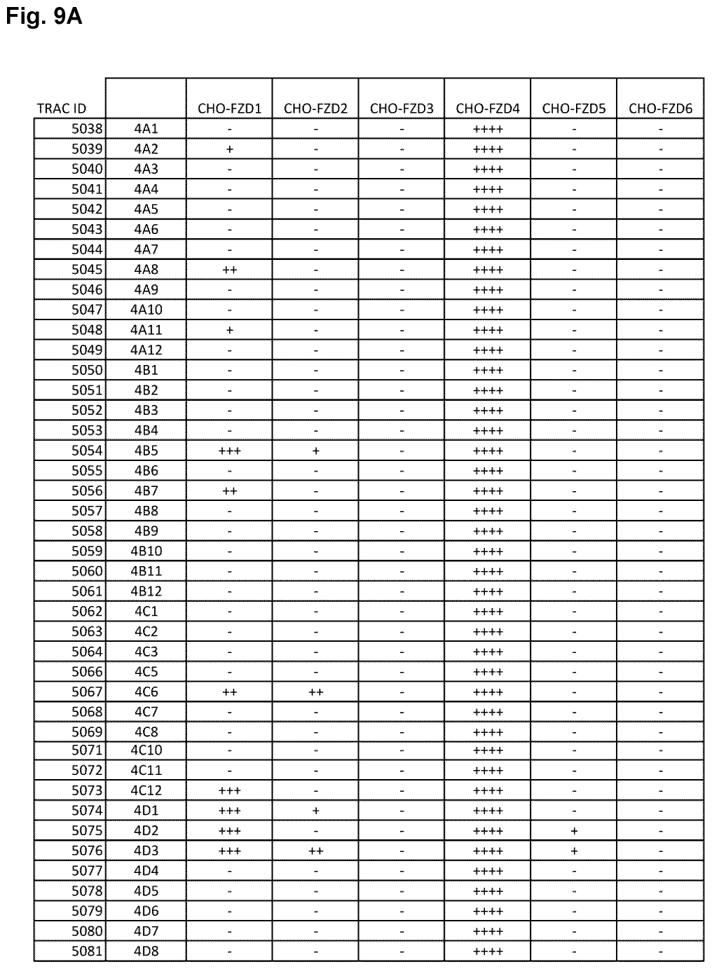

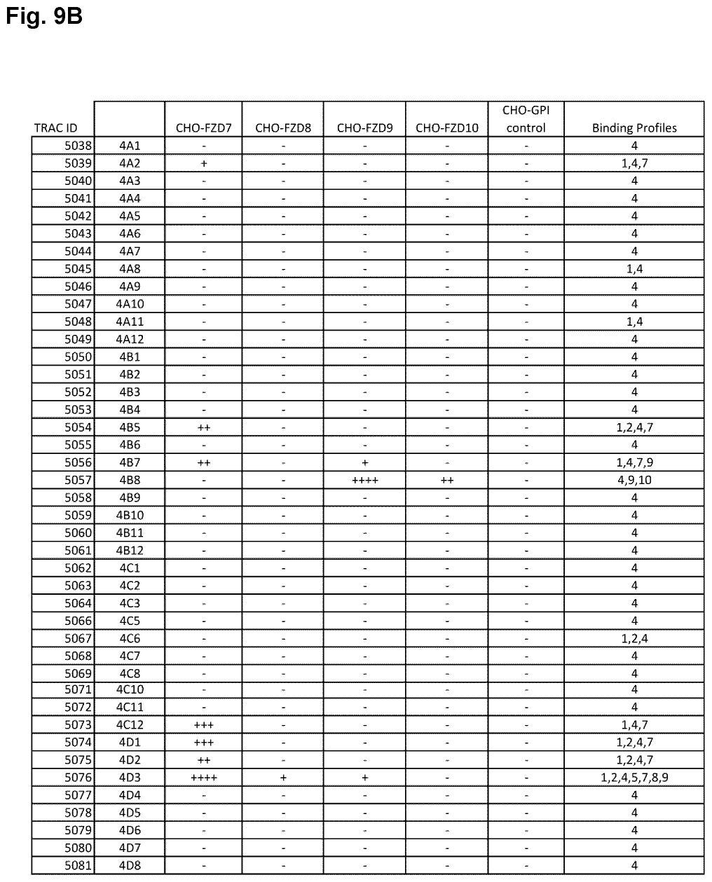

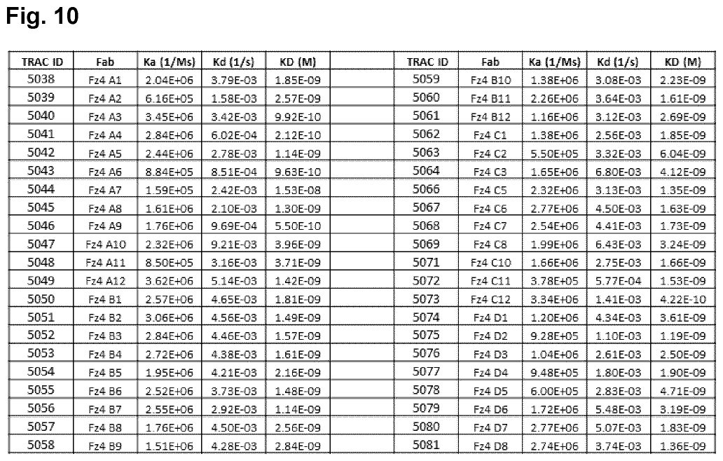

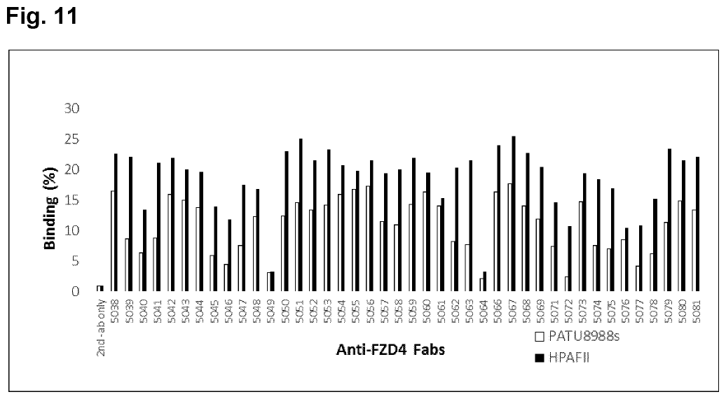

The anti-FZD4 phage binder clones were then cloned into a bacterial expression vector and 42 Fabs were expressed and purified for further characterization. Multiple methods were employed to determine the binding selectivity to FZDs. First, the purified Fabs were tested in an ELISA assay to confirm their binding to the recombinant antigen (FZD4-CRD-Fc) and their binding to other FZDs (FZD1, 2, 5, 6, 7, 8, 9 and 10). As shown in FIG. 8, all Fabs bind to FZD4-CRD-Fc (the antigen used for selection) but little binding to Fc or non-relevant protein BSA was observed. In addition, these Fabs were shown to also bind to other FZDs to varying degrees (FIG. 8). For example, small amounts of binding to FZD2 CRD-Fc and FZD8 CRD-Fc were detected for Fab in addition to its binding to FZD4-CRD-Fc (FIG. 8A). Fab 5076 binds to FZD1, 2, 5, 7 and 8 in addition to FZD4 (FIG. 8D). Next, the binding of the anti-FZD4 Fabs to various FZDs were determined by immunofluorescent staining of FZD expressing CHO cells and results are summarized in FIG. 9. Most of the Fabs were shown to bind to FZD4 only in this assay including Fabs 5038, 5040-5044, 5046, 5047, 5049-5053, 5055, 5058-5064, 5066, 5068-5072, 5077-5081) while the others to 2 or more FZDs. Further, surface plasmon resonance (SPR) was used to determine the binding affinity of the anti-FZD4 Fabs to FZD4 CRD-Fc. As shown in FIG. 10. The FZD4-derived Fabs show high affinity to FZD4 ranging from 0.2 nM to 15.3 nM. To see if these Fabs bind to FZDs expressed on pancreatic cancer cells, flow cytometry was used to test their binding. As summarized in FIG. 11, most Fabs were able to bind to HPAFII and PATU8988s. Small amounts of binding were seen for Fabs 5049, 5064 and 5072 (PATU8988s cells).

Functional testing of the FZD4 Fabs: several assays were performed to characterize the anti-FZD4 Fabs.

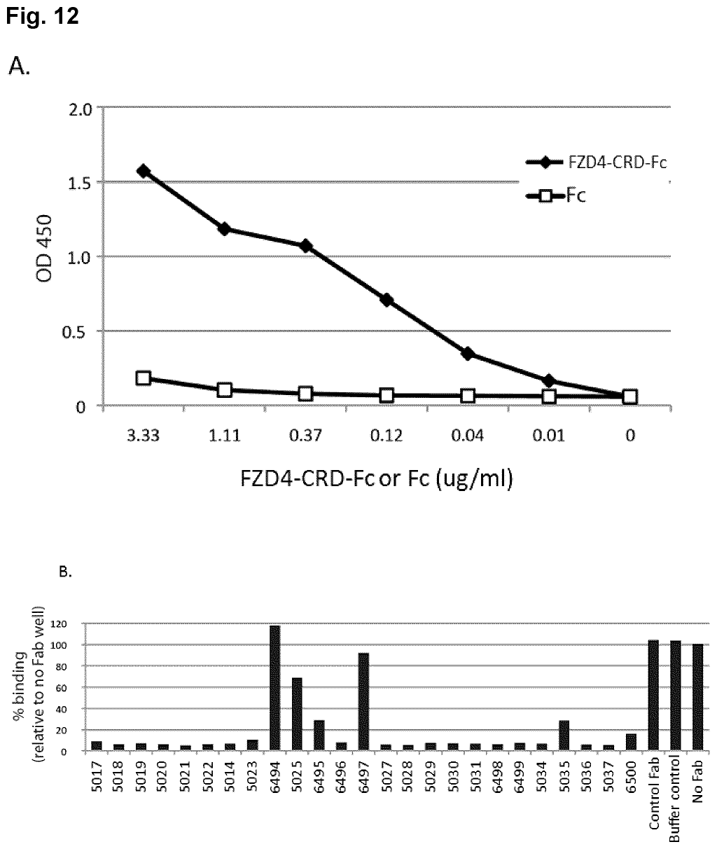

1. Effect of anti-FZD4 Fabs on Wnt ligand binding to FZD4-CRD. To optimize the assay conditions, increasing concentrations of FZD4-CRD-Fc or Fc were mixed with biotinylated Wnt5A and the complexes were captured by streptavidin coated plates. The binding of FZD4-CRD-Fc or Fc was detected by anti-Fc-HRP (FIG. 12A). A concentration of FZD4-CRD-Fc which gives an ELISA signal within a linear range was chosen for testing blocking activity of the anti-FZD4 Fabs. FZD4-CRD-Fc was mixed with biotinalyted Wnt5A in the presence of various Fabs as indicated and the bound FZD4-CRD-Fc was detected by anti-Fc-HRP as in FIG. 12A. As shown in FIG. 12B, greater than 80% inhibition of binding was observed for Fabs 5014, 5017-5023, 5027-5031, 5034, 5036, 5037, 6496, 6498, 6499 and 6500; greater than 60% inhibition of binding for Fabs 5035 and 6495; —30% inhibition for Fab 5025; and no inhibition was seen for Fabs 6494 and 6497.

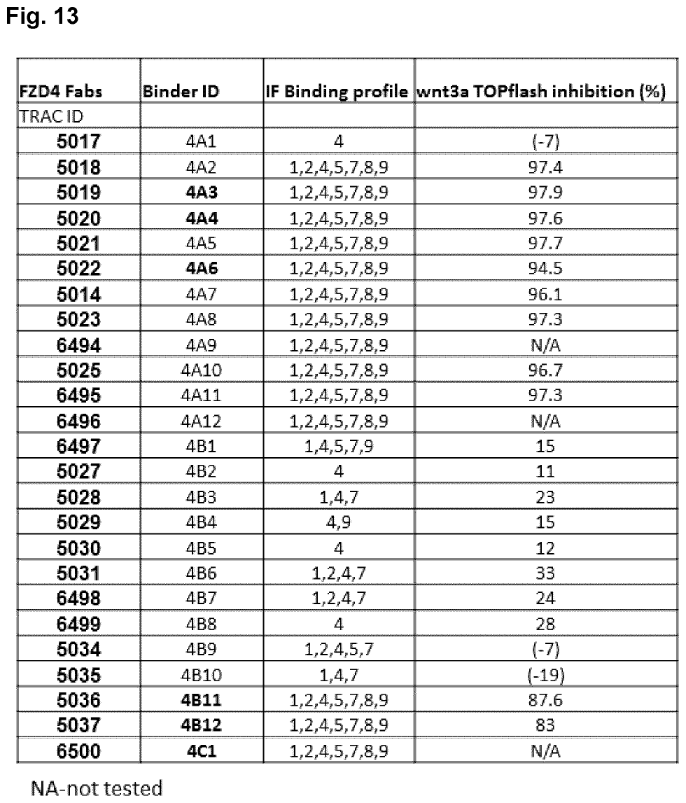

2. Effect of anti-FZD4 Fabs on beta-catenin driven transcription. To see if the anti-FZD4 Fabs affect beta-catenin dependent signalling, the Fabs were tested for their effect on Wnt3a-induced transcriptional activity-in TOPFLASH assay. As shown in FIG. 13, potent inhibitory activity (>80%) was observed for Fabs 5014, 5018-5023, 5025, 5036, 5037 and 6495. Smaller amounts of inhibition (10-35%) was observed for Fabs 5027-5031, 6497-6499; no inhibition was seen for Fabs 5017, 5034 and 5035.

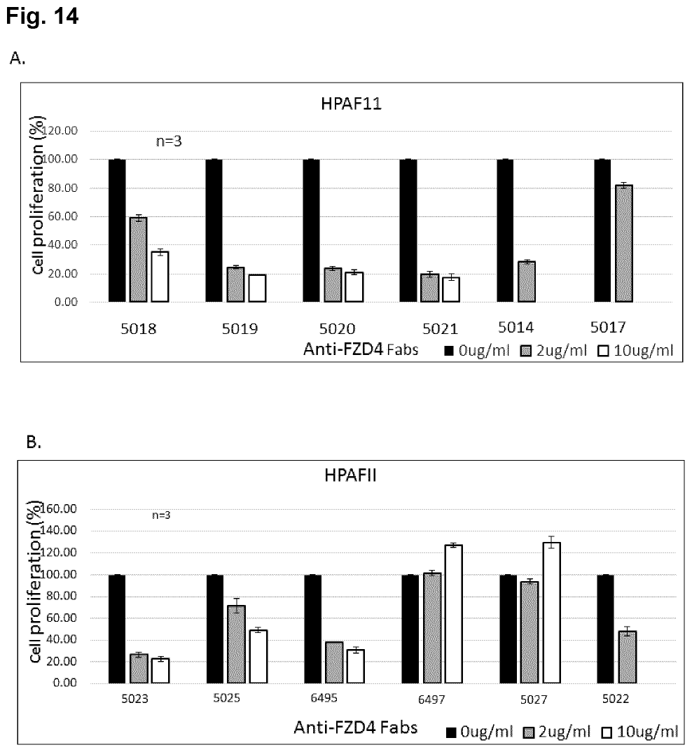

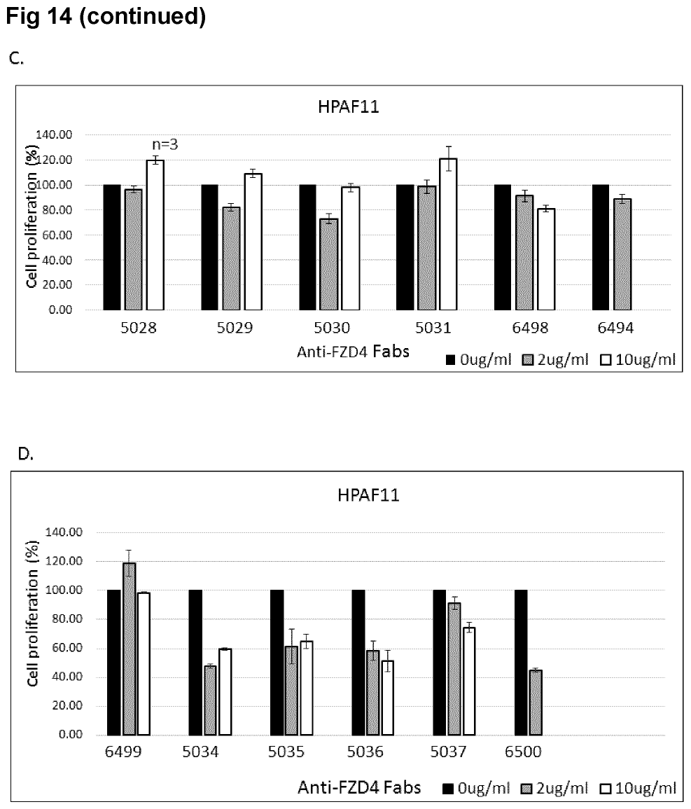

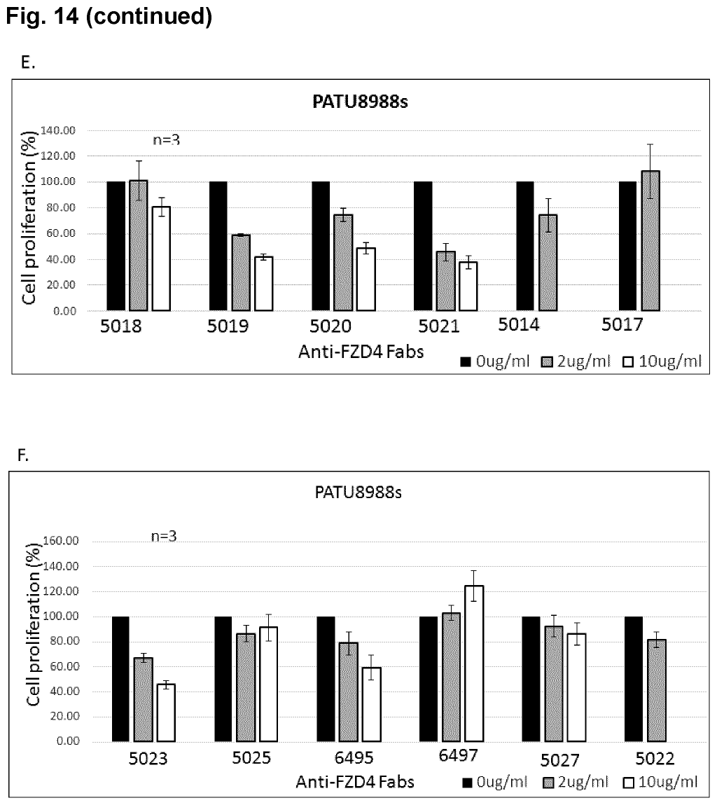

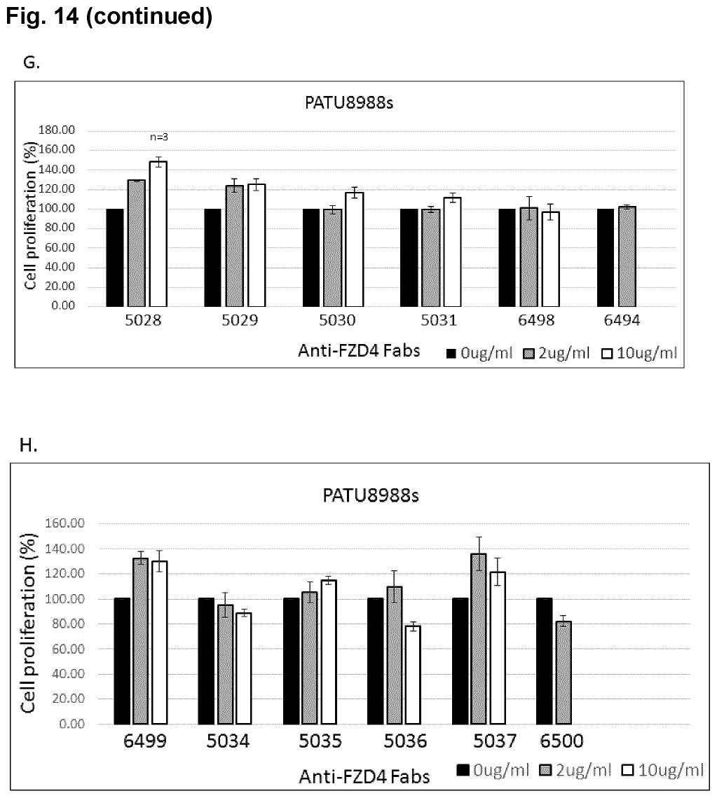

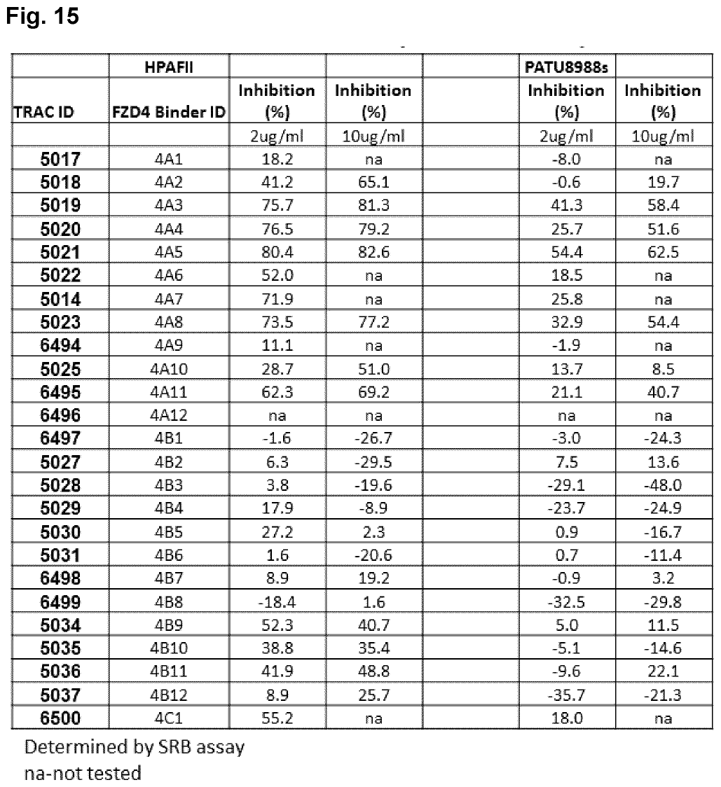

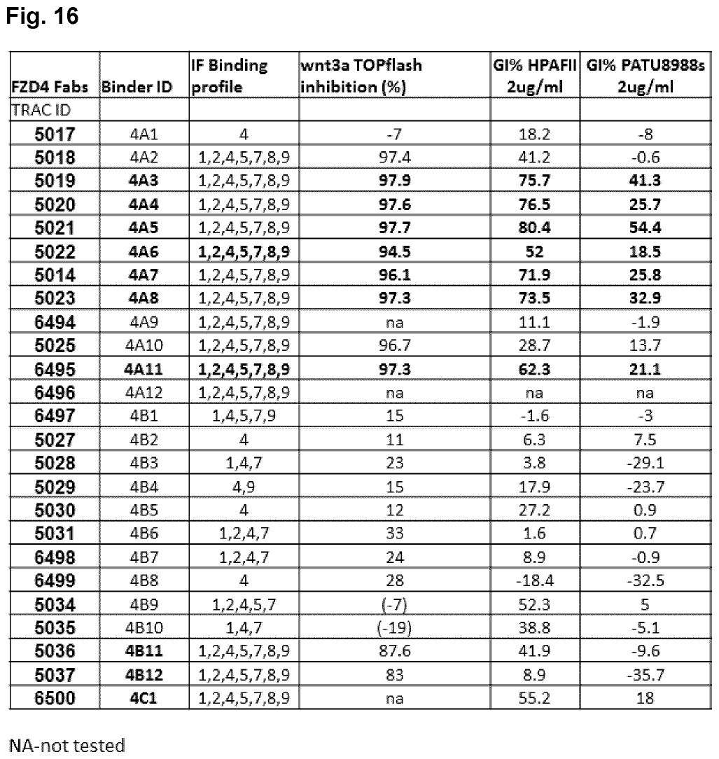

3. Effect on proliferation of cancer cells. To test if the anti-FZD4 Fabs affect proliferation of cancer cells, the pancreatic cancer cells (HPAFII and PATU8988s) were treated with the Fabs at 2 and 10 μg/ml and cell proliferation measured (see FIG. 14A-H). The data was summarized in FIG. 15. The Fabs that were anti-proliferative in a dose-dependent manner include: Fabs 5018-5021, 5023, 5036 and 6495. Fabs 5022 and 6500 were only tested at a single dose level (2 μg/ml) and inhibitory to both cell lines tested. Several Fabs (5017, 5025, 5035 and 5037) were shown to be inhibitory to HPAFII cell line in the assay. Based on the data summarized in FIG. 16, the anti-proliferative activities of the anti-FZD4 Fabs appears to be associated with the following observations: their binding to FZDs 1, 2,4,5,7,8 and 9, and their ability to inhibit Wnt3a-induced transcription activity. The most potent anti-proliferative Fabs include 5014, 5019-5023 and 6495 (FIG. 16).

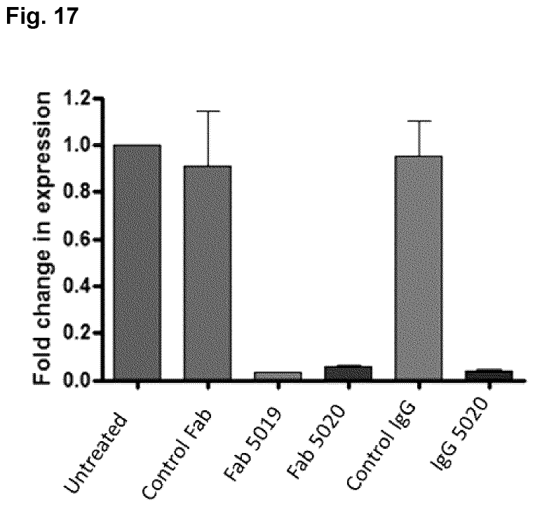

4. Effect on expression of Wnt-regulated gene, Axin2. To characterize the anti-FZD4 antibodies further, several Fabs were converted into IgGs. IgG 5020 and Fabs 5019 and 5020 were tested in the gene expression assay where the mRNA levels of Axin2 gene were measured by RT qPCR. As shown in FIG. 17, IgG5020, Fabs 5019 and 5020 all reduced Axin2 mRNA level in HPAFII cells after the antibody treatment, suggesting these antibodies inhibit the Wnt pathway.

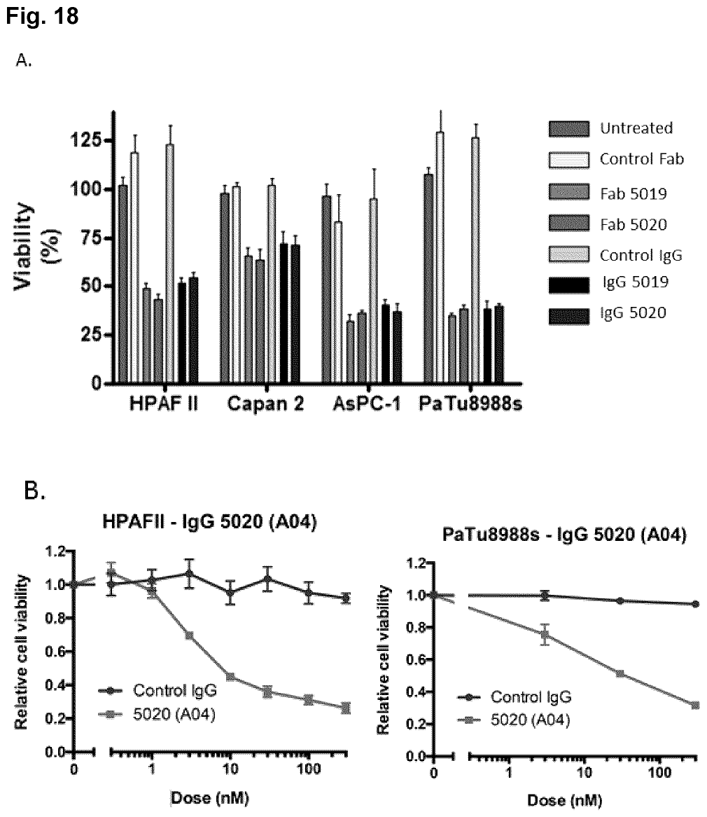

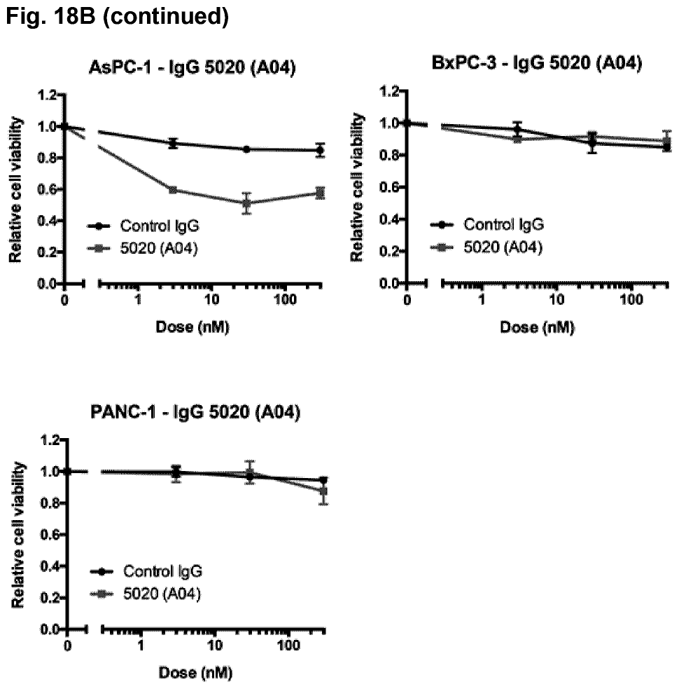

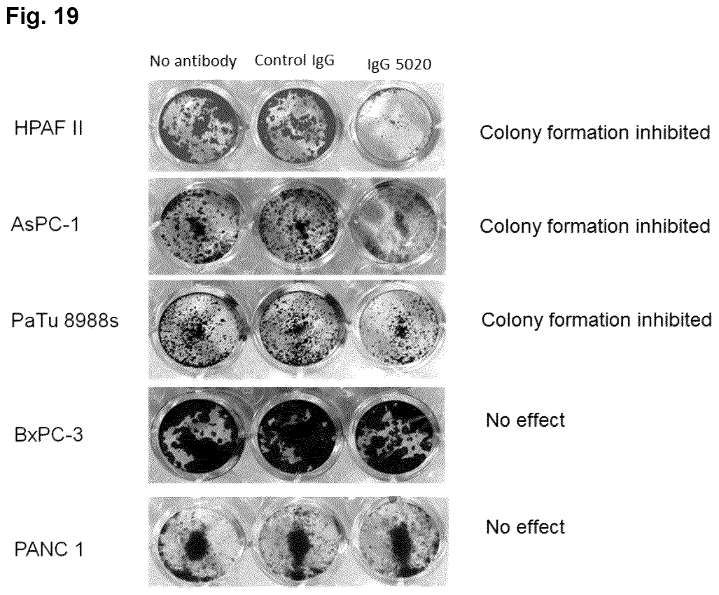

5. Effect of anti-FZD4 IgGs on proliferation of cancer cells. The IgGs were also tested for their effect on proliferation of four pancreatic cancer cell lines HPAFII, CAPAN2, AsPC11 and PATU8988s. These cell lines are all known to harbor damaging mutations in RNF43 gene. As shown in FIG. 18A using alamar blue assays, the proliferation of these pancreatic cells was inhibited by IgGs 5019 and 5020 along with their corresponding Fabs. Furthermore, the IgG5020 was shown to inhibit proliferation of cancer cells in a dose dependent manner (FIG. 18B). Interestingly, several pancreatic cancer cell lines (BxPC3 and PANC 1) that do not have damaging mutations in RNF43 gene were also tested but were not sensitive to IgG5020, suggesting the sensitivity to the FZD4 antibody IgG 5020 may depend on mutations in RNF43 gene. RNF43 and ZNRF3 are Wnt target genes coding for transmembrane E3 ubiquitin ligases targeting Frizzled receptors, whose loss-of-function mutations lead to high expression of FZDs and may sensitize tumor cells to the inhibition of Wnt-dependent signaling. To see if the FZD4 antibodies affect also the colony formation, IgG5020 was tested on 5 pancreatic cancer cell lines (FIG. 19). Consistent with the results in FIG. 18, IgG5020 inhibits colony formation by the RNF43 mutation-harboring cell lines (HPAFII, AsPC1 and PATU8988s) but not that of the non-RNF43 mutation harboring cell lines (BxPC3 and PANC 1).

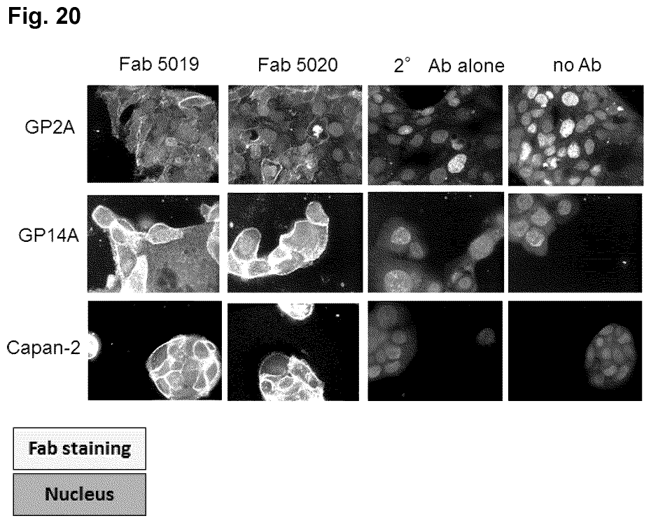

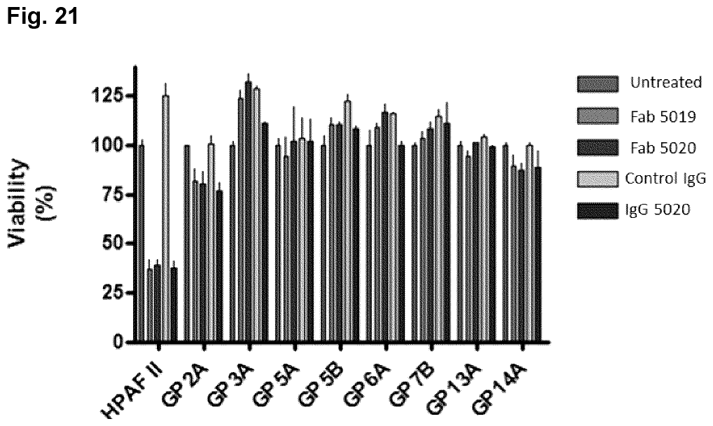

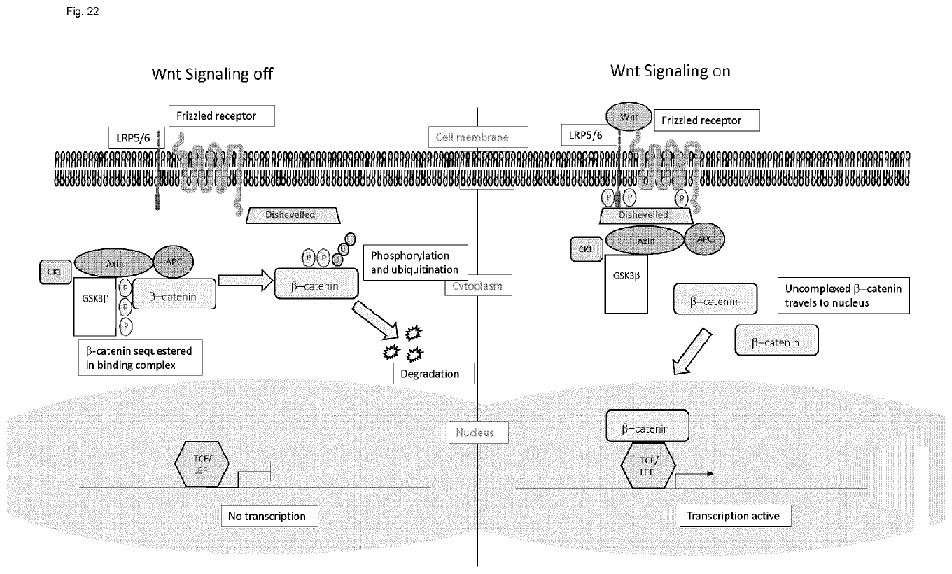

6. Anti-FZD4 Fabs bind to pancreatic cancer patient tumor derived cells effect on their proliferation. Next, Fabs 5019 and 5020 were tested for their binding to pancreatic cancer patient tumor derived cells (PDX) by immunofluorescent staining. As shown in FIG. 20, membrane staining patterns were clearly shown for both Fabs on PDX cell lines GP2A and GP14A as well as on pancreatic cell line CAPAN 2. Furthermore, these antibodies were tested in a cell proliferation assay (FIG. 21). Both Fabs and IgG 5020 were able to inhibit proliferation of the PDX cell line GP2A and GP14A, both of these PDX cell lines that harbor mutations in RNF43 gene, in line with the observation in FIGS. 18 and 19.

In vivo efficacy studies are in progress to demonstrate the anti-tumor activity of these disclosed anti-FZD antibodies.

### XI. Exemplary Embodiments

1. An antibody that specifically binds a cysteine rich domain (CRD) of each of one or a plurality of human Frizzled receptors selected from FZD 1, 2, 4, 5, 7, 8 and 9, comprising a light chain variable region and/or a heavy chain variable region, the heavy chain variable region comprising complementarity determining regions CDR-H1, CDR-H2 and CDR-H3, the light chain variable region comprising complementarity determining region CDR-L1, CDR-L2 and CDR-L3, wherein the amino acid sequences of said CDRs comprise or consist of sequences selected from sequences in Table 1a or 3a.

2. The antibody of embodiment 1 wherein the amino acid sequences of said CDRs comprise or consist of sequences selected from the sequences as set forth below:
CDR-H1 is selected from the group consisting of LYYTDM, IYFSSI, IGSSSI, VNSSSI, IHFSSI, IYSASI, IHSSSI, IYFSSI, IYSSSI, LSYSFF, LSFYFL, LSSYYM, SSFYFM, LSYYYM, IASYFT, FSSSSI, LSYYFM, IYYYPM, FSAYNI, IYYFGM, IHSSSI and ISYHYM;
CDR-H2 is selected from the group consisting of SISLFFGYVS, SNYPSFGSNS, SIYSAFASTS, AFYSSFGATS, AYYSAFASSS, CSYPSFGSTS, SRYPSFGSTS, AIYSSFSANS, SNYPAFGSTS, SIYSAFLSTT, SIYPSSGYTY, SIYPYSGYTY, SIYPFHASTY, TVYPYLDYTY, SIYPYSRNTF, SIYPFSGYST, SIYPYYAYTY, SIYLSFGYGY, CCNSAYRYGP, SIYPYAGNTY, SFYSYYSFTY, SLYTSYGYTY, YIYPFNGYSY, YIYPSYDYTY, YISPPYGFTY, ATYSSFGSIT and SIYPNLGYTY;
CDR-H3 is selected from the group consisting of YHHPFGYAL, YLAM, YHFPFAYSL, YHFPFGFAL, YHFPFGHAL, YHYPFGHAL, YHYPFGHAL, YHYPFGTAL, YHYPFGYAL, YHYPFGYAM, YHYPHGHAL, PAPFSYHVL, AAPGSYHPM, AAPYFYGVM, AFPGSYHPM, AYPFSYHFM, PSAFSYHPM, PVAGAYHPM, SSLGFYNGM, TVRGSKKPYFSGWAM, TYPGYYYIL, SGVGGDHAL, VWYWQ, GYFYTWGGM, GYFYTWGGM, GYYYSWGGM, YHHPFGYAL and AYPFSYHYM
CDR-L1 is SVSSA;
CDR-L2 is SASSLYS; and/or
CDR-L3 is selected from the group consisting of AAYHWPPLF, GVYLF, SSYSLI, WAYGPF, YYHPI and YYSLF.

3. The antibody of embodiment 1 wherein the amino acid sequences of said CDRs comprise or consist of sequences selected as set forth below:
CDR-H1 is selected from the group consisting of ISYYYM, IYSYYM, LSYYYM, IYYYSI, LYSYYM, LSSYSM, ISYYYI, LSYSSM, IYYYYM, LYYYSI, ISSYYI, FSSSSI, LSYYSI, LYSYYI, LSSYYM, LSYYYI, ISSYYM, LSYYSM, LYSYSI, LYYYYI, IYSYYI, ISYSYI and ISYYSM;
CDR-H2 is selected from the group consisting of SIYSYYGYTY, SIYSSSSSTY, SIYPSSSYTY, SIYSSSSYTS, YISSYSGSTY, SIYSSYGYTY, YISSYYGYTY, SIYPSSSSTY, SIYSSSGYTY, YISSYSGSTS, SIYSYYGSTY, SIYSYYGSTY, SIYPYSGYTY, YISPYYGYTS, SISSSSGYTY, SIYSYSSSTY, SISPSSSYTY, YISPYYGYTY, SISPYSSSTY, SIYSSYGSTY, SIYSSSSYTY, SIYPSSGYTY, SIYPYSGSTY, SIYPSYGSTY, YISSYSSYTY, SIYSYYSSTY, YISSSYGYTS, SISPYSSYTY, YISPYSGYTS, SIYPYYSYTY, SISPYYGYTS, SISPSYSSTY, SISSSYSSTY, SIYPYSGSTS, SISSYYSSTS and SIYSYSGYTY;
CDR-H3 is selected from the group consisting of SSFSWAM, SSFYWAL, SWFGWGI, YWFSYGYASYPAF, HPWYGM, SAFYWAL, PAPGHWGF, SSFFWAM, SAFYWAM, HFFAM, SWWAWAF, SAFGWAL, SSFFFAM, PYYWSGGF, HPSSSWFSFGAL, SAFYWAF, SSYAWAM, SSFYWAI, SPWGSGWAGF, PAVWVGL, SWVFWAL, SWVYWGM, SWVYWAL, SSYAWAI, SSFYWAM, HGASFGSGAPAF, SCFFWAM, WAFFGL, SSFYFAM, SAFSWAI, SGFYWAL, PSVGYAAF, SWVGWGL, SSVGYVAM, SWVYWAF, YYYSSSVYFWYAAL, SSFFWAI, SWVYWAI, SWVGWGI, SSVYWAL, WGGWGSGGYFYAAL, FWYPGM and SSFAWAF;
CDR-L1 is SVSSA;
CDR-L2 is SASSLYS; and/or
CDR-L3 is selected from the group consisting of HPWSGGYLI, PVGYWGVPI, VSGGAHALI, VSSAYPI, FWGVPI, SYYHYAALI, WYYAPI, SHSYSLI, SGYGPF, SWSSPI, HYSVYASLI, PHPPSLI, VAYSHVGLI, GYGAPI, SWYSLI, PGYLF, VWFGLI, VYYGSPLF, HAHSPLI, SSAYYPF, GHASPI, SSGGWSLI, VAWSSFLI, SVAAASLI, SGWWGVSLI, SYAAYLF, HGSLF, YAGVSNLF, GWPYSALF, SGYYPSLF, SYHSGSGLI, HGYSASLI, APGWALF, GHSSPI, GWPSLF, VPGYPVPI, HYYSHLI, GPASSLI, SVGSSYYLI, YYGPWVLI, AASWGYPF, HWSYPI and GGWGPF.

4. The antibody of embodiment 2 or 3, wherein the antibody comprises a heavy chain variable region comprising:
i) a heavy chain amino acid sequence as set forth in Table 2;
ii) an amino acid sequence with at least 50%, at least 60%, at least 70%, at least 80%, or at least 90%, at least 95%, at least 98%, at least 99% sequence identity to the heavy chain amino acid sequence as set forth in Table 2, wherein the CDR sequences are a CDR sequence set as set forth in Table 1a or 3a, or
iii) a conservatively substituted amino acid sequence of i) wherein the CDR sequences are a CDR sequence set as set forth in Table 1a or 3a.

5. The antibody of any one of embodiments 2 to 4, wherein the antibody comprises a light chain variable region comprising:
i) a light chain amino acid sequence as set forth in Table 2;
ii) an amino acid sequence with at least 50%, at least 60%, at least 70%, at least 80%, at least 90%, at least 95%, at least 98%, at least 99% sequence identity to the light chain amino acid sequence as set forth in Table 2, wherein the CDR sequences are a CDR sequence set as set forth in Table 1a or 3a, or
iii) a conservatively substituted amino acid sequence of i) wherein the CDR sequences are a CDR sequence set as set forth in Table 1a or 3a.

6. The antibody of any of embodiments 1 to 5, wherein the CDR sequences are a full CDR sequence set selected from an antibody identified in Table 1a or 3a.

7. The antibody of any of embodiments 1 to 5, wherein the CDR sequences comprise a light chain or a heavy chain CDR sequence set selected from an antibody identified in Table 1a or 3a.

8. The antibody of any of embodiments 1 to 7, wherein antibody specifically binds FZD4.

9. The antibody of embodiment 8, wherein the CDR sequences are a CDR sequence set of an antibody selected from antibodies 5017, 5027, 5030, 6499, 5038, 5040-5044, 5046, 5047, 5049-5053, 5055, 5058-5064, 5066, 5068-5072, 5077-5080 or 5081.

10. The antibody of any one of embodiments 1 to 7, wherein the antibody specifically binds FZD4 and at least one other FZD receptor selected from FZD1, FZD2, FZDS, FZD7, FZD8 and FZD9.

11. The antibody of embodiment 9, wherein the CDR sequences are a CDR sequence set of an antibody selected from antibodies 5014, 5016, 5018-5023, 5025, 5028, 5029, 5031, 5034, 5035, 5036, 5037, 6494, 6495, 6496, 6497, 6498, 6500, 5039, 5045, 5048, 5054, 5056, 5057, 5067, and 5073-5076.

12. The antibody of any one of embodiments 1 to 11, wherein the antibody preferentially binds Frizzled 4 (FZD4) compared to FZD1, 2, 5, 7, 8 or 9.

13. The antibody of any of embodiments 1 to 11, wherein antibody preferentially binds FZD4 relative to another FZD receptor.

14. The antibody of embodiment 13, wherein the antibody comprises the CDR sequences are a CDR sequence set of an antibody selected from antibodies 5028, 5029, 5031, 5034, 5035, 6497, 6498, 5039, 5045, 5048, 5054, 5056, 5057, 5067, 5073, 5074, 5075.

15. The antibody of any one of embodiments 1 to 13, wherein the antibody has a binding affinity measured by surface plasmon resonance of between about 0.2 nM and about 15.3 nM.

16. The antibody of any one of embodiments 1 to 15 wherein the antibody is a monoclonal antibody.

17. The antibody of any one of embodiments 1 to 16, wherein the antibody is a humanized antibody.

18. The antibody of any one of embodiments 1 to 17, wherein the antibody is a single chain antibody.

19. The antibody of any one of embodiments 1 to 18, wherein the antibody is an antibody binding fragment selected from Fab, Fab′, F(ab′)2, scFv, dsFv, ds-scFv, dimers, nanobodies, minibodies, diabodies, and multimers thereof.

20. The antibody of any one of embodiments 1 to 18, wherein the antibody is a polyvalent antibody that is divalent, trivalent or tetravalent antibody.

21. The antibody of any one of embodiments 1 to 18, wherein the antibody is a bispecific antibody that further binds to LPR ⅚.

22. The antibody of any one of embodiments 1 to 18, comprising a non-natural glycosylation pattern.

23. The antibody of any one of embodiments 1 to 18, comprising a cysteine substitution or addition in the constant region or a framework region.

24. The antibody of any one of embodiments 1 to 18, which blocks binding of Wnt to FZD.

25. An immunoconjugate comprising the antibody of any one of embodiments 1 to 21 and a detectable label or cytotoxic agent.

26. The immunoconjugate of embodiment 25, comprising a cytotoxic agent selected from maytansinoid, auristatin, dolastatin, tubulysin, cryptophycin, pyrrolobenzodiazepine (PBD) dimer, indolinobenzodiazepine dimer, alpha-amanitin, trichothene, SN-38, duocarmycin, CC1065, calicheamincin, an enediyne antibioatic, taxane, doxorubicin derivatives, anthracycline and stereoisomers, azanofide, isosteres, analogs or derivatives thereof.

27. A nucleic acid molecule encoding the antibody of any one of embodiments 1 to 21.

28. The nucleic acid molecule of embodiment 27, wherein one or more of the CDR sequences is/are encoded by a nucleic acid in Table 1b, 1c, 3b or 3c.

29. The nucleic acid molecule of embodiment 27, wherein the antibody comprises a heavy chain variable region encoded by a nucleic acid comprising:
i) a heavy chain nucleic acid sequence as set forth in Table 2;
ii) a nucleotide sequence with at least 50%, at least 60%, at least 70%, at least 80%, or at least 90% sequence identity to the heavy chain nucleic acid sequence as set forth in Table 2, wherein the CDR sequences are a CDR sequence set as set forth in Table 1a or 3a, or
iii) a codon-degenerate nucleic acid sequence of i) wherein the CDR sequences are a CDR sequence set as set forth in Table 1a or 3a.

30. The nucleic acid molecule of embodiment 27, wherein the antibody comprises a light chain variable region encoded by a nucleic acid comprising:
i) a light chain nucleic acid sequence as set forth in Table 2,
ii) a nucleic acid sequence with at least 50%, at least 60%, at least 70%, at least 80%, at least 90%, at least 95%, at least 98%, at least 99% sequence identity to the light chain nucleic acid sequence as set forth in Table 2, wherein the CDR sequences are a CDR sequence set as set forth in n Table 1a or 3a, or
iii) a codon-degenerate nucleic acid sequence of i) wherein the CDR sequences are a CDR sequence set as set forth in Table 1a or 3a.

31. A vector comprising an expression control sequence operatively linked to the nucleic acid of any one of embodiments 27 to 30.

32. A host cell comprising recombinant nucleic acid molecule comprising an expression control sequence operatively linked to the nucleic acid of any one of embodiments 27 to 30.

33. The host cell of embodiment 32 that is a Chinese Hamster Ovary (CHO) cell.

34. A host cell comprising the vector of embodiment 31.

35. A method for making an anti-FZD antibody comprising culturing a host cell of any one of embodiments 32 to 34.

36. A composition comprising the antibody of any one or more of embodiments 1 to 24, the immunoconjugate of embodiments 25-26, the nucleic acid molecule of embodiments 27-30, the vector of embodiment 31 or the host cell of embodiment 34-34, optionally with a suitable diluent.

37. The composition of embodiment 3636, wherein the composition comprises one or more antibodies or immunoconjugates, optionally wherein the composition is a pharmaceutical composition.

38. A kit comprising the antibody of any one or more of embodiments 1 to 24, the immunoconjugate of embodiments 25-26, the nucleic acid molecule of embodiments 27-30, the vector of embodiment 31 or the host cell of embodiments 34-34.

39. A method of detecting FZD expression, the method comprising contacting a sample comprising one or more cells with one or more antibody or immunoconjugate of any one of embodiments 1 to 26 under conditions permissive for forming an antibody:cell complex and detecting the presence of any antibody complex.

40. The method of embodiment 39, wherein the detection is by immunofluorescence.

41. The method of embodiment 39, wherein the detection is by flow cytometry.

42. The method of any one of embodiments 39 to 41, wherein the method is for detecting FZD4 expression and the antibody or immunoconjugate comprises a CDR sequence set corresponding to an antibody selected from 5017, 5027, 5030, 6499, 5038, 5040-5044, 5046, 5047, 5049-5053, 5055, 5058-5064, 5066, 5068-5072, and 5077-5081.

43. A method of inhibiting a Wnt ligand binding to a FZD receptor, disrupting a Wnt signalling pathway, inhibiting Wnt-induced transcriptional activity, inhibiting activation of disheveled, promoting preservation of the beta-catenin destruction complex of the beta-catenin destruction complex, promoting accumulation of beta-catenin or inhibiting growth of a cell, the method comprising contacting a cell expressing a FZD receptor with an antibody or immunoconjugate of any one of embodiments 1 to 26.

44. The method of embodiment 43, wherein the Wnt ligand is Wnt3a.

45. The method of embodiment 43, wherein the antibody or immunoconjugate comprises a CDR sequence set corresponding to an antibody selected from a) 5017, 5027, 5030, 6499, 5038, 5040-5044, 5046, 5047, 5049-5053, 5055, 5058-5064, 5066, 5068-5072, and 5077-5081 or b) 5014, 5016, 5018-5023, 5025, 5028, 5029, 5031, 5034, 5035, 5036, 5037, 6494, 6495, 6496, 6497, 6498, 6500, 5039, 5045, 5048, 5054, 5056, 5057, 5067, and 5073-5076.

46. A method of treating cancer in a subject in need thereof comprising administering to the subject an effective amount of a pharmaceutical composition comprising an antibody or immunoconjugate of any one of embodiments 1 to 26.

47. The method of embodiment 46, wherein the cancer is selected from colon, lung, breast ovarian, endometrial, pancreas, stomach, liver, adrenocortical carcinoma and osteoblastoma cancer cells.

48. The method of embodiment 46, wherein the cancer is selected from acute myeloid leukemia, neuroblastoma, liver cancer, lung cancer, endometrial cancer, salivary adenoid cystic carcinoma cancer, colorectal cancer, prostate cancer, glioblastoma, bladder cancer cervical cancer, pancreatic cancer, colon cancer, breast cancer, esophageal cancer, glioma, gastric cancer, astrocytoma, and osteosarcoma.

49. The method of embodiment 46, wherein the antibody or immunoconjugate that specifically binds FZDs 1, 2, 4, 5, 7, 8 and 9 in at least one assay, and inhibits Wnt3a-induced signalling in at least one assay, optionally wherein the antibody or immunoconjugate is the antibody or immunoconjugate of any one of embodiments 1 to 26.

50. The method of embodiment 46, wherein the antibody or immunoconjugate comprises a CDR sequence set corresponding to an antibody selected from a) 5017, 5027, 5030, 6499, 5038, 5040-5044, 5046, 5047, 5049-5053, 5055, 5058-5064, 5066, 5068-5072, and 5077-5081 or b) 5014, 5016, 5018-5023, 5025, 5028, 5029, 5031, 5034, 5035, 5036, 5037, 6494, 6495, 6496, 6497, 6498, 6500, 5039, 5045, 5048, 5054, 5056, 5057, 5067, and 5073-5076.

51. The method of embodiment 46, wherein the antibody or immunoconjugate comprises a CDR sequence set corresponding to an antibody selected from 5019 and 5020.

52. The method of embodiment 51, wherein the cancer treated by the method comprises one or more cancer cells comprising a mutation in RNF43 gene and the antibody and the antibody or immunoconjugate comprises a CDR sequence set corresponding to antibody 5020.

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The phrase “at least one” includes “one or more”, “one or a plurality” and “a plurality”. The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The term “consisting essentially of” refers to the inclusion of recited elements and other elements that do not materially affect the basic and novel characteristics of a claimed combination.

Terms of degree such as “about”, “substantially”, and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

Further, the definitions and embodiments described in particular sections are intended to be applicable to other embodiments herein described for which they are suitable as would be understood by a person skilled in the art. For example, in the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

It should be understood that the description and the drawings are not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. Headings used herein are for organizational purposes only and are not meant to be used to limit the scope of the description.

All publications, patents, and patent applications mentioned in this specification are herein incorporated by reference to the same extent as if each individual publication, patent, or patent application was specifically and individually indicated to be incorporated by reference.

1. An antibody that specifically binds a cysteine rich domain (CRD) of each of one or a plurality of human Frizzled receptors selected from FZD 1, 2, 4, 5, 7, 8 and 9, comprising a light chain variable region and/or a heavy chain variable region, the heavy chain variable region comprising complementarity determining regions CDR-H1, CDR-H2 and CDR-H3, the light chain variable region comprising complementarity determining region CDR-L1, CDR-L2 and CDR-L3, wherein the amino acid sequences of said CDRs comprise or consist of sequences selected from sequences in Table 1a or 3a.

2. The antibody of claim 1 wherein the amino acid sequences of said CDRs comprise or consist of sequences selected from the sequences as set forth below:
CDR-H1 is selected from the group consisting of LYYTDM, IYFSSI, IGSSSI, VNSSSI, IHFSSI, IYSASI, IHSSSI, IYFSSI, IYSSSI, LSYSFF, LSFYFL, LSSYYM, SSFYFM, LSYYYM, IASYFT, FSSSSI, LSYYFM, IYYYPM, FSAYNI, IYYFGM, IHSSSI and ISYHYM;
CDR-H2 is selected from the group consisting of SISLFFGYVS, SNYPSFGSNS, SIYSAFASTS, AFYSSFGATS, AYYSAFASSS, CSYPSFGSTS, SRYPSFGSTS, AIYSSFSANS, SNYPAFGSTS, SIYSAFLSTT, SIYPSSGYTY, SIYPYSGYTY, SIYPFHASTY, TVYPYLDYTY, SIYPYSRNTF, SIYPFSGYST, SIYPYYAYTY, SIYLSFGYGY, CCNSAYRYGP, SIYPYAGNTY, SFYSYYSFTY, SLYTSYGYTY, YIYPFNGYSY, YIYPSYDYTY, YISPPYGFTY, ATYSSFGSIT and SIYPNLGYTY;
CDR-H3 is selected from the group consisting of YHHPFGYAL, YLAM, YHFPFAYSL, YHFPFGFAL, YHFPFGHAL, YHYPFGHAL, YHYPFGHAL, YHYPFGTAL, YHYPFGYAL, YHYPFGYAM, YHYPHGHAL, PAPFSYHVL, AAPGSYHPM, AAPYFYGVM, AFPGSYHPM, AYPFSYHFM, PSAFSYHPM, PVAGAYHPM, SSLGFYNGM, TVRGSKKPYFSGWAM, TYPGYYYIL, SGVGGDHAL, VWYVVQ, GYFYTWGGM, GYFYTWGGM, GYYYSWGGM, YHHPFGYAL and AYPFSYHYM
CDR-L1 is SVSSA;
CDR-L2 is SASSLYS; and/or
CDR-L3 is selected from the group consisting of AAYHWPPLF, GVYLF, SSYSLI, WAYGPF, YYHPI and YYSLF.

3. The antibody of claim 1 wherein the amino acid sequences of said CDRs comprise or consist of sequences selected as set forth below:
CDR-H1 is selected from the group consisting of ISYYYM, IYSYYM, LSYYYM, IYYYSI, LYSYYM, LSSYSM, ISYYYI, LSYSSM, IYYYYM, LYYYSI, ISSYYI, FSSSSI, LSYYSI, LYSYYI, LSSYYM, LSYYYI, ISSYYM, LSYYSM, LYSYSI, LYYYYI, IYSYYI, ISYSYI and ISYYSM;
CDR-H2 is selected from the group consisting of SIYSYYGYTY, SIYSSSSSTY, SIYPSSSYTY, SIYSSSSYTS, YISSYSGSTY, SIYSSYGYTY, YISSYYGYTY, SIYPSSSSTY, SIYSSSGYTY, YISSYSGSTS, SISSYYGSTY, SIYSYYGSTY, SIYPYSGYTY, YISPYYGYTS, SISSSSGYTY, SIYSYSSSTY, SISPSSSYTY, YISPYYGYTY, SISPYSSSTY, SIYSSYGSTY, SIYSSSSYTY, SIYPSSGYTY, SIYPYSGSTY, SIYPSYGSTY, YISSYSSYTY, SIYSYYSSTY, YISSSYGYTS, SISPYSSYTY, YISPYSGYTS, SIYPYYSYTY, SISPYYGYTS, SISPSYSSTY, SISSSYSSTY, SIYPYSGSTS, SISSYYSSTS and SIYSYSGYTY;
CDR-H3 is selected from the group consisting of SSFSWAM, SSFYWAL, SWFGWGI, YWFSYGYASYPAF, HPWYGM, SAFYWAL, PAPGHWGF, SSFFWAM, SAFYWAM, HFFAM, SWWAWAF, SAFGWAL, SSFFFAM, PYYWSGGF, HPSSSWFSFGAL, SAFYWAF, SSYAWAM, SSFYWAI, SPWGSGWAGF, PAVWVGL, SWVFWAL, SWVYWGM, SWVYWAL, SSYAWAI, SSFYWAM, HGASFGSGAPAF, SCFFWAM, WAFFGL, SSFYFAM, SAFSWAI, SGFYWAL, PSVGYAAF, SWVGWGL, SSVGYVAM, SWVYWAF, YYYSSSVYFWYAAL, SSFFWAI, SWVYWAI, SWVGWGI, SSVYWAL, WGGWGSGGYFYAAL, FWYPGM and SSFAWAF;
CDR-L1 is SVSSA;
CDR-L2 is SASSLYS; and/or
CDR-L3 is selected from the group consisting of HPWSGGYLI, PVGYWGVPI, VSGGAHALI, VSSAYPI, FWGVPI, SYYHYAALI, WYYAPI, SHSYSLI, SGYGPF, SWSSPI, HYSVYASLI, PHPPSLI, VAYSHVGLI, GYGAPI, SWYSLI, PGYLF, VWFGLI, VYYGSPLF, HAHSPLI, SSAYYPF, GHASPI, SSGGWSLI, VAWSSFLI, SVAAASLI, SGWWGVSLI, SYAAYLF, HGSLF, YAGVSNLF, GWPYSALF, SGYYPSLF, SYHSGSGLI, HGYSASLI, APGWALF, GHSSPI, GWPSLF, VPGYPVPI, HYYSHLI, GPASSLI, SVGSSYYLI, YYGPWVLI, AASWGYPF, HWSYPI and GGWGPF.

4. The antibody of claim 2 or 3, wherein the antibody comprises a heavy chain variable region comprising:
i) a heavy chain amino acid sequence as set forth in Table 2;
ii) an amino acid sequence with at least 50%, at least 60%, at least 70%, at least 80%, at least 90%, at least 95%, at least 98%, at least 99% sequence identity to the heavy chain amino acid sequence as set forth in Table 2, wherein the CDR sequences are a CDR sequence set as set forth in Table 1a or 3a, or
iii) a conservatively substituted amino acid sequence of i) wherein the CDR sequences are a CDR sequence set as set forth in Table 1a or 3a.

5. The antibody of any one of claims 2 to 4, wherein the antibody comprises a light chain variable region comprising:
i) a light chain amino acid sequence as set forth in Table 2;
ii) an amino acid sequence with at least 50%, at least 60%, at least 70%, at least 80%, at least 90%, at least 95%, at least 98%, at least 99% sequence identity to the light chain amino acid sequence as set forth in Table 2, wherein the CDR sequences are a CDR sequence set as set forth in Table 1a or 3a, or
iii) a conservatively substituted amino acid sequence of i) wherein the CDR sequences are a CDR sequence set as set forth in Table 1a or 3a.

6. The antibody of any one of claims 1 to 5, wherein the CDR sequences are a full CDR sequence set selected from an antibody identified in Table 1a or 3a.

7. The antibody of any one of claims 1 to 5, wherein the CDR sequences comprise a light chain or a heavy chain CDR sequence set selected from an antibody identified in Table 1a or 3a.

8. The antibody of any one of claims 1 to 7, wherein antibody specifically binds FZD4.

9. The antibody of claim 8, wherein the CDR sequences are a CDR sequence set of an antibody selected from antibodies 5017, 5027, 5030, 6499, 5038, 5040-5044, 5046, 5047, 5049-5053, 5055, 5058-5064, 5066, 5068-5072, 5077-5080 or 5081.

10. The antibody of any one of claims 1 to 7, wherein the antibody specifically binds FZD4 and at least one other FZD receptor selected from FZD1, FZD2, FZD5, FZD7, FZD8 and FZD9.

11. The antibody of claim 9, wherein the CDR sequences are a CDR sequence set of an antibody selected from antibodies 5014, 5016, 5018-5023, 5025, 5028, 5029, 5031, 5034, 5035, 5036, 5037, 6494, 6495, 6496, 6497, 6498, 6500, 5039, 5045, 5048, 5054, 5056, 5057, 5067, and 5073-5076.

12. The antibody of any one of claims 1 to 11, wherein the antibody preferentially binds Frizzled 4 (FZD4) compared to FZD1, 2, 5, 7, 8 or 9.

13. The antibody of any one of claims 1 to 11, wherein antibody preferentially binds FZD4 relative to another FZD receptor.

14. The antibody of claim 13, wherein the antibody comprises the CDR sequences are a CDR sequence set of an antibody selected from antibodies 5028, 5029, 5031, 5034, 5035, 6497, 6498, 5039, 5045, 5048, 5054, 5056, 5057, 5067, 5073, 5074, 5075.

15. The antibody of any one of claims 1 to 13, wherein the antibody has a binding affinity measured by surface plasmon resonance of between about 0.2 nM and about 15.3 nM.

16. The antibody of any one of claims 1 to 15 wherein the antibody is a monoclonal antibody.

17. The antibody of any one of claims 1 to 16, wherein the antibody is a humanized antibody.

18. The antibody of any one of claims 1 to 17, wherein the antibody is a single chain antibody.

19. The antibody of any one of claims 1 to 18, wherein the antibody is an antibody binding fragment selected from Fab, Fab′, F(ab′)2, scFv, dsFv, ds-scFv, dimers, nanobodies, minibodies, diabodies, and multimers thereof.

20. The antibody of any one of claims 1 to 18, wherein the antibody is a polyvalent antibody that is divalent, trivalent or tetravalent antibody.

21. The antibody of any one of claims 1 to 18, wherein the antibody is a bispecific antibody that further binds to LPR ⅚.

22. The antibody of any one of claims 1 to 18, comprising a non-natural glycosylation pattern.

23. The antibody of any one of claims 1 to 18, comprising a cysteine substitution or addition in the constant region or a framework region.

24. The antibody of any one of claims 1 to 18, which blocks binding of Wnt to FZD.

25. An immunoconjugate comprising the antibody of any one of claims 1 to 21 and a detectable label or cytotoxic agent.

26. The immunoconjugate of claim 25, comprising a cytotoxic agent selected from maytansinoid, auristatin, dolastatin, tubulysin, cryptophycin, pyrrolobenzodiazepine (PBD) dimer, indolinobenzodiazepine dimer, alpha-amanitin, trichothene, SN-38, duocarmycin, CC1065, calicheamincin, an enediyne antibioatic, taxane, doxorubicin derivatives, anthracycline and stereoisomers, azanofide, isosteres, analogs or derivatives thereof.

27. A nucleic acid molecule encoding the antibody of any one of claims 1 to 21.

28. The nucleic acid molecule of claim 27, wherein one or more of the CDR sequences is/are encoded by a nucleic acid in Table 1b, 1c, 3b or 3c.

29. The nucleic acid molecule of claim 27, wherein the antibody comprises a heavy chain variable region encoded by a nucleic acid comprising:
i) a heavy chain nucleic acid sequence as set forth in Table 2;
ii) a nucleotide sequence with at least 50%, at least 60%, at least 70%, at least 80%, or at least 90% sequence identity to the heavy chain nucleic acid sequence as set forth in Table 2, wherein the CDR sequences are a CDR sequence set as set forth in Table 1a or 3a, or
iii) a codon-degenerate nucleic acid sequence of i) wherein the CDR sequences are a CDR sequence set as set forth in Table 1a or 3a.

30. The nucleic acid molecule of claim 27, wherein the antibody comprises a light chain variable region encoded by a nucleic acid comprising:
i) a light chain nucleic acid sequence as set forth in Table 2,
ii) a nucleic acid sequence with at least 50%, at least 60%, at least 70%, at least 80%, at least 90%, at least 95%, at least 98%, at least 99% sequence identity to the light chain nucleic acid sequence as set forth in Table 2, wherein the CDR sequences are a CDR sequence set as set forth in n Table 1a or 3a, or
iii) a codon-degenerate nucleic acid sequence of i) wherein the CDR sequences are a CDR sequence set as set forth in Table 1a or 3a.

31. A vector comprising an expression control sequence operatively linked to the nucleic acid of any one of claims 27 to 30.

32. A host cell comprising recombinant nucleic acid molecule comprising an expression control sequence operatively linked to the nucleic acid of any one of claims 27 to 30.

33. The host cell of claim 32 that is a Chinese Hamster Ovary (CHO) cell.

34. A host cell comprising the vector of claim 31.

35. A method for making an anti-FZD antibody comprising culturing a host cell of any one of claims 32 to 34.

36. A composition comprising the antibody of any one or more of claims 1 to 24, the immunoconjugate of claims 25-26, the nucleic acid molecule of claims 27-30, the vector of claim 31 or the host cell of claim 34-34, optionally with a suitable diluent.

37. The composition of claim 36, wherein the composition comprises one or more antibodies or immunoconjugates, optionally wherein the composition is a pharmaceutical composition.

38. A kit comprising the antibody of any one or more of claims 1 to 24, the immunoconjugate of claims 25-26, the nucleic acid molecule of claims 27-30, the vector of claim 31 or the host cell of claims 34-34.

39. A method of detecting FZD expression, the method comprising contacting a sample comprising one or more cells with one or more antibody or immunoconjugate of any one of claims 1 to 26 under conditions permissive for forming an antibody:cell complex and detecting the presence of any antibody complex.

40. The method of claim 39, wherein the detection is by immunofluorescence.

41. The method of claim 39, wherein the detection is by flow cytometry.

42. The method of any one of claims 39 to 41, wherein the method is for detecting FZD4 expression and the antibody or immunoconjugate comprises a CDR sequence set corresponding to an antibody selected from 5017, 5027, 5030, 6499, 5038, 5040-5044, 5046, 5047, 5049-5053, 5055, 5058-5064, 5066, 5068-5072, and 5077-5081.

43. A method of inhibiting a Wnt ligand binding to a FZD receptor, disrupting a Wnt signalling pathway, inhibiting Wnt-induced transcriptional activity, inhibiting activation of disheveled, promoting preservation of the beta-catenin destruction complex of the beta-catenin destruction complex, promoting accumulation of beta-catenin or inhibiting growth of a cell, the method comprising contacting a cell expressing a FZD receptor with an antibody or immunoconjugate of any one of claims 1 to 26.

44. The method of claim 43, wherein the Wnt ligand is Wnt3a.

45. The method of claim 43, wherein the antibody or immunoconjugate comprises a CDR sequence set corresponding to an antibody selected from a) 5017, 5027, 5030, 6499, 5038, 5040-5044, 5046, 5047, 5049-5053, 5055, 5058-5064, 5066, 5068-5072, and 5077-5081 or b) 5014, 5016, 5018-5023, 5025, 5028, 5029, 5031, 5034, 5035, 5036, 5037, 6494, 6495, 6496, 6497, 6498, 6500, 5039, 5045, 5048, 5054, 5056, 5057, 5067, and 5073-5076.

46. A method of treating cancer in a subject in need thereof comprising administering to the subject an effective amount of a pharmaceutical composition comprising an antibody or immunoconjugate of any one of claims 1 to 26.

47. The method of claim 46, wherein the cancer is selected from colon, lung, breast ovarian, endometrial, pancreas, stomach, liver, adrenocortical carcinoma and osteoblastoma cancer cells.

48. The method of claim 46, wherein the cancer is selected from acute myeloid leukemia, neuroblastoma, liver cancer, lung cancer, endometrial cancer, salivary adenoid cystic carcinoma cancer, colorectal cancer, prostate cancer, glioblastoma, bladder cancer cervical cancer, pancreatic cancer, colon cancer, breast cancer, esophageal cancer, glioma, gastric cancer, astrocytoma, and osteosarcoma.

49. The method of claim 46, wherein the antibody or immunoconjugate that specifically binds FZDs 1, 2, 4, 5, 7, 8 and 9 in at least one assay, and inhibits Wnt3a-induced signalling in at least one assay, optionally wherein the antibody or immunoconjugate is the antibody or immunoconjugate of any one of claims 1 to 26.

50. The method of claim 46, wherein the antibody or immunoconjugate comprises a CDR sequence set corresponding to an antibody selected from a) 5017, 5027, 5030, 6499, 5038, 5040-5044, 5046, 5047, 5049-5053, 5055, 5058-5064, 5066, 5068-5072, and 5077-5081 or b) 5014, 5016, 5018-5023, 5025, 5028, 5029, 5031, 5034, 5035, 5036, 5037, 6494, 6495, 6496, 6497, 6498, 6500, 5039, 5045, 5048, 5054, 5056, 5057, 5067, and 5073-5076.

51. The method of claim 46, wherein the antibody or immunoconjugate comprises a CDR sequence set corresponding to an antibody selected from 5019 and 5020.

52. The method of claim 51, wherein the cancer treated by the method comprises one or more cancer cells comprising a mutation in RNF43 gene and the antibody and the antibody or immunoconjugate comprises a CDR sequence set corresponding to antibody 5020.
